(19) United States
(12) Patent Application Publication
(10) Pub. No.: US 2025/0230163 A1
(43) Pub. Date: Jul. 17, 2025

(54) SOLID FORMS OF HIV INTEGRASE INHIBITORS

(71) Applicant: Gilead Sciences, Inc., Foster City, CA (US)

(72) Inventors: Eric J. Alexy, Belmont, CA (US); Xiaochun Han, San Jose, CA (US); Daniel R. Holycross, Daly City, CA (US); Elizabeth M. Horstman, San Mateo, CA (US); Henry G. Morrison, Dublin, CA (US); Justin Y. Su, Foster City, CA (US); Marisa Thierheimer, Corvallis, OR (US)

(21) Appl. No.: 18/987,492

(22) Filed: Dec. 19, 2024

Related U.S. Application Data
(60) Provisional application No. 63/613,815, filed on Dec. 22, 2023.

(51) Int. Cl. C07D 471/18

(52) U.S. Cl. CPC C07D 471/18

(57) ABSTRACT

The present disclosure relates generally to solid forms of the compound of Formula I:

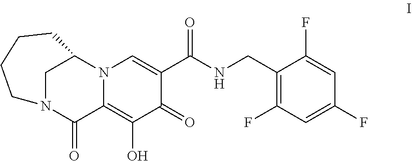 [SMILES: O=C(NCc1c(F)cc(F)cc1F)c1cn2c(c(O)c1=O)C(=O)N1CCCC[C@H]2C1]

Also disclosed are pharmaceutical compositions comprising said solid forms and methods of making said solid forms. The solid forms of the disclosure are useful in treating or preventing human immunodeficiency virus (HIV) infection.

### CROSS REFERENCE TO RELATED APPLICATIONS

This application claims the benefit of U.S. Provisional Application No. 63/613,815, filed on Dec. 22, 2023, the entire contents of which is hereby incorporated by reference in its entirety.

### TECHNICAL FIELD

The present disclosure relates to solid forms of the HIV integrase inhibitor (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide, for use in the treatment of HIV infections. The present disclosure also relates to pharmaceutical compositions containing the solid forms disclosed herein, and methods of treating or preventing HIV infections.

### BACKGROUND

There is an ongoing need for antiviral agents and methods for treating human immunodeficiency virus (HIV) infections. There is also a constant need to develop methods for preparation and purification of the antiviral agents, as well as prepare improved pharmaceutical formulations of the same. The solid forms disclosed herein help meet these and other needs.

### SUMMARY

The present disclosure provides crystalline forms of the compound of Formula I:

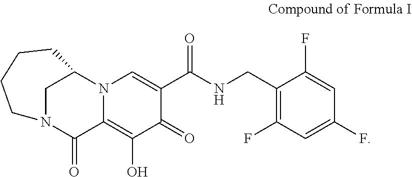 [SMILES: O=C(NCc1c(F)cc(F)cc1F)c1cn2c(c(O)c1=O)C(=O)N1CCCC[C@H]2C1]

The present disclosure also provides salts of the compound of Formula I, as well as crystalline forms of the salts.

The present disclosure also provides co-crystals of the compound of Formula I, as well as crystalline forms of the co-crystals.

The present disclosure further provides a pharmaceutical composition comprising a crystalline form, amorphous form, salt, co-crystal of the compound of Formula I disclosed herein, and a pharmaceutically acceptable excipient.

The present disclosure further provides a kit comprising a crystalline form, amorphous form, salt, co-crystal of the compound of Formula I disclosed herein, and a pharmaceutically acceptable excipient.

A method of treating an HIV infection in a human having or at risk of having the infection, comprising administering to the human a therapeutically effective amount of a crystalline form, amorphous form, salt, or co-crystal disclosed herein.

### BRIEF DESCRIPTION OF THE DRAWINGS

FIG. 1. Shows the XRPD pattern for the compound of Formula I, Form I.

FIG. 2. Shows the DSC curve for the compound of Formula I, Form I.

FIG. 3. Shows the TGA curve for the compound of Formula I, Form I.

FIG. 4. Shows the DVS curve for the compound of Formula I, Form I.

FIG. 5. Shows the atomic displacement ellipsoid drawing of the compound of Formula I, Form I.

FIG. 6. Shows the XRPD pattern for the compound of Formula I, Form II.

FIG. 7. Shows the XRPD pattern for the compound of Formula I, Form III.

FIG. 8. Shows the DSC curve for the compound of Formula I, Form III.

FIG. 9. Shows the TGA curve for the compound of Formula I, Form III.

FIG. 10. Shows the DVS curve for the compound of Formula I, Form III.

FIG. 11. Shows the XRPD pattern for the compound of Formula I, Form IV.

FIG. 12. Shows the DSC curve for the compound of Formula I, Form IV.

FIG. 13. Shows the TGA curve for the compound of Formula I, Form IV.

FIG. 14. Shows the DVS curve for the compound of Formula I, Form IV.

FIG. 15. Shows the XRPD pattern for the compound of Formula I, Amorphous.

FIG. 16. Shows the DSC curve for the compound of Formula I, Amorphous.

FIG. 17. Shows the XRPD pattern for the compound of Formula I, Sodium Salt.

FIG. 18. Shows the DSC curve for the compound of Formula I, Sodium Salt.

FIG. 19. Shows the TGA curve for the compound of Formula I, Sodium Salt.

FIG. 20. Shows the DVS curve for the compound of Formula I, Sodium Salt.

FIG. 21. Shows the XRPD pattern for the compound of Formula I, Potassium Salt, Form I.

FIG. 22. Shows the DSC curve for the compound of Formula I, Potassium Salt, Form I.

FIG. 23. Shows the TGA curve for the compound of Formula I, Potassium Salt, Form I.

FIG. 24. Shows the DVS curve for the compound of Formula I, Potassium Salt, Form I.

FIG. 25. Shows the XRPD pattern for the compound of Formula I, Potassium Salt, Form II.

FIG. 26. Shows the DSC curve for the compound of Formula I, Potassium Salt, Form II.

FIG. 27. Shows the TGA curve for the compound of Formula I, Potassium Salt, Form II.

FIG. 28. Shows the DVS curve for the compound of Formula I, Potassium Salt, Form II.

FIG. 29. Shows the XRPD pattern for the compound of Formula I, Diethylamine Salt.

FIG. 30. Shows the DSC curve for the compound of Formula I, Diethylamine Salt.

FIG. 31. Shows the TGA curve for the compound of Formula I, Diethylamine Salt.

FIG. 32. Shows the DVS curve for the compound of Formula I, Diethylamine Salt.

FIG. 33. Shows the XRPD pattern for the compound of Formula I, Diethanolamine Salt.

FIG. 34. Shows the DSC curve for the compound of Formula I, Diethanolamine Salt.

FIG. 35. Shows the TGA curve for the compound of Formula I, Diethanolamine Salt.

FIG. 36. Shows the DVS curve for the compound of Formula I, Diethanolamine Salt.

FIG. 37. Shows the XRPD pattern for the compound of Formula I, Piperazine Co-Crystal.

FIG. 38. Shows the DSC curve for the compound of Formula I. Piperazine Co-Crystal.

FIG. 39. Shows the TGA curve for the compound of Formula I, Piperazine Co-Crystal.

FIG. 40. Shows the DVS curve for the compound of Formula I, Piperazine Co-Crystal.

FIG. 41. Shows the XRPD pattern for the compound of Formula I, Calcium Salt, Form I.

FIG. 42. Shows the DSC curve for the compound of Formula I, Calcium Salt, Form I.

FIG. 43. Shows the TOA curve for the compound of Formula I, Calcium Salt, Form I.

FIG. 44. Shows the DVS curve for the compound of Formula I, Calcium Salt, Form I.

FIG. 45. Shows the XRPD pattern for the compound of Formula I, Calcium Salt, Form II.

FIG. 46. Shows the DSC curve for the compound of Formula I, Calcium Salt, Form II.

FIG. 47. Shows the TGA curve for the compound of Formula I, Calcium Salt, Form II.

FIG. 48. Shows the XRPD pattern for the compound of Formula I, Calcium Salt, Form III.

FIG. 49. Shows the DSC curve for the compound of Formula I, Calcium Salt, Form III.

FIG. 50. Shows the TGA curve for the compound of Formula I, Calcium Salt, Form III.

FIG. 51. Shows the DVS curve for the compound of Formula I, Calcium Salt, Form III.

FIG. 52. Shows the XRPD pattern for the compound of Formula I, Calcium Salt, Form IV.

FIG. 53. Shows the DSC curve for the compound of Formula I, Calcium Salt, Form IV.

FIG. 54. Shows the TGA curve for the compound of Formula I, Calcium Salt, Form IV.

FIG. 55. Shows the DVS curve for the compound of Formula I, Calcium Salt, Form IV.

FIG. 56. Shows the XRPD pattern for the compound of Formula I, Calcium Salt, Form V.

FIG. 57. Shows the DSC curve for the compound of Formula I, Calcium Salt, Form V.

FIG. 58. Shows the TGA curve for the compound of Formula I, Calcium Salt, Form V.

FIG. 59. Shows the DVS curve for the compound of Formula I, Calcium Salt, Form V.

FIG. 60. Shows the XRPD pattern for the compound of Formula I, Calcium Salt, Form VI.

FIG. 61. Shows the DSC curve for the compound of Formula I, Calcium Salt, Form VI.

FIG. 62. Shows the TGA curve for the compound of Formula I, Calcium Salt, Form VI.

FIG. 63. Shows the DVS curve for the compound of Formula I, Calcium Salt, Form VI.

FIG. 64. Shows the XRPD pattern for the compound of Formula I, Calcium Salt, Amorphous.

FIG. 65. Shows the DSC curve for the compound of Formula I, Calcium Salt, Amorphous.

### DETAILED DESCRIPTION

The present invention relates to new solid forms of the HIV integrase inhibitor (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (Compound of Formula I, see below), which was disclosed in WO2020/197991.

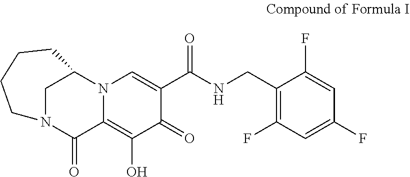 [SMILES: O=C(NCc1c(F)cc(F)cc1F)c1cn2c(c(O)c1=O)C(=O)N1CCCC[C@H]2C1]

The solid forms of the invention include free forms and salt forms (both amorphous and crystalline) as well as cocrystal forms of the compound of Formula I. As used herein, “solid form” generally refers to a solid chemical substance that can be in any morphological form such as, for example, crystalline or amorphous, as well as disordered crystals, liquid crystals, plastic crystals, mesophases, and the like, or any combination thereof. In some embodiments, the solid form of the invention is a salt of the compound of Formula I which can be amorphous or crystalline. In further embodiments, the solid form can be a cocrystal of the compound of Formula I, in which the compound of Formula I has formed a crystalline solid together with a coformer molecule. Both crystalline salts and cocrystals of the compound of Formula I can exist in different crystalline forms (i.e., have different polymorphic or pseudopolymorphic forms).

As used herein, the term “cocrystal” refers to a compound (such as the compound of Formula I) crystallized together with one or more coformer molecules (e.g., molecules other than the compound). Depending on the chemical nature and proportion of coformers present in the cocrystal, different physical properties related to, for example, dissolution and solubility may be observed compared with solid forms of the compound by itself or salts thereof. In some instances, the coformer molecule may be a protic acid, and whether the protic acid forms a salt or a cocrystal will often depend on the relative pKa's of the compound and coformer. See, e.g., Regulatory Classification of Pharmaceutical Co-Crystals: Guidance for Industry, revised August 2016, published by the U.S. Dept. of Health and Human Services, FDA. Center for Drug Evaluation and Research (CDER).

As used herein, “crystalline form” is meant to refer to a certain lattice configuration of a crystalline substance (e.g., a salt or a cocrystal). Different crystalline forms of the same substance typically have different crystalline lattices (e.g., unit cells) which are attributed to different physical properties that are characteristic of each of the crystalline forms. In some instances, different lattice configurations have different water or solvent content giving rise to solvated or hydrated crystalline forms. The term “solvated,” as used herein, is meant to refer to a crystalline form that includes solvent molecules in the crystalline lattice. The term “hydrated,” as used herein, is meant to refer to a crystalline form that is solvated, where the solvent is water and water molecules are included in the crystalline lattice. Example “hydrated” crystalline forms include hemihydrates, monohydrates, dihydrates, and the like. Other hydrated forms such as channel hydrates and the like are also included within the meaning of the term. The term “fully hydrated” is meant to refer to where the water content of the hydrate is present in the expected stoichiometric amounts. The term “partially hydrated” is meant to refer to where the water content of the hydrate is present in less than the expected stoichiometric amounts (e.g., where some of the water of a monohydrate has been removed). Similarly, the term “unsolvated” or “anhydrous” refers to a crystalline form being substantially free of solvent or water, respectively, although some residual solvent or water may be present, for example, left over from the processes used to prepare the crystalline form.

According to the present invention, a crystalline form of a salt or cocrystal of the compound of Formula I can be useful in the synthesis and/or purification of the compound of Formula I. For example, a crystalline form of a salt or cocrystal of the compound of Formula I can be an intermediate in the synthesis of the compound of Formula I. In addition, different crystalline forms of salts and cocrystals of the compound of Formula I may have different properties with respect to bioavailability, stability, purity, and/or manufacturability for medical or pharmaceutical uses. Variations in the crystal structure of a pharmaceutical drug substance or active ingredient may affect the dissolution rate (which may affect bioavailability, etc.), manufacturability (e.g., case of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, the crystalline forms of the salts and cocrystals of the compound of Formula I may provide advantages such as improving the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different crystalline forms of the salts and cocrystals of the compound of Formula I which may exhibit one or more of the favorable characteristics described above. The processes for the preparation of the crystalline and cocrystal forms described herein and characterization of these crystalline and cocrystal forms are described in detail below.

In some embodiments, the pharmaceutically acceptable salts described herein, cocrystals, or crystalline forms thereof, are purified or substantially isolated. By “substantially isolated” is meant that the salt, cocrystal, or crystalline form thereof is at least partially or substantially separated from the environment in which it was formed or detected. Partial separation can include, for example, a composition enriched in the salt, cocrystal, or crystalline form of the invention. Substantial separation can include compositions containing at least about 50%, at least about 60%, at least about 70%, at least about 80%, at least about 90%, at least about 95%, at least about 97%, or at least about 99% by weight of the salt, cocrystal, or crystalline form of the invention. In some embodiments, the salt, cocrystal, or crystalline form of the invention can be prepared with a purity of about 75% or more, 80% or more, 85% or more, 90% or more, 95% or more. 98% or more, or 99% or more.

The different crystalline forms can be identified by solid state characterization methods such as by X-ray powder diffraction (XRPD). Other characterization methods such as differential scanning calorimetry (DSC), thermogravimetric analysis (TGA), and dynamic vapor sorption (DVS) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The present invention provides crystalline forms of certain compounds or salts thereof. In some embodiments, the crystalline form may be substantially anhydrous. In some embodiments, the crystalline form may be hydrated or solvated.

### Compound of Formula I, Form I

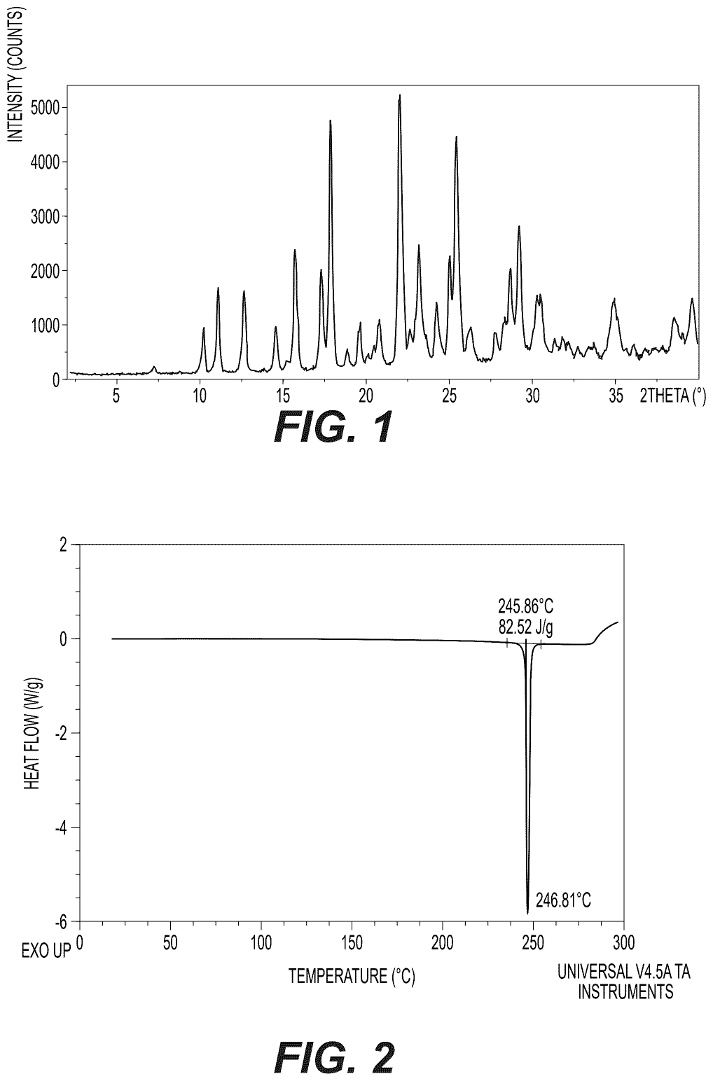

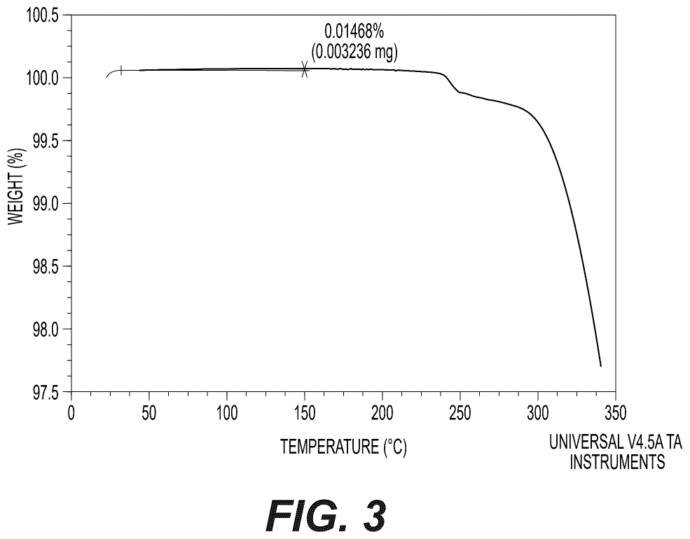

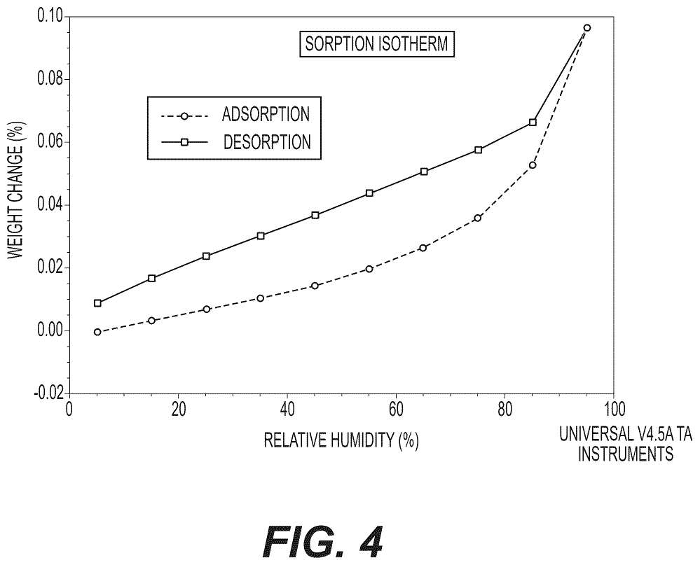

In some embodiments, the present disclosure provides a crystalline Form I of the compound of Formula I (Formula I, Form I), wherein the crystal structure exhibits an X-ray powder diffraction (XRPD) pattern substantially as shown in FIG. 1. Formula I, Form I may exhibit a differential scanning calorimetry (DSC) thermogram substantially as shown in FIG. 2. Formula I, Form I may exhibit a thermogravimetric analysis (TGA) graph substantially as shown in FIG. 3. Formula I, Form I may exhibit a dynamic vapor sorption (DVS) curve substantially as shown in FIG. 4.

In some embodiments of Formula I, Form I, at least one, at least two, at least three, or all of the following (a)-(d) apply: (a) Formula I, Form I has an XRPD pattern substantially as shown in FIG. 1: (b) Formula I, Form I has a DSC thermogram substantially as shown in FIG. 2; (c) Formula I, Form I has a TGA curve substantially as shown in FIG. 3; (d) Formula I, Form I has a DVS curve substantially as shown in FIG. 4.

In some embodiments, Formula I, Form I has the following properties:
(a) an XRPD pattern substantially as shown in FIG. 1;
(b) a DSC thermogram substantially as shown in FIG. 2;
(c) a TOA curve substantially as shown in FIG. 3; and
(d) a DVS curve substantially as shown in FIG. 4.

In some embodiments, Formula I, Form I has an XRPD pattern displaying at least two, at least three, at least four, at least five, or at least six of the degree 2θ-reflections with the greatest intensity as the XRPD pattern substantially as shown in FIG. 1.

In some embodiments, Formula I, Form I has an XRPD pattern comprising degree 20-reflections (+/−0.2 degrees 2θ) at 17.8°, 22.0°, and 25.4°. In some embodiments, Formula I, Form I has an XRPD pattern comprising degree 29-reflections (+/−0.2 degrees 2θ) at 17.8°, 22.0°, and 25.4°, and one, two or three of the degree 2θ-reflections (+/−0.2 degrees 2θ) at 15.6°, 23.1°, and 29.2°. In some embodiments, Formula I, Form I has an XRPD pattern comprising degree 2θ-reflections (+/−0.2 degrees 2θ) at 17.8°, 22.0°, and 25.4°, and one or two of the degree 2θ-reflections (+/−0.2 degrees 2θ) at 15.6°, 23.1°, and 29.2°. In some embodiments, Formula I, Form I has an XRPD pattern comprising degree 2θ-reflections (+/−0.2 degrees 2θ) at 17.8°, 22.0°, and 25.4°, and one of the degree 2θ-reflections (+/−0.2 degrees 2θ) at 15.6°, 23.1°, and 29.2°+0.2°. In some embodiments, Formula I, Form I has an XRPD pattern comprising degree 2θ-reflections (+/−0.2 degrees 2θ) at 17.8°, 22.0°, and 25.4°, and two of the degree 2θ-reflections (+/−0.2 degrees 2θ) at 15.6°, 23.1°, and 29.2°. In some embodiments, Formula I, Form I has an XRPD pattern comprising degree 2θ-reflections (+/−0.2 degrees 2θ) at 15.6°. 17.8°, 22.0°, 23.1°, 25.4°, and 29.2°. In some embodiments, Formula I, Form I has an XRPD pattern comprising any three of the degree 2θ-reflections (+/−0.2 degrees 2θ) selected from the group consisting of 15.6°, 17.8°, 22.0°, 23.1°, 25.4°, and 29.2°.

In some embodiments, Formula I, Form I has an XRPD pattern comprising degree 20-reflections (+/−0.2 degrees 2θ) at 15.6°, 17.8°, 22.0°, 23.19, 25.4°, and 29.2°, and one, two, or three of the degree 2θ-reflections (+/−0.2 degrees 2θ) at 11.12, 12.6°, and 17.3°. In some embodiments, Formula I, Form I has an XRPD pattern comprising degree 2θ-reflections (+/−0.2 degrees 2θ) at 15.6°, 17.8°, 22.0°, 23.1°, 25.4°, and 29.2°, and one or two of the degree 20-reflections (+/−0.2 degrees 2θ) at 11.1°, 12.6°, and 17.3°. In some embodiments, Formula I, Form I has an XRPD pattern comprising degree 2θ-reflections (+/−0.2 degrees 2θ) at 15.6°. 17.8°, 22.0°, 23.1°, 25.4°, and 29.2°, and one of the degree 2θ-reflections (+/−0.2 degrees 2θ) at 11.1°, 12.6°, and 17.3°. In some embodiments, Formula I, Form I has an XRPD pattern comprising degree 26-reflections (+/−0.2 degrees 2θ) at 15.6°, 17.8°, 22.0°, 23.1°, 25.4°, and 29.2°, and two of the degree 2θ-reflections (+/−0.2 degrees 2θ) at 11.19, 12.6°, and 17.3°. In some embodiments, Formula I, Form I has an XRPD pattern comprising degree 2θ-reflections (+/−0.2 degrees 2θ) at 11.1°, 12.6°, 15.6°, 17.3°, 17.8°, 22.0°, 23.1°, 25.4°, and 29.2°. In some embodiments, Formula I, Form I has an XRPD pattern comprising three of the degree 20-reflections (+/−0.2 degrees 2θ) at 11.1°, 12.6°, 15.6°, 17.3°, 17.82, 22.0°, 23.19, 25.4°, and 29.2°. In some embodiments, Formula I, Form I has an XRPD pattern comprising at least three of the degree 29-reflections (+/−0.2 degrees 2θ) at 10.2°, 11.1°, 12.6°, 14.5°, 15.6°, 17.3°, 17.8°, 22.0°, 23.1°, 25.4°, and 29.2°. In some embodiments, Formula I, Form I has an XRPD pattern comprising at least four of the degree 2θ-reflections (+/−0.2 degrees 2θ) at 10.2°, 11.1°, 12.6°, 14.5°, 15.6°, 17.3°, 17.8°, 22.0°, 23.1°, 25.4°, and 29.2°. In some embodiments, Formula I, Form I has an XRPD pattern comprising at least five of the degree 2θ-reflections (+/−0.2 degrees 2θ) at 10.2°, 11.1°, 12.6°, 14.5°, 15.6°, 17.3°, 17.8°, 22.0°, 23.1°, 25.4°, and 29.2°.

In some embodiments, Formula I, Form I has an XRPD pattern comprising peaks at:

| Pos. [°2Th. ± 0.2] | Rel. Int. [%] |
|---|---|
| 10.2051 | 17.91 |
| 11.0588 | 34.41 |
| 12.6118 | 31.59 |
| 14.5193 | 17.32 |
| 15.6463 | 41.62 |
| 17.2528 | 38.69 |
| 17.8322 | 100.00 |
| 19.6384 | 14.92 |
| 20.7623 | 16.29 |
| 21.9574 | 96.32 |
| 23.1489 | 44.24 |
| 24.2050 | 21.41 |
| 25.0185 | 40.07 |
| 25.3612 | 84.53 |
| 26.2356 | 11.77 |
| 27.7314 | 10.03 |
| 28.6533 | 34.12 |
| 29.1593 | 49.06 |
| 30.5358 | 20.39 |
| 34.8604 | 22.44 |
| 38.5013 | 12.61 |

In some embodiments, Formula I, Form I is unsolvated. In some embodiments, Formula I, Form I is anhydrous.

In some embodiments, Formula I, Form I, is characterized by a differential scanning calorimetry (DSC) curve comprising an endothermic transition with an onset at about 246° C.

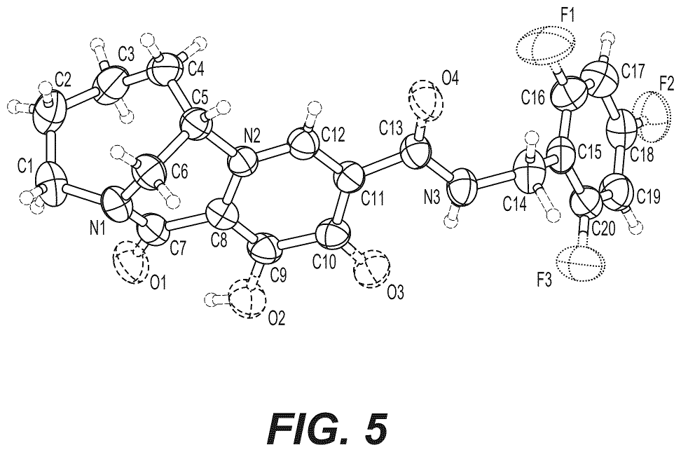

The single crystal data collected on Form I are summarized in Table 1 below and also shown in FIG. 5. The crystal system of Formula I, Form I is monoclinic and the space group is P21. The cell parameters and calculated volume are: a=8.94841 (12) Å, b=8.58582 (16) Å, c=12.59306 (18) Å, a=90°, β=103.2526 (13)°, γ=90°, V=941.75 (3) Å3. The molecular weight is 421.37 g mol⁻¹ with Z=2, resulting in a calculated density of 1.486 g cm⁻³. In some embodiments, Formula I, Form I has space group P2₁ having cell parameters a=8.94 Å, b=8.59 Å, c=12.59 Å, α=90°, β=103.3°, and γ=90°.

TABLE 1
Crystal Data and Data Collection Parameters for Formula I, Form I

| | |
|---|---|
| Empirical formula | C₂₀H₁₈F₃N₃O₄ |
| Formula weight (g mol⁻¹) | 421.37 |
| Temperature (K) | 300.1(2) |
| Wavelength (Å) | 1.54184 |
| Crystal system | monoclinic |
| Space group | P2₁ |
| Unit cell parameters | |
| a = 8.94841(12) Å | α = 90° |
| b = 8.58582(16) Å | β = 103.2526(13)° |
| c = 12.59306(18) Å | γ = 90° |
| Unit cell volume (Å³) | 941.75(3) |
| Cell formula units, Z | 2 |
| Calculated density (g cm⁻³) | 1.486 |
| Absorption coefficient (mm⁻¹) | 1.070 |
| F(000) | 436 |
| Crystal size (mm³) | 0.21 × 0.08 × 0.02 |
| Reflections used for cell measurement | 6168 |
| θ range for cell measurement | 5.0740°-77.2490° |
| Total reflections collected | 9769 |
| Index ranges | −10 ≤ h ≤ 11; −10 ≤ k ≤ 9; −15 ≤ l ≤ 15 |
| θ range for data collection | θₘᵢₙ = 5.078°, θₘₐₓ = 77.513° |
| Completeness to θₘₐₓ | 97.8% |
| Completeness to θ_{full} = 67.684° | 99.8% |
| Absorption correction | multi-scan |
| Transmission coefficient range | 0.891-1.000 |
| Refinement method | full matrix least-squares on F² |
| Independent reflections | 3339 [Rᵢₙₜ = 0.0233, R_{σ} = 0.0230] |
| Reflections [I > 2σ(I)] | 3062 |
| Reflections / restraints / parameters | 3339 / 1 / 343 |
| Goodness-of-fit on F² | S = 1.05 |
| Final residuals [I > 2σ(I)] | R = 0.0303, R_{w} = 0.0811 |
| Final residuals [all reflections] | R = 0.0331, R_{w} = 0.0835 |
| Largest diff. peak and hole (e Å⁻³) | 0.104, −0.149 |
| Max/mean shift/standard uncertainty | 0.000 / 0.000 |
| Absolute structure determination | Flack parameter: 0.00(8) |
| | Hooft parameter: 0.03(7) |
| | Friedel coverage: 66.4% |

### Compound of Formula I, Form II

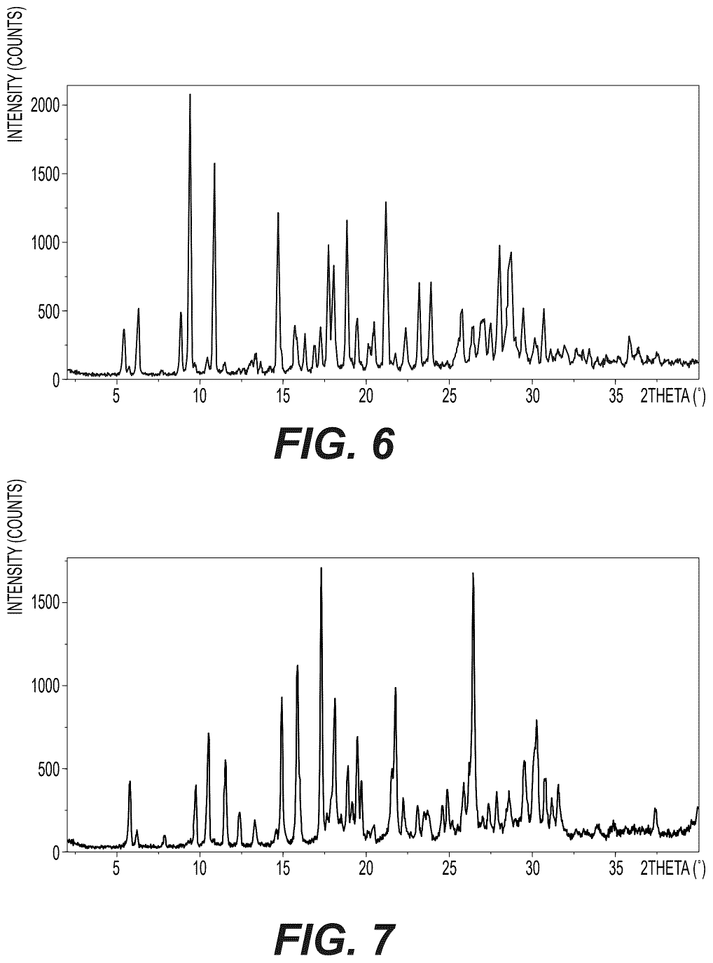

In some embodiments, provided is crystalline Form II of the compound of Formula I (Formula I, Form II), wherein the crystal structure exhibits an X-ray powder diffraction (XRPD) pattern substantially as shown in Figure. 6.

In some embodiments, Formula I, Form II has an XRPD pattern displaying at least two, at least three, at least four, at least five, or at least six of the degree 2θ-reflections with the greatest intensity as the XRPD pattern substantially as shown in Figure. 6.

In some embodiments, Formula I, Form II has an XRPD pattern comprising degree 20-reflections (+/−0.2 degrees 2θ) at 9.5°, 10.9°, and 21.2°. In some embodiments, crystalline Formula I, Form II has an XRPD pattern comprising degree 2θ-reflections (+/−0.2 degrees 2θ) at 9.5°, 10.9°, and 21.2°, and one, two or three of the degree 2θ-reflections (+/−0.2 degrees 2θ) at 14.7°, 17.8°, and 18.9°. In some embodiments, crystalline Formula I, Form II has an XRPD pattern comprising degree 2θ-reflections (+/−0.2 degrees 2θ) at 9.5°, 10.9°, and 21.2°, and one or two of the degree 2θ-reflections (+/−0.2 degrees 2θ) at 14.7°, 17.8°, and 18.9°. In some embodiments, crystalline Formula I, Form II has an XRPD pattern comprising degree 20-reflections (+/−0.2 degrees 2θ) at 9.5°, 10.9°, and 21.2°, and one of the degree 2θ-reflections (+/−0.2 degrees 2θ) at 14.7°, 17.8°, and 18.9°. In some embodiments, crystalline Formula I, Form II has an XRPD pattern comprising degree 2θ-reflections (+/−0.2 degrees 2θ) at 9.5°. 10.9°, and 21.2°, and two of the degree 2θ-reflections (+/−0.2 degrees 2θ) at 14.7°, 17.8°, and 18.9°. In some embodiments, crystalline Formula I, Form II has an XRPD pattern comprising degree 2θ-reflections (+/−0.2 degrees 2θ) at 9.5°, 10.9°, 14.7°, 17.8°, 18.9°, and 21.2°. In some embodiments, crystalline Formula I, Form II has an XRPD pattern comprising any three of the degree 2θ-reflections (+/−0.2 degrees 2θ) selected from the group consisting of 9.5°, 10.9°, 14.7°, 17.8°, 18.9°, and 21.2°.

In some embodiments, crystalline Formula I, Form II has an XRPD pattern comprising 2θ-reflections (+/−0.2 degrees 2θ) at 9.5°, 10.9°, 14.7°, 17.8°, 18.9°, and 21.2°°, and one, two or three of the degree 2θ-reflections (+/−0.2 degrees 2θ) at 6.3°, 28.0°, and 28.7°. In some embodiments, crystalline Formula I, Form II has an XRPD pattern comprising 2θ-reflections (+/−0.2 degrees 2θ) at 9.5°, 10.9°, 14.7°, 17.8°, 18.9°, and 21.2°°, and one or two of the degree 2θ-reflections (+/−0.2 degrees 2θ) at 6.3°, 28.0°, and 28.7°. In some embodiments, crystalline Formula I, Form II has an XRPD pattern comprising 29-reflections (+/−0.2 degrees 2θ) at 9.5°, 10.9°, 14.7°, 17.8°, 18.9°, and 21.2°°, and one of the degree 2θ-reflections (+/−0.2 degrees 2θ) at 6.3°, 28.0°, and 28.7°. In some embodiments, crystalline Formula I, Form II has an XRPD pattern comprising 2θ-reflections (+/−0.2 degrees 2θ) at 9.5°, 10.9°, 14.7°, 17.82, 18.9°, and 21.2°%, and two of the degree 2θ-reflections (+/−0.2 degrees 2θ) at 6.3°, 28.0°, and 28.7°. In some embodiments, crystalline Formula I, Form II has an XRPD pattern comprising any three of the degree 2θ-reflections (+/−0.2 degrees 2θ) selected from the group consisting of 6.3°. 9.5°, 10.9°, 14.7°, 17.8°, 18.9°, 21.2°, 28.0°, and 28.7°. In some embodiments, crystalline Formula I, Form II has an XRPD pattern comprising 2θ-reflections (+/−0.2 degrees 2θ) at 6.3°, 9.5°, 10.9°, 14.7°, 17.8°, 18.9°, 21.2°, 28.0°, and 28.7°. In some embodiments, crystalline Formula I, Form II has an XRPD pattern comprising at least three 2θ-reflections (+/−0.2 degrees 2θ) at 5.5, 6.3°, 8.9°, 9.5°, 10.9°, 14.7°, 15.7°, 16.3°, 17.3°, 17.8°, 18.0°, 18.9°, 19.5°, 21.2°, 28.0°, and 28.7°. In some embodiments, crystalline Formula I, Form II has an XRPD pattern comprising at least four 2θ-reflections (+/−0.2 degrees 2θ) at 5.5, 6.3°, 8.9°, 9.5°, 10.9°, 14.7°, 15.7°, 16.3°, 17.3°, 17.8°, 18.0°, 18.9°, 19.5°, 21.2°, 28.0°, and 28.7°. In some embodiments, crystalline Formula I. Form II has an XRPD pattern comprising at least five 2θ-reflections (+/−0.2 degrees 2θ) at 5.5, 6.3°, 8.9°, 9.5°, 10.9°, 14.7°, 15.7°, 16.3°, 17.3°, 17.8°, 18.0°, 18.9°, 19.5°, 21.2°, 28.0°, and 28.7°.

In some embodiments, Formula I, Form II has an XRPD pattern comprising peaks at:

| Pos. [°2Th. ± 0.2] | Rel. Int. [%] |
|---|---|
| 5.4610 | 17.91 |
| 6.3322 | 25.41 |
| 8.9092 | 23.95 |
| 9.4542 | 100.00 |
| 10.9298 | 69.05 |
| 13.2035 | 4.15 |
| 14.7383 | 62.27 |
| 15.6982 | 15.85 |
| 16.3292 | 15.33 |
| 17.2677 | 16.89 |
| 17.7594 | 48.53 |
| 18.0435 | 39.97 |
| 18.8815 | 55.12 |
| 19.4545 | 19.10 |
| 21.2189 | 64.10 |
| 23.1999 | 32.03 |
| 23.9297 | 32.13 |
| 25.7520 | 21.42 |
| 27.0226 | 14.90 |
| 27.4848 | 12.72 |
| 28.0375 | 43.04 |
| 28.6660 | 40.58 |
| 29.4663 | 20.64 |
| 30.1798 | 8.24 |
| 30.6728 | 18.29 |

In some embodiments, Formula I, Form II is solvated. In some embodiments, Formula I, Form II is partially or fully solvated. In some embodiments, Formula I, Form II is methanol solvated.

### Compound of Formula I, Form III

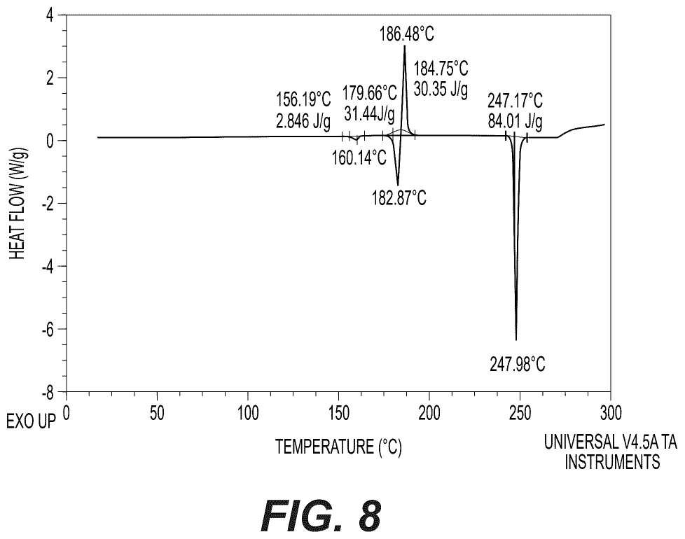

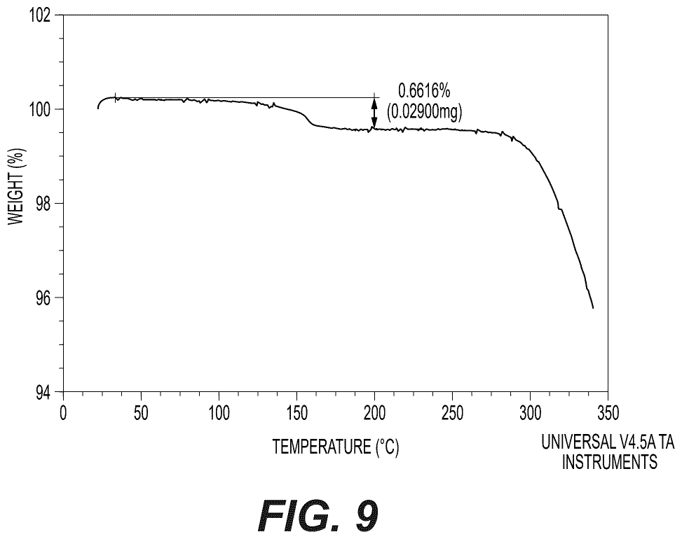

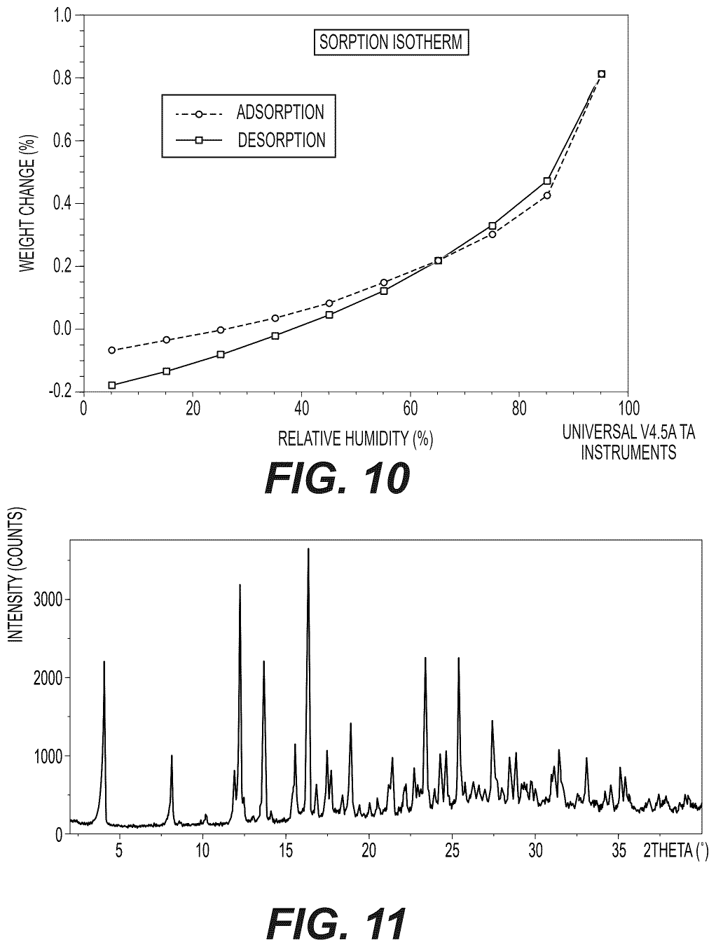

In some embodiments, provided is crystalline Form III of the compound of Formula I (Formula I, Form III). In some embodiments, Formula I, Form III is solvated. In some embodiments, Formula I, Form III is methanol solvated. In some embodiments, Formula I, Form III exhibits an XRPD pattern substantially as shown in FIG. 7. In some embodiments, Formula I, Form III exhibits a DSC thermogram substantially as shown in FIG. 8. In some embodiments, Formula I, Form III exhibits a TGA curve substantially as shown in FIG. 9. In some embodiments, Formula I, Form III exhibits a DVS graph substantially as shown in FIG. 10.

In some embodiments of Formula I, Form III, at least one, at least two, at least three, or all of the following (a)-(d) apply: (a) Formula I, Form III has an XRPD pattern substantially as shown in FIG. 7; (b) Formula I, Form III has a DSC thermogram substantially as shown in FIG. 8; (c) Formula I, Form III has a TGA curve substantially as shown in FIG. 9; (d) Formula I, Form III has a DVS curve substantially as shown in FIG. 10.

In some embodiments, Formula I, Form III has the following properties:
(a) an XRPD pattern substantially as shown in FIG. 7;
(b) a DSC thermogram substantially as shown in FIG. 8;
(c) a TGA curve substantially as shown in FIG. 9; and
(d) a DVS curve substantially as shown in FIG. 10.

In some embodiments, crystalline Formula I, Form III has an XRPD pattern displaying at least two, at least three, at least four, at least five, or at least six of the degree 2θ-reflections with the greatest intensity as the XRPD pattern substantially as shown in FIG. 7.

In some embodiments, crystalline Formula I, Form III has an XRPD pattern comprising degree 2θ-reflections (+/−0.2 degrees 2θ) at 15.8°, 17.3°, and 26.4°. In some embodiments, crystalline Formula I, Form III has an XRPD pattern comprising degree 2θ-reflections (+/−0.2 degrees 2θ) at 15.8°, 17.3°, and 26.4°, and one, two or three of the degree 2θ-reflections (+/−0.2 degrees 2θ) at 14.9°, 18.1°, and 21.8°. In some embodiments, crystalline Formula I, Form III has an XRPD pattern comprising degree 2θ-reflections (+/−0.2 degrees 2θ) at 15.8°, 17.3°, and 26.4°, and one or two of the degree 2θ-reflections (+/−0.2 degrees 2θ) at 14.9°, 18.1°, and 21.8°. In some embodiments, crystalline Formula I, Form III has an XRPD pattern comprising degree 2θ-reflections (+/−0.2 degrees 2θ) at 15.8°, 17.3°, and 26.4°, and one of the degree 2θ-reflections (+/−0.2 degrees 2θ) at 14.9°, 18.1°, and 21.89. In some embodiments, crystalline Formula I, Form III has an XRPD pattern comprising degree 2θ-reflections (+/−0.2 degrees 2θ) at 15.8°, 17.3°, and 26.4°, and two of the degree 29-reflections (+/−0.2 degrees 2θ) at 14.9°, 18.1°, and 21.8°. In some embodiments, crystalline Formula I, Form III has an XRPD pattern comprising degree 2θ-reflections (+/−0.2 degrees 2θ) at 14.9°, 15.8°, 17.39, 18.12, 21.8°, and 26.4°. In some embodiments, crystalline Formula I, Form III has an XRPD pattern comprising any three of the degree 2θ-reflections (+/−0.2 degrees 2θ) at 14.9°, 15.8°, 17.3°, 18.1°, 21.8°, and 26.4°.

In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°, and one, two or three of the degree 2θ-reflections (+/−0.2 degrees 2θ) at 5.8°. 9.7″, and 10.5°. In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°, and one or two of the degree 2θ-reflections (+/−0.2 degrees 2θ) at 5.8°, 9.7°, and 10.5°. In some embodiments, crystalline Formula I, Form III has an XRPD pattern comprising degree 2θ-reflections (+/−0.2 degrees 2θ) at 14.9°, 15.8°, 17.3°, 18.1°, 21.8°, and 26.4°, and one of the degree 29-reflections (+/−0.2 degrees 2θ) at 5.8°, 9.7°, and 10.5°. In some embodiments, crystalline Formula I, Form Ill has an XRPD pattern comprising degree 2θ-reflections (+/−0.2 degrees 2θ) at 14.9°, 15.8°, 17.3°, 18.1°, 21.8°, and 26.4°, and two of the degree 2θ-reflections (+/−0.2 degrees 2θ) at 5.8°, 9.7°, and 10.5°. In some embodiments, crystalline Formula I, Form III has an XRPD pattern comprising degree 2θ-reflections (+/−0.2 degrees 2θ) at 5.8°, 9.7°, 10.5°, 14.9°, 15.8°, 17.3°, 18.1°, 21.8°, and 26.4°. In some embodiments, crystalline Formula I, Form III has an XRPD pattern comprising any three of the degree 2θ-reflections (+/−0.2 degrees 2θ) at 5.8°, 9.7°, 10.5°, 14.9°, 15.8°, 17.3°, 18.1°, 21.8°, and 26.4°. In some embodiments, Formula I, Form III has an XRPD pattern comprising at least three of the degree 2θ-reflections (+/−0.2 degrees 2θ) at 5.8°, 9.7°, 10.5°, 11.5°, 14.9°, 15.8°, 17.3°, 18.1°, 21.8°, 26.4°, and 30.2°. In some embodiments, Formula I, Form III has an XRPD pattern comprising at least four of the degree 26-reflections (+/−0.2 degrees 2θ) at 5.8°, 9.7°, 10.5°, 11.5°, 14.9°, 15.8°, 17.3°, 18.1°, 21.8°, 26.4°, and 30.2°. In some embodiments, Formula I, Form III has an XRPD pattern comprising at least five of the degree 2θ-reflections (+/−0.2 degrees 2θ) at 5.8°, 9.7°, 10.5°, 11.5°, 14.9°, 15.82, 17.3°, 18.1°, 21.8°, 26.4°, and 30.2°.

In some embodiments, Formula I, Form III has an XRPD pattern comprising peaks at:

| Pos. [°2Th. ± 0.2] | Rel. Int. [%] |
|---|---|
| 5.7681 | 24.93 |
| 9.7205 | 22.56 |
| 10.5053 | 40.02 |
| 11.5440 | 29.19 |
| 14.9183 | 53.73 |
| 15.8074 | 64.25 |
| 17.2791 | 100.00 |
| 18.0835 | 51.41 |
| 19.4465 | 36.71 |
| 21.7763 | 55.41 |
| 23.7336 | 8.60 |
| 24.7317 | 5.43 |
| 26.3864 | 88.10 |
| 29.4755 | 23.36 |
| 30.2145 | 37.76 |
| 30.7309 | 18.01 |

In some embodiments, Formula I, Form III is characterized by a DSC thermogram substantially as shown in FIG. 8.

In some embodiments, Formula I, Form III is characterized by a DSC thermogram having one, two, three, or all of (i) an endothermic transition at about 156° C., (ii) an endothermic transition at about 180° C., (iii) an exothermic transition at about 186° C., and (iv) an endothermic transition at about 247° C.

In some embodiments, Formula I, Form III is characterized by a TGA curve substantially as shown in FIG. 9.

In some embodiments, Formula I, Form III is characterized by a DVS curve substantially as shown in shown in FIG. 10.

In some embodiments, Formula I, Form III is solvated. In some embodiments, Formula I, Form III is partially or fully solvated. In some embodiments, Formula I, Form III is methanol solvated.

### Compound of Formula I, Form IV

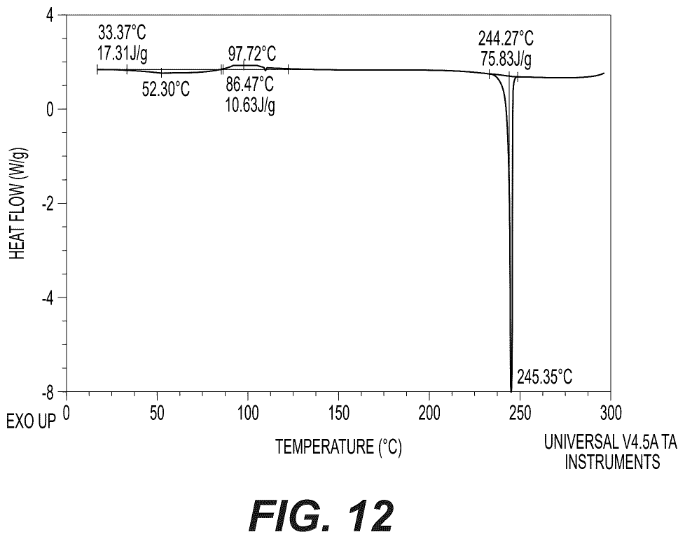

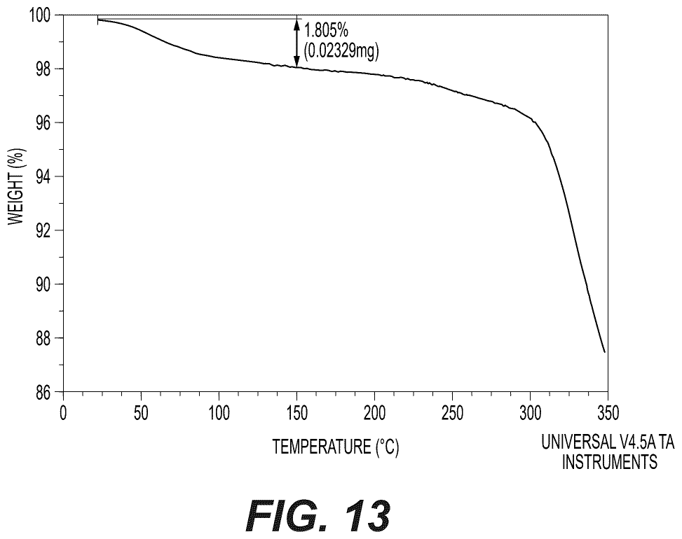

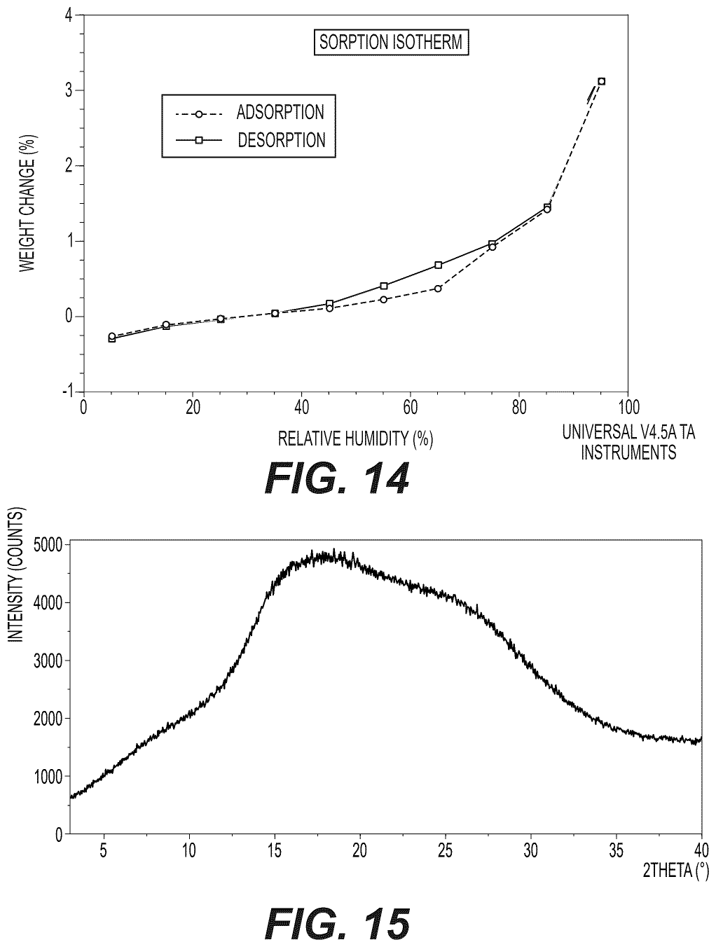

In some embodiments, provided is crystalline Form IV of the compound of Formula I (Formula I, Form IV). In some embodiments, Formula I, Form IV is solvated. In some embodiments, Formula I, Form IV is hydrated. In some embodiments, Formula I, Form IV exhibits an XRPD pattern substantially as shown in FIG. 11. In some embodiments, Formula I, Form IV exhibits a DSC thermogram substantially as shown in FIG. 12. In some embodiments, Formula I, Form IV exhibits a TGA curve substantially as shown in FIG. 13. In some embodiments, Formula I, Form IV exhibits a DVS graph substantially as shown in FIG. 14.

In some embodiments of Formula I, Form IV, at least one, at least two, at least three, or all of the following (a)-(d) apply: (a) Formula I, Form IV has an XRPD pattern substantially as shown in FIG. 11; (b) Formula I, Form IV has a DSC thermogram substantially as shown in FIG. 12; (c) Formula I, Form IV has a TGA curve substantially as shown in FIG. 13; (d) Formula I, Form IV has a DVS curve substantially as shown in FIG. 14.

In some embodiments, Formula I, Form IV has the following properties:
(a) an XRPD pattern substantially as shown in FIG. 11;
(b) a DSC thermogram substantially as shown in FIG. 12;
(c) a TGA curve substantially as shown in FIG. 13; and
(d) a DVS curve substantially as shown in FIG. 14.

In some embodiments, crystalline Formula I, Form IV has an XRPD pattern displaying at least two, at least three, at least four, at least five, or at least six of the degree 2θ-reflections with the greatest intensity as the XRPD pattern substantially as shown in FIG. 11.

In some embodiments, crystalline Formula I, Form IV has an XRPD pattern comprising degree 2θ-reflections (+/−0.2 degrees 2θ) at 4.10, 12.30, and 16.4°. In some embodiments, crystalline Formula I, Form IV has an XRPD pattern comprising degree 2θ-reflections (+/−0.2 degrees 2θ) at 4.1°, 12.3°, and 16.4°, and one, two or three of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, and 16.4°, and one or two of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, and 16.4°, and one of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, and 16.4°, and two of the degree 2θ-reflections (+/−0.2 degrees 2θ) at 13.7°, 23.4°, and 25.4°. In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°. In some embodiments, crystalline Formula I, Form IV has an XRPD pattern comprising any three of the degree 2θ-reflections (+/−0.2 degrees 2θ) at 4.1°, 12.3°, 13.7°, 16.4°, 23.4°, and 25.4°.

In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°, and one, two or three of the degree 2θ-reflections (+/−0.2 degrees 2θ) at 8.1°, 18.9°, and 27.4°. In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°, and one or two of the degree 2θ-reflections (+/−0.2 degrees 2θ) at 8.1°, 18.9°, and 27.4°. In some embodiments, crystalline Formula I, Form IV has an XRPD pattern comprising degree 29-reflections (+/−0.2 degrees 2θ) at 4.1°, 12.3°, 13.7°, 16.4°, 23.4°, and 25.4°, and one of the degree 2θ-reflections (+/−0.2 degrees 2θ) at 8.1°, 18.9°, and 27.4°. In some embodiments, crystalline Formula I, Form IV has an XRPD pattern comprising degree 2θ-reflections (+/−0.2 degrees 2θ) at 4.1°, 12.3°, 13.7°, 16.4°, 23.4°, and 25.4°, and two of the degree 2θ-reflections (+/−0.2 degrees 2θ) at 8.1°, 18.9°, and 27.4°. In some embodiments, crystalline Formula I, Form IV has an XRPD pattern comprising degree 2θ-reflections (+/−0.2 degrees 2θ) at 4.1°, 8.1°, 12.3°, 13.7°, 16.4°, 18.9°, 23.4°, 25.4°, and 27.4°. In some embodiments, crystalline Formula I, Form IV has an XRPD pattern comprising any three of the degree 2θ-reflections (+/−0.2 degrees 2θ) at 4.1°, 8.19, 12.3°, 13.7°, 16.4°, 18.9°, 23.4°, 25.4°, and 27.4°. In some embodiments, Formula I, Form IV has an XRPD pattern comprising at least three of the degree 2θ-reflections (+/−0.2 degrees 2θ) at 4.1°, 8.1°, 12.3°, 13.7°, 15.6°, 16.4°, 18.9°, 23.4°, 25.4°, and 27.4°. In some embodiments, Formula I, Form IV has an XRPD pattern comprising at least four of the degree 2θ-reflections (+/−0.2 degrees 2θ) at 4.1°, 8.1°, 12.3°, 13.7°, 15.6°, 16.4°, 18.9°, 23.4°, 25.4°, and 27.4°. In some embodiments, Formula I, Form IV has an XRPD pattern comprising at least five of the degree 2θ-reflections (+/−0.2 degrees 2θ) at 4.1°, 8.1°, 12.3°, 13.7°, 15.6°, 16.4°, 18.9°, 23.4°, 25.4°, and 27.4°.

In some embodiments, Formula I, Form IV has an XRPD pattern comprising peaks at:

| Pos. | Rel. Int. |
|---|---|
| [°2Th. ± 0.2] | [%] |
| 4.0905 | 61.21 |
| 8.1684 | 25.79 |
| 12.2546 | 85.48 |
| 13.7183 | 59.27 |
| 15.5882 | 25.92 |
| 16.3620 | 100.00 |
| 17.4922 | 23.39 |
| 18.4025 | 6.79 |
| 18.9112 | 34.07 |
| 21.3775 | 18.03 |
| 22.1915 | 9.88 |
| 23.3990 | 54.83 |
| 24.6191 | 18.60 |
| 25.4026 | 52.81 |
| 27.4476 | 29.32 |
| 28.4581 | 15.08 |
| 28.8361 | 16.20 |
| 31.4865 | 14.90 |
| 33.0815 | 17.71 |
| 35.1201 | 13.63 |

In some embodiments, Formula I, Form IV is characterized by a DSC thermogram substantially as shown in FIG. 12.

In some embodiments, Formula I, Form IV is characterized by a DSC thermogram having one, two or all of (i) an endothermic transition at about 33° C., (ii) an exothermic transition at about 86° C., and (iii) an endothermic transition at about 244° C.

In some embodiments, Formula I, Form IV is characterized by a TGA curve substantially as shown in FIG. 13.

In some embodiments, Formula I, Form IV is characterized by a DVS curve substantially as shown in shown in FIG. 14.

In some embodiments, Formula I, Form IV is hydrated. In some embodiments, Formula I, Form IV is partially or fully hydrated.

### Compound of Formula I, Amorphous

In some embodiments, the compound of Formula I is amorphous (Formula I, amorphous). In some embodiments, Formula I, amorphous has an XRPD profile substantially as shown in FIG. 15.

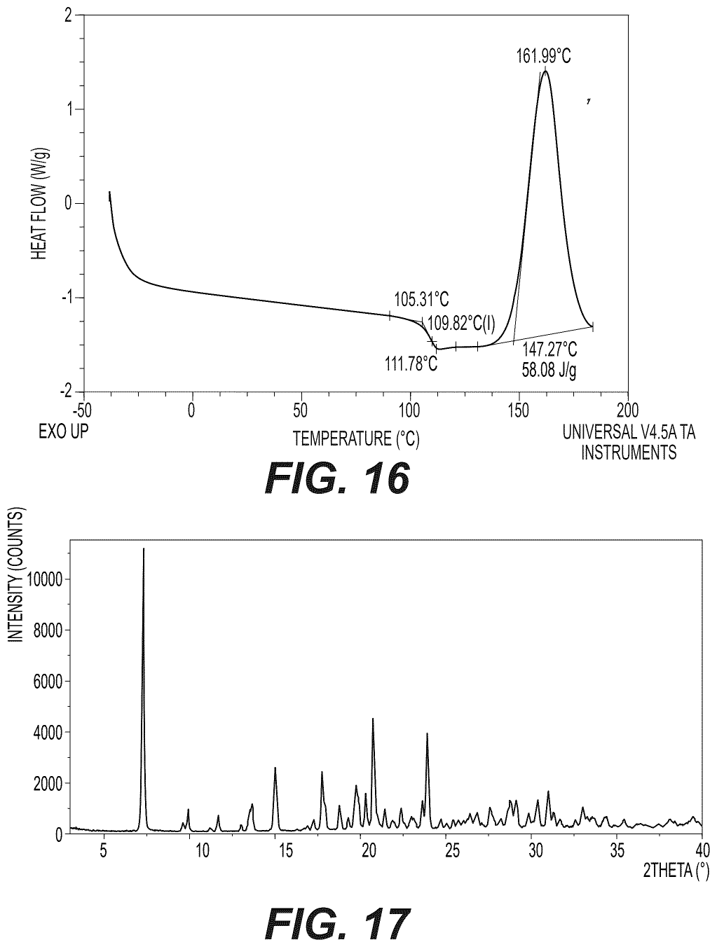

In some embodiments, Formula I, amorphous is characterized by a DSC thermogram substantially as shown in FIG. 16.

In some embodiments, Formula I, calcium salt, amorphous is characterized by a DSC thermogram having a glass transition about 110° C.

### Compound of Formula I, Sodium Salt

In some embodiments, the compound of Formula I can be isolated as a sodium salt which can be amorphous or crystalline. In some embodiments, the sodium salt of the compound of Formula I is crystalline.

In some embodiments, the crystalline sodium salt of the compound of Formula I has an XRPD profile substantially as shown in FIG. 17.

In some embodiments, the crystalline sodium salt of the compound of Formula I has an XRPD pattern comprising degree 2θ-reflections (+/−0.2 degrees 2θ) at 6.5°, 20.2°, and 23.5°. In some embodiments, the crystalline sodium salt of the compound of Formula I has an XRPD pattern comprising degree 2θ-reflections (+/−0.2 degrees 2θ) at 6.5°, 20.2°, and 23.5°, and one, two or three of the degree 2θ-reflections (+/−0.2 degrees 2θ) at 14.3°, 17.2°, and 19.2°. In some embodiments, the crystalline sodium salt of the compound of Formula I has an XRPD pattern comprising degree 2θ-reflections (+/−0.2 degrees 2θ) at 6.5°, 20.2°, and 23.5°, and one or two of the degree 2θ-reflections (+/−0.2 degrees 2θ) at 14.3°, 17.2°, and 19.2°. In some embodiments, the crystalline sodium salt of the compound of Formula I has an XRPD pattern comprising degree 2θ-reflections (+/−0.2 degrees 2θ) at 6.5°, 20.2°, and 23.5°, and one of the degree 2θ-reflections (+/−0.2 degrees 2θ) at 14.3°, 17.2°, and 19.2°. In some embodiments, the crystalline sodium salt of the compound of Formula I has an XRPD pattern comprising degree 2θ-reflections (+/−0.2 degrees 2θ) at 6.5°, 20.2°, and 23.5°, and two of the degree 2θ-reflections (+/−0.2 degrees 2θ) at 14.3°, 17.2°, and 19.2°. In some embodiments, the crystalline sodium salt of the compound of Formula I has an XRPD pattern comprising degree 29-reflections (+/−0.2 degrees 2θ) at 6.5°, 14.3°, 17.2°, 19.2°, 20.2°, and 23.5°. In some embodiments, the crystalline sodium salt of the compound of Formula I has an XRPD pattern comprising any three of the degree 2θ-reflections (+/−0.2 degrees 2θ) at 6.5°, 14.3°, 17.2°, 19.2°, 20.2°, and 23.59.

In some embodiments, the crystalline sodium salt of the compound of Formula I has an XRPD pattern comprising degree 2θ-reflections (+/−0.2 degrees 2θ) at 6.5°, 14.3°, 17.2°, 19.2°, 20.2°, and 23.5°, and one, two or three of the degree 2θ-reflections (+/−0.2 degrees 2θ) at 13.0°, 19.8°, and 30.8°. In some embodiments, the crystalline sodium salt of the compound of Formula I has an XRPD pattern comprising degree 2θ-reflections (+/−0.2 degrees 2θ) at 6.5°, 14.3°, 17.2°, 19.2°, 20.2°, and 23.5°, and one or two of the degree 2θ-reflections (+/−0.2 degrees 2θ) at 13.0°, 19.8°, and 30.8°. In some embodiments, the crystalline sodium salt of the compound of Formula I has an XRPD pattern comprising degree 2θ-reflections (+/−0.2 degrees 2θ) at 6.5°, 14.3°, 17.2°, 19.2°, 20.2°, and 23.5°, and one of the degree 2θ-reflections (+/−0.2 degrees 2θ) at 13.0°, 19.8°, and 30.8°. In some embodiments, the crystalline sodium salt of the compound of Formula I has an XRPD pattern comprising degree 2θ-reflections (+/−0.2 degrees 2θ) at 6.5°, 14.3°, 17.2°, 19.2°, 20.2°, and 23.5°, and two of the degree 2θ-reflections (+/−0.2 degrees 2θ) at 13.0°, 19.8°, and 30.8°. In some embodiments, the crystalline sodium salt of the compound of Formula I has an XRPD pattern comprising degree 2θ-reflections (+/−0.2 degrees 2θ) at 6.5°, 13.0° 14.3°, 17.2°, 19.2°, 19.8°, 20.2°, 23.5°, and 30.8°. In some embodiments, the crystalline sodium salt of the compound of Formula I has an XRPD pattern comprising any three of the degree 2θ-reflections (+/−0.2 degrees 2θ) at 6.5°, 13.0° 14.3°, 17.2°, 19.2°, 19.8°, 20.2°, 23.5°, and 30.8°.

In some embodiments, the crystalline sodium salt of the compound of Formula I has an XRPD pattern comprising peaks at:

| Pos. | Rel. Int. |
|---|---|
| [°2Th. ± 0.2] | [%] |
| 6.4582 | 100.00 |
| 9.1357 | 7.98 |
| 10.9197 | 5.37 |
| 12.9859 | 9.48 |
| 14.3397 | 23.23 |
| 16.6524 | 3.41 |
| 17.1777 | 21.24 |
| 18.2116 | 8.54 |
| 18.7336 | 3.89 |
| 19.2149 | 15.37 |
| 19.7692 | 11.99 |
| 20.2415 | 39.53 |
| 20.9268 | 6.60 |
| 21.8961 | 6.32 |
| 22.5979 | 3.26 |
| 23.1770 | 9.59 |
| 23.4848 | 34.81 |
| 26.4924 | 4.82 |
| 27.2889 | 6.52 |
| 28.4243 | 7.38 |
| 28.8215 | 8.79 |
| 30.1002 | 9.22 |
| 30.7739 | 11.53 |
| 32.8031 | 6.89 |
| 34.1696 | 3.24 |

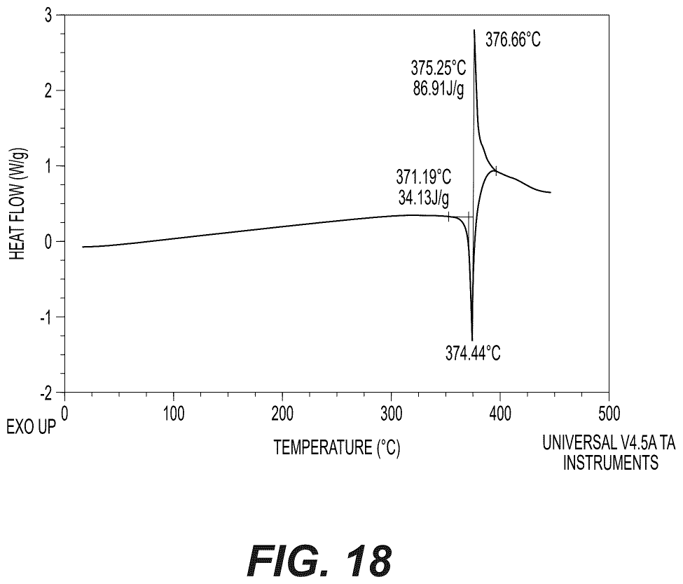

In some embodiments, the crystalline sodium salt of the compound of Formula I is characterized by a DSC thermogram substantially as shown in FIG. 18.

In some embodiments, the crystalline sodium salt of the compound of Formula I is characterized by a DSC thermogram having one or both of (i) an endothermic transition at about 371° C. and (ii) an exothermic transition at about 374° C.

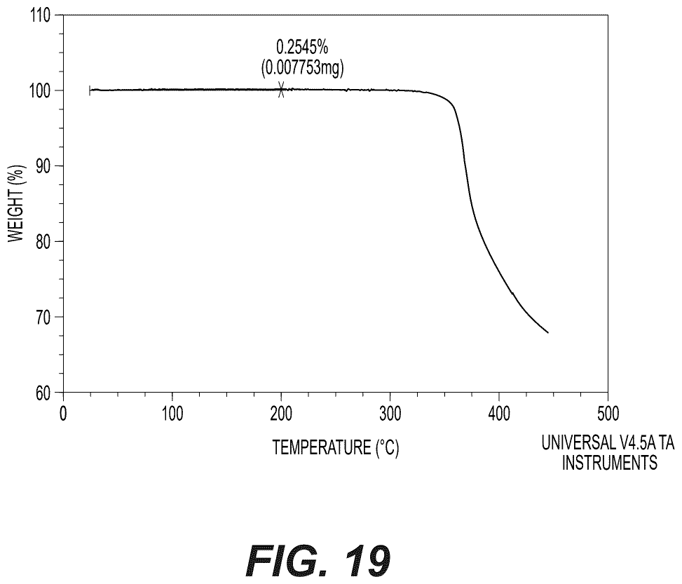

In some embodiments, the crystalline sodium salt of the compound of Formula I is characterized by a TGA curve substantially as shown in FIG. 19.

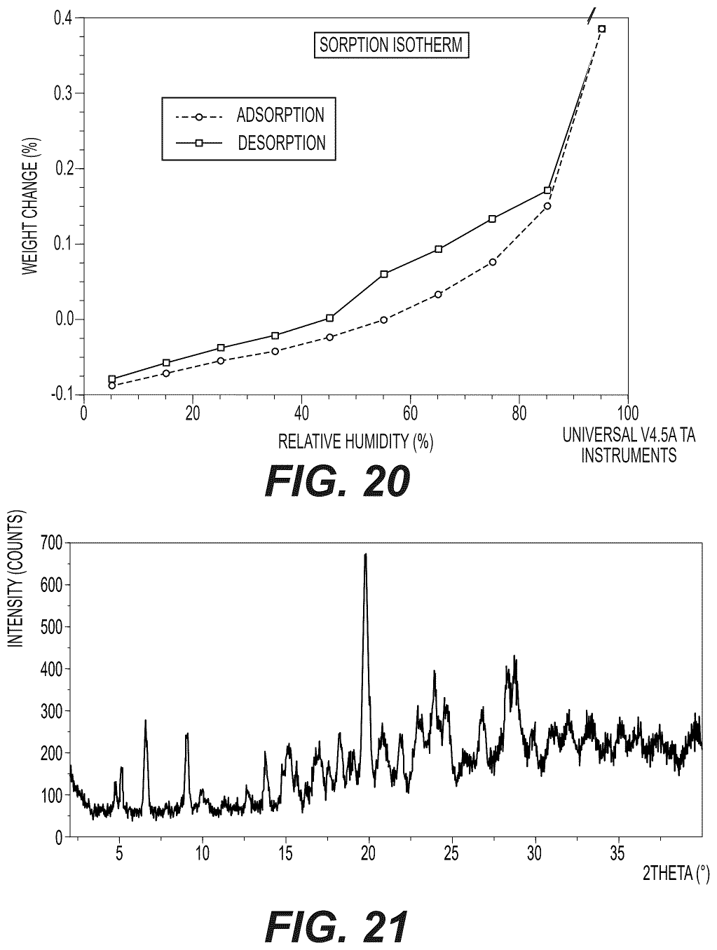

In some embodiments, the crystalline sodium salt of the compound of Formula I is characterized by a DVS curve substantially as shown in shown in FIG. 20.

In some embodiments, the crystalline sodium salt of the compound of Formula I has the following properties:
(a) an XRPD) pattern substantially as shown in FIG. 17;
(b) a DSC thermogram substantially as shown in FIG. 18;
(c) a TGA curve substantially as shown in FIG. 19; and
(d) a DVS curve substantially as shown in FIG. 20.

In some embodiments, the crystalline sodium salt of the compound of Formula I is anhydrous. In some embodiments, the crystalline sodium salt of the compound of Formula I is unsolvated.

### Compound of Formula I, Potassium Salt

In some embodiments, the compound of Formula I can be isolated as a potassium salt which can be amorphous or crystalline. In some embodiments, the potassium salt of the compound of Formula I is crystalline. In some embodiments, the crystalline form of the potassium salt of the compound of Formula I is hydrated.

In some embodiments, the crystalline potassium salt of the compound of Formula I is the Form I (Formula I, potassium salt, Form I). In some embodiments, Formula I, potassium salt, Form I is hydrated. In some embodiments. Formula I, potassium salt, Form I has an XRPD profile substantially as shown in FIG. 21.

In some embodiments, Formula I, potassium salt, Form I has an XRPD pattern comprising degree 2θ-reflections (+/−0.2 degrees 2θ) at 6.6°, 19.7°, and 23.9°. In some embodiments, Formula I, potassium salt, Form I has an XRPD pattern comprising degree 2θ-reflections (+/−0.2 degrees 2θ) at 6.6°, 19.7°, and 23.9°, and one, two or three of the degree 29-reflections (+/−0.2 degrees 2θ) at 9.0°, 28.3°, and 28.8°. In some embodiments, Formula I, potassium salt. Form I has an XRPD pattern comprising degree 2θ-reflections (+/−0.2 degrees 2θ) at 6.6°, 19.7°, and 23.9°, and one or two of the degree 2θ-reflections (+/−0.2 degrees 2θ) at 9.0°, 28.32, and 28.8°. In some embodiments, Formula I, potassium salt, Form I has an XRPD pattern comprising degree 2θ-reflections (+/−0.2 degrees 2θ) at 6.6°, 19.7°, and 23.9°, and one of the degree 29-reflections (+/−0.2 degrees 2θ) at 9.0°, 28.3°, and 28.8°. In some embodiments, Formula I, potassium salt, Form I has an XRPD pattern comprising degree 2θ-reflections (+/−0.2 degrees 2θ) at 6.6°, 19.7°, and 23.9°, and two of the degree 2θ-reflections (+/−0.2 degrees 2θ) at 9.0°, 28.3°, and 28.8°. In some embodiments, Formula I, potassium salt, Form I has an XRPD pattern comprising degree 2θ-reflections (+/−0.2 degrees 2θ) at 6.6°. 9.0°, 19.7°, 23.9°, 28.3°, and 28.8°. In some embodiments. Formula I, potassium salt. Form I has an XRPD pattern comprising any three of the degree 2θ-reflections (+/−0.2 degrees 2θ) at 6.6°, 9.0°, 19.7°, 23.9°, 28.3°, and 28.8°.

In some embodiments, Formula I, potassium salt, Form I has an XRPD pattern comprising degree 2θ-reflections (+/−0.2 degrees 2θ) at 6.6°, 9.0°, 19.7°, 23.9°, 28.3°, and 28.8°, and one, two or three of the degree 2θ-reflections (+/−0.2 degrees 2θ) at 15.2°, 22.92, and 24.7°. In some embodiments, Formula I, potassium salt, Form I has an XRPD pattern comprising degree 2θ-reflections (+/−0.2 degrees 2θ) at 6.6°, 9.0°, 19.7°, 23.9°, 28.3°, and 28.8°, and one or two of the degree 2θ-reflections (+/−0.2 degrees 2θ) at 15.2°, 22.9°, and 24.7″. In some embodiments, Formula I, potassium salt, Form I has an XRPD pattern comprising degree 2θ-reflections (+/−0.2 degrees 2θ) at 6.6°. 9.0°, 19.7°, 23.9°, 28.3°, and 28.8°, and one of the degree 2θ-reflections (+/−0.2 degrees 2θ) at 15.2°, 22.9°, and 24.7°. In some embodiments, Formula I, potassium salt, Form I has an XRPD pattern comprising degree 2θ-reflections (+/−0.2 degrees 20) at 6.6°, 9.0°, 19.7°, 23.9°, 28.3°, and 28.8°, and two of the degree 2θ-reflections (+/−0.2 degrees 2θ) at 15.2°, 22.9°, and 24.7°. In some embodiments, Formula I, potassium salt. Form I has an XRPD pattern comprising degree 2θ-reflections (+/−0.2 degrees 2θ) at 6.6°, 9.0°, 15.2°, 19.7°, 22.9°, 23.9°, 24.7°, 28.3°, and 28.8°. In some embodiments, Formula I, potassium salt, Form I has an XRPD pattern comprising any three of the degree 2θ-reflections (+/−0.2 degrees 2θ) at 6.6°, 9.0°, 15.2°, 19.7°, 22.9°, 23.9°, 24.7°, 28.3°, and 28.8°. In some embodiments, Formula I, potassium salt, Form I has an XRPD pattern comprising at least three of the degree 2θ-reflections (+/−0.2 degrees 2θ) at 5.1°, 6.6°, 9.0°, 13.8°, 15.2°, 19.7°, 22.9°, 23.9°, 24.7°, 28.3°, and 28.8°. In some embodiments, Formula I, potassium salt, Form I has an XRPD pattern comprising at least four of the degree 26-reflections (+/−0.2 degrees 2θ) at 5.1°, 6.6°, 9.0°, 13.8°, 15.2°, 19.7°, 22.9°, 23.9°, 24.7°, 28.3°, and 28.8°. In some embodiments, Formula I, potassium salt, Form I has an XRPD pattern comprising at least five of the degree 2θ-reflections (+/−0.2 degrees 2θ) at 5.1°, 6.6°, 9.0°, 13.8°, 15.2°, 19.7°, 22.9°, 23.9°, 24.7°, 28.3°, and 28.8°.

In some embodiments, Formula I, potassium salt, Form I has an XRPD pattern comprising peaks at:

| Pos. | Rel. Int. |
|---|---|
| [°2Th. ± 0.2] | [%] |
| 5.1492 | 21.58 |
| 6.5914 | 46.20 |
| 9.0435 | 37.69 |
| 10.0631 | 8.48 |
| 13.7803 | 24.04 |
| 15.2024 | 28.15 |
| 16.9249 | 19.94 |
| 18.2685 | 25.25 |
| 19.7188 | 100.00 |
| 20.7272 | 19.45 |
| 21.8640 | 20.74 |
| 22.9141 | 27.99 |
| 23.9132 | 46.53 |
| 24.6611 | 34.57 |
| 26.7792 | 25.53 |
| 28.2922 | 41.93 |
| 28.7775 | 45.47 |
| 29.8952 | 13.62 |
| 31.0161 | 13.16 |
| 32.0096 | 16.24 |
| 33.3248 | 13.97 |
| 35.1042 | 14.69 |

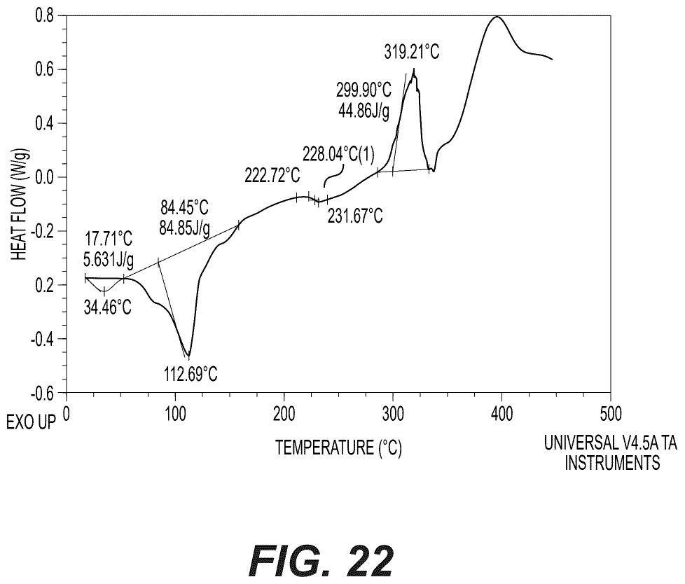

In some embodiments, Formula I, potassium salt, Form I is characterized by a DSC thermogram substantially as shown in FIG. 22.

In some embodiments, Formula I, potassium salt, Form I is characterized by a DSC thermogram having one, two, three, or all four of (i) an endothermic transition at about 18° C., (ii) an endothermic transition at about 84° C., (iii) a baseline shift at 228° C., and (iv) an exothermic transition at about 300° C.

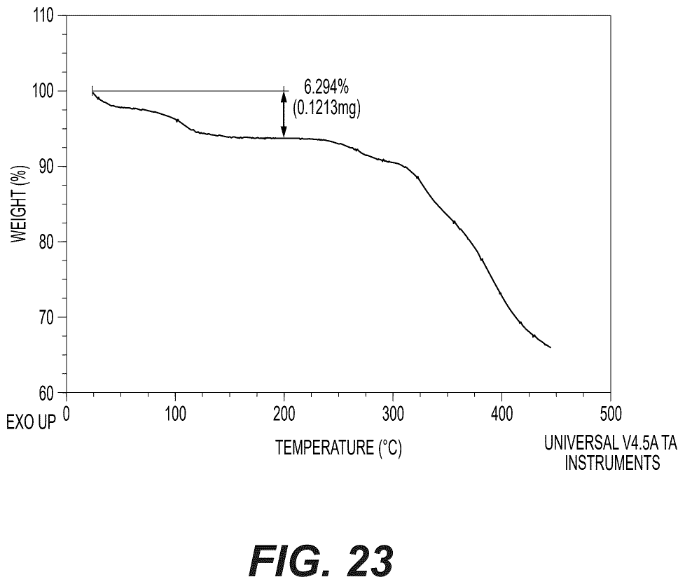

In some embodiments, Formula I, potassium salt, Form I is characterized by a TGA curve substantially as shown in FIG. 23.

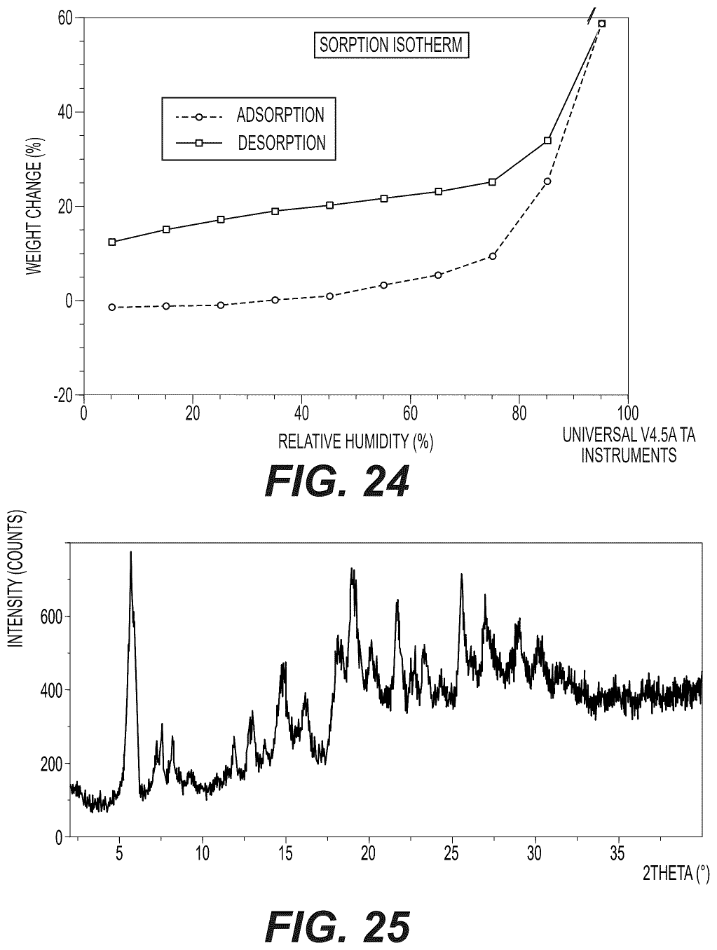

In some embodiments, Formula I, potassium salt, Form I is characterized by a DVS curve substantially as shown in shown in FIG. 24.

In some embodiments, Formula I, potassium salt, Form I has the following properties:
(a) an XRPD pattern substantially as shown in FIG. 21;
(b) a DSC thermogram substantially as shown in FIG. 22;
(c) a TOA curve substantially as shown in FIG. 23; and
(d) a DVS curve substantially as shown in FIG. 24.

In some embodiments, Formula I, potassium salt, Form I is hydrated. In some embodiments, Formula I, potassium salt, Form I is partially or fully hydrated.

In some embodiments, the crystalline potassium salt of the compound of Formula I is the Form II (Formula I, potassium salt, Form II). In some embodiments, Formula I, potassium salt, Form II is hydrated. In some embodiments, Formula I, potassium salt, Form II has an XRPD profile substantially as shown in FIG. 25.

In some embodiments, Formula I, potassium salt, Form II has an XRPD pattern comprising degree 2θ-reflections (+/−0.2 degrees 2θ) at 5.6°, 19.0°, and 25.6°. In some embodiments, Formula I, potassium salt, Form II has an XRPD pattern comprising degree 2θ-reflections (+/−0.2 degrees 2θ) at 5.6°, 19.0°, and 25.6°, and one, two or three of the degree 2θ-reflections (+/−0.2 degrees 2θ) at 14.9, 18.1, 21.7°. In some embodiments, Formula I, potassium salt, Form II has an XRPD pattern comprising degree 2θ-reflections (+/−0.2 degrees 2θ) at 5.6°, 19.0°, and 25.6°, and one or two of the degree 2θ-reflections (+/−0.2 degrees 2θ) at 14.9, 18.1, 21.7°. In some embodiments, Formula I, potassium salt, Form II has an XRPD pattern comprising degree 2θ-reflections (+/−0.2 degrees 2θ) at 5.6°, 19.0°, and 25.6°, and one of the degree 2θ-reflections (+/−0.2 degrees 2θ) at 14.9, 18.1, 21.7°. In some embodiments, Formula I, potassium salt, Form II has an XRPD pattern comprising degree 2θ-reflections (+/−0.2 degrees 2θ) at 5.6°, 19.0°, and 25.6°, and two of the degree 2θ-reflections (+/−0.2 degrees 2θ) at 14.9, 18.1, 21.7°. In some embodiments, Formula I, potassium salt, Form II has an XRPD pattern comprising degree 2θ-reflections (+/−0.2 degrees 2θ) at 5.6°, 14.9, 18.1, 19.0°, 21.7°, and 25.6°. In some embodiments. Formula I, potassium salt, Form II has an XRPD pattern comprising any three of the degree 2θ-reflections (+/−0.2 degrees 2θ) at 5.6°, 14.9, 18.1, 19.0°, 21.7°, and 25.6°.

In some embodiments, Formula I, potassium salt, Form II has an XRPD pattern comprising degree 2θ-reflections (+/−0.2 degrees 2θ) at 5.6°, 14.9, 18.1, 19.0°, 21.7°, and 25.6°, and one, two or three of the degree 2θ-reflections (+/−0.2 degrees 2θ) at 13.0°, 16.2°, and 27.0°. In some embodiments, Formula I, potassium salt. Form II has an XRPD pattern comprising degree 2θ-reflections (+/−0.2 degrees 2θ) at 5.6°, 14.9, 18.1, 19.0°, 21.7°, and 25.6°, and one or two of the degree 2θ-reflections (+/−0.2 degrees 2θ) at 13.0°, 16.2°, and 27.0°. In some embodiments, Formula I, potassium salt, Form II has an XRPD pattern comprising degree 2θ-reflections (+/−0.2 degrees 2θ) at 5.6°, 14.9, 18.1, 19.0°, 21.7°, and 25.6°, and one of the degree 2θ-reflections (+/−0.2 degrees 2θ) at 13.0°, 16.2°, and 27.0°. In some embodiments, Formula I, potassium salt, Form II has an XRPD pattern comprising degree 26-reflections (+/−0.2 degrees 2θ) at 5.6°, 14.9, 18.1, 19.0°, 21.7°, and 25.6°, and two of the degree 2θ-reflections (+/−0.2 degrees 2θ) at 13.0°, 16.2°, and 27.0°. In some embodiments, Formula I, potassium salt, Form II has an XRPD pattern comprising degree 2θ-reflections (+/−0.2 degrees 2θ) at 5.6°, 13.0°, 14.9, 16.2°, 18.1, 19.0°, 21.7°, 25.6°, and 27.0°. In some embodiments, Formula I, potassium salt, Form II has an XRPD pattern comprising any three of the degree 2θ-reflections (+/−0.2 degrees 2θ) at 5.6°, 13.0°, 14.9, 16.2°, 18.1, 19.0°, 21.7°, 25.6°, and 27.0°. In some embodiments, Formula I, potassium salt, Form II has an XRPD pattern comprising at least three of the degree 2θ-reflections (+/−0.2 degrees 2θ) at 5.6°. 7.4°, 13.0°, 14.9, 16.2°, 18.1, 19.0°, 21.7°, 25.6°, and 27.0°. In some embodiments, Formula I, potassium salt, Form II has an XRPD pattern comprising at least four of the degree 2θ-reflections (+/−0.2 degrees 2θ) at 5.6°, 7.4°, 13.0°, 14.9, 16.2°, 18.1, 19.0°, 21.7°, 25.6°, and 27.0°. In some embodiments, Formula I, potassium salt, Form II has an XRPD pattern comprising at least five of the degree 2θ-reflections (+/−0.2 degrees 2θ) at 5.6°, 7.4°, 13.0°, 14.9, 16.2°, 18.1, 19.0°, 21.7°, 25.6°, and 27.0°.

In some embodiments, Formula I, potassium salt, Form II has an XRPD pattern comprising peaks at:

| Pos. | Rel. Int. |
|---|---|
| [°2Th. ± 0.2] | [%] |
| 5.6498 | 100.00 |
| 7.3593 | 15.01 |
| 13.0013 | 20.94 |
| 14.8561 | 34.99 |
| 16.2062 | 22.04 |
| 18.1349 | 36.72 |
| 18.9590 | 63.84 |
| 21.7218 | 36.85 |
| 25.5562 | 46.28 |
| 26.9590 | 30.14 |
| 29.0217 | 18.72 |
| 30.1946 | 14.27 |

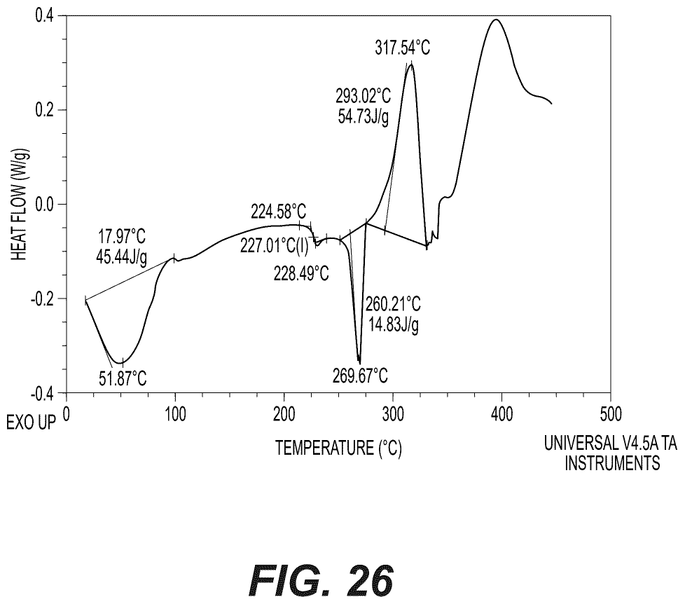

In some embodiments, Formula I, potassium salt, Form II is characterized by a DSC thermogram substantially as shown in FIG. 26.

In some embodiments, Formula I, potassium salt, Form II has the following properties:
(a) an XRPD pattern substantially as shown in FIG. 25; and
(b) a DSC thermogram substantially as shown in FIG. 26.

In some embodiments, Formula I, potassium salt, Form II is characterized by a DSC thermogram having one, two, three, or all four of (i) displays an endothermic transition at about 18° C., (ii) a baseline shift at 227°, (iii) an endothermic transition at about 260° C., and (iv) an exothermic transition at about 293° C.

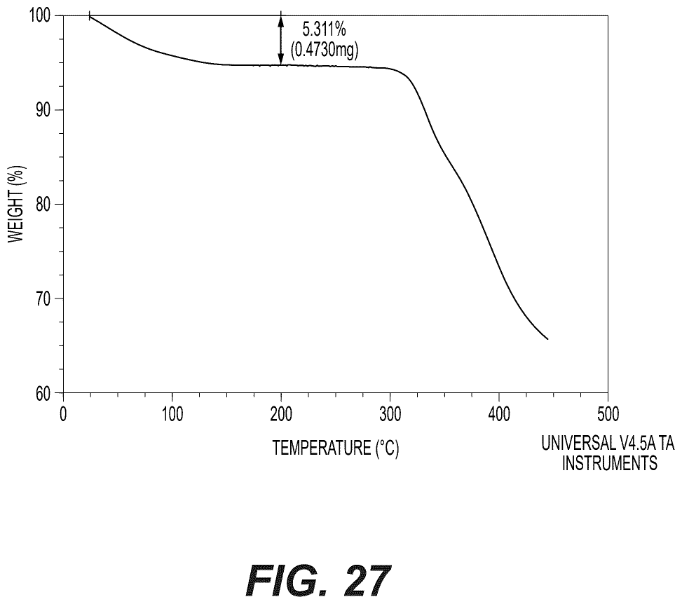

In some embodiments, Formula I, potassium salt, Form II is characterized by a TGA curve substantially as shown in FIG. 27.

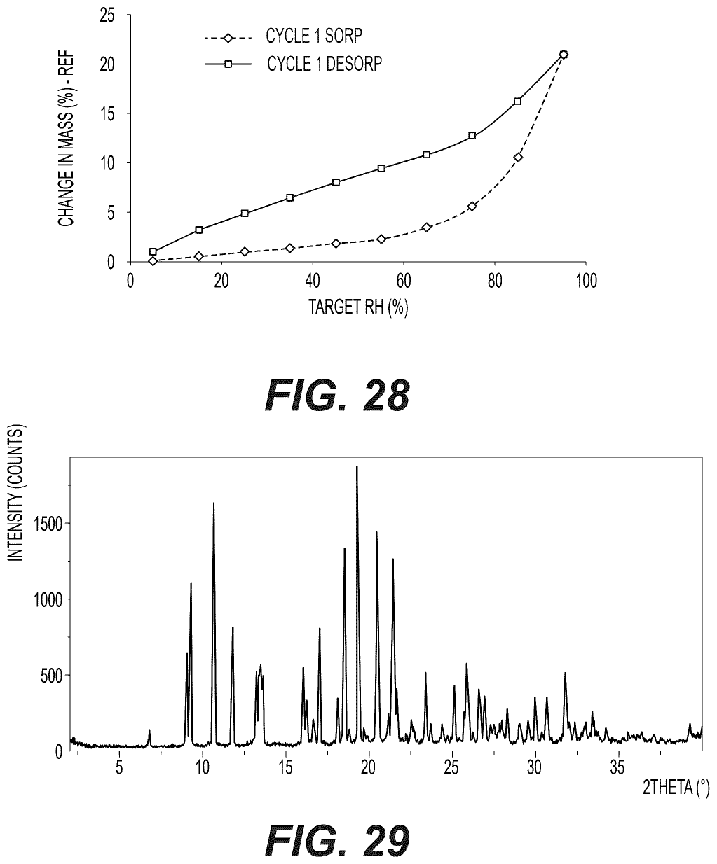

In some embodiments, Formula I, potassium salt, Form II is characterized by a DVS curve substantially as shown in shown in FIG. 28.

In some embodiments, Formula I, potassium salt, Form II is hydrated. In some embodiments, Formula I, potassium salt, Form II is partially or fully hydrated.

### Compound of Formula I, Calcium Salt

In some embodiments, the compound of Formula I can be isolated as a calcium salt which can be in any morphological form such as, for example, crystalline or amorphous, as well as disordered crystals, liquid crystals, plastic crystals, mesophases, and the like, or any combination thereof. In some embodiments, the calcium salt of the compound of Formula I is crystalline. In some embodiments, the crystalline form of the calcium salt of the compound of Formula I is hydrated.

In some embodiments, the crystalline calcium salt of the compound of Formula I is the Form I (Formula I, calcium salt, Form I). In some embodiments, Formula I, calcium salt, Form I is hydrated. In some embodiments, Formula I, calcium salt, Form I has an XRPD profile substantially as shown in FIG. 41.

In some embodiments, Formula I, calcium salt, Form I has an XRPD pattern comprising degree 2θ-reflections (+/−0.2 degrees 2θ) at 4.8°, 5.6°, 6.1°, and 7.6°. In some embodiments, Formula I, calcium salt, Form I has an XRPD pattern comprising any three of the degree 2θ-reflections (+/−0.2 degrees 2θ) at 4.8°, 5.6°, 6.1°, and 7.6°.

In some embodiments, Formula I, calcium salt, Form I has an XRPD pattern comprising peaks at:

| Pos. | Rel. Int. |
|---|---|
| [°2Th. ± 0.2] | [%] |
| 4.759 | 32.23 |
| 5.5975 | 52.28 |
| 6.1208 | 100 |
| 7.6264 | 14.89 |

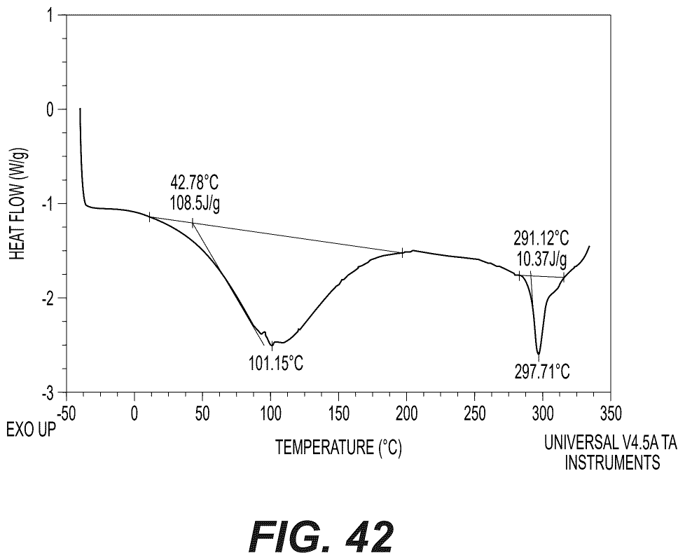

In some embodiments, Formula I, calcium salt, Form I is characterized by a DSC thermogram substantially as shown in FIG. 42.

In some embodiments, Formula I, calcium salt, Form I is characterized by a DSC thermogram having one or both of (i) an endothermic transition at about 43° C. and (ii) an endothermic transition at about 291° C. In some embodiments, Formula I, calcium salt, Form 1 is characterized by a DSC thermogram having an endothermic transition at about 43° C. and an endothermic transition at about 291° C.

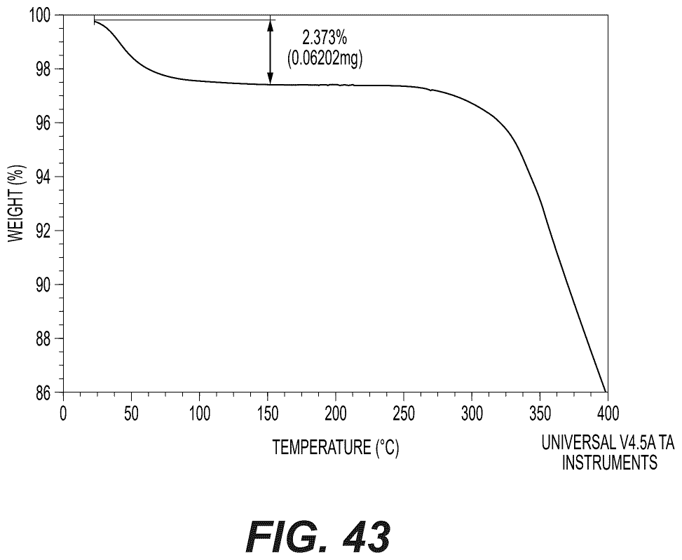

In some embodiments, Formula I, calcium salt, Form I is characterized by a TOGA curve substantially as shown in FIG. 43.

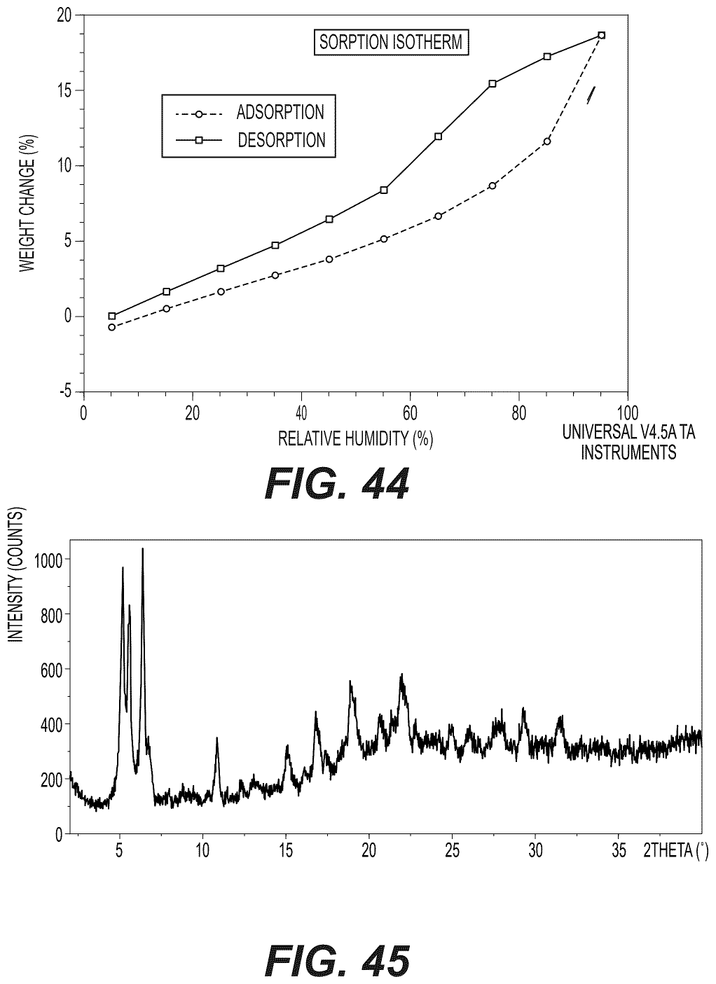

In some embodiments, Formula I, calcium salt, Form I is characterized by a DVS curve substantially as shown in shown in FIG. 44.

In some embodiments, Formula I, calcium salt, Form I has the following properties:
(a) an XRPD pattern substantially as shown in FIG. 41;
(b) a DSC thermogram substantially as shown in FIG. 42;
(c) a TGA curve substantially as shown in FIG. 43; and
(d) a DVS curve substantially as shown in FIG. 44.

In some embodiments, Formula I, calcium salt, Form I is hydrated. In some embodiments, Formula I, calcium salt, Form I is partially or fully hydrated.

In some embodiments, Formula I, calcium salt, Form I is a hemi-calcium salt.

In some embodiments, Formula I, calcium salt. Form I is present in a composition comprising a calcium salt of a halide, formate, acetate, triflate, or nitrate. In some embodiments, the calcium salt of a halide is a calcium salt of chloride, such as CaCl⁺ or CaCl₂.

In some embodiments, Formula I, calcium salt, Form I is present in a composition that also comprises about 0.1 to about 3.0 wt %, about 0.3 to about 2.5 wt %, about 0.4 to about 2.0 wt %, about 0.5 to about 1.0 wt %, about 0.6 to about 0.9 wt %, about 0.6 wt %, about 0.7 wt %, about 0.8 wt %, or about 0.9 wt % of chloride. In some embodiments, the chloride is in the form of a salt, such as a calcium salt like CaCl⁺ or CaCl₂.

In some embodiments, Formula I, calcium salt, Form I is present in a composition that also comprises the compound of Formula I in free form. In some embodiments, Formula I, calcium salt, Form I is present in a composition that also comprises chloride and the compound of Formula I in free form.

In some embodiments, the crystalline calcium salt of the compound of Formula I is the Form II (Formula I, calcium salt, Form II). In some embodiments, Formula I, calcium salt, Form II is hydrated. In some embodiments, Formula I, calcium salt, Form II has an XRPD profile substantially as shown in FIG. 45.

In some embodiments, Formula I, calcium salt, Form II has an XRPD pattern comprising degree 2θ-reflections (+/−0.2 degrees 2θ) at 5.2°, 5.6°, and 6.4°. In some embodiments, Formula I, calcium salt, Form II has an XRPD pattern comprising degree 2θ-reflections (+/−0.2 degrees 2θ) at 5.2°, 5.6°, and 6.4°, and one, two or three of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°. 5.6°, and 6.4°, and one or two of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°. 5.6°, and 6.4°, and one of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°. 5.6°, and 6.4°, and two of the degree 2θ-reflections (+/−0.2 degrees 2θ) at 10.9°, 18.9°, and 21.9°. In some embodiments, Formula I, calcium salt, Form II has an XRPD pattern comprising degree 2θ-reflections (+/−0.2 degrees 2θ) at 5.2°, 5.6°, 6.4°, 10.9°, 18.9°, and 21.9°. In some embodiments, Formula I, calcium salt, Form II has an XRPD pattern comprising any three of the degree 2θ-reflections (+/−0.2 degrees 2θ) at 5.2°, 5.6°, 6.4°, 10.9°, 18.9°, and 21.9°.

In some embodiments, Formula I, calcium salt, Form II has an XRPD pattern comprising degree 2θ-reflections (+/−0.2 degrees 2θ) at 5.2°, 5.6°, 6.4°, 10.9°, 18.9°, and 21.9°, and one, two or three of the degree 2θ-reflections (+/−0.2 degrees 2θ) at 15.1°, 16.8°, and 29.3°. In some embodiments, Formula I, calcium salt, Form II has an XRPD pattern comprising degree 2θ-reflections (+/−0.2 degrees 2θ) at 5.2°. 5.6°. 6.4°, 10.9°, 18.9°, and 21.9°, and one or two of the degree 2θ-reflections (+/−0.2 degrees 2θ) at 15.1°, 16.8°, and 29.3°. In some embodiments, Formula I, calcium salt, Form II has an XRPD pattern comprising degree 2θ-reflections (+/−0.2 degrees 2θ) at 5.2°, 5.6°, 6.4°, 10.92, 18.9°, and 21.92, and one of the degree 2θ-reflections (+/−0.2 degrees 2θ) at 15.1°, 16.8°, and 29.3°. In some embodiments, Formula I, calcium salt. Form II has an XRPD pattern comprising degree 2θ-reflections (+/−0.2 degrees 2θ) at 5.2°, 5.6°, 6.4°, 10.9°, 18.9°, and 21.9°, and two of the degree 2θ-reflections (+/−0.2 degrees 2θ) at 15.19, 16.8°, and 29.3°. In some embodiments, Formula I, calcium salt, Form II has an XRPD pattern comprising degree 2θ-reflections (+/−0.2 degrees 2θ) at 5.2°. 5.6°, 6.4°, 10.9°, 15.1°, 16.8°, 18.9°, 21.9°, and 29.3°. In some embodiments, Formula I, calcium salt, Form II has an XRPD pattern comprising any three of the degree 2θ-reflections (+/−0.2 degrees 2θ) at 5.2°. 5.6°, 6.4°, 10.9°, 15.1°, 16.8°, 18.9°, 21.9°, and 29.3°.

In some embodiments, Formula I, calcium salt, Form II has an XRPD pattern comprising peaks at:

| Pos. | Rel. Int. |
|---|---|
| [°2Th. ± 0.2] | [%] |
| 5.2027 | 94.25 |
| 5.6215 | 83.25 |
| 6.4645 | 100 |
| 10.8889 | 25.26 |
| 15.0964 | 15.92 |
| 16.8257 | 23.38 |
| 18.9083 | 29.87 |
| 21.9068 | 27.32 |
| 24.9566 | 6.72 |
| 25.9705 | 6.49 |
| 27.8323 | 9.87 |
| 29.2844 | 15.82 |
| 31.4693 | 12.42 |

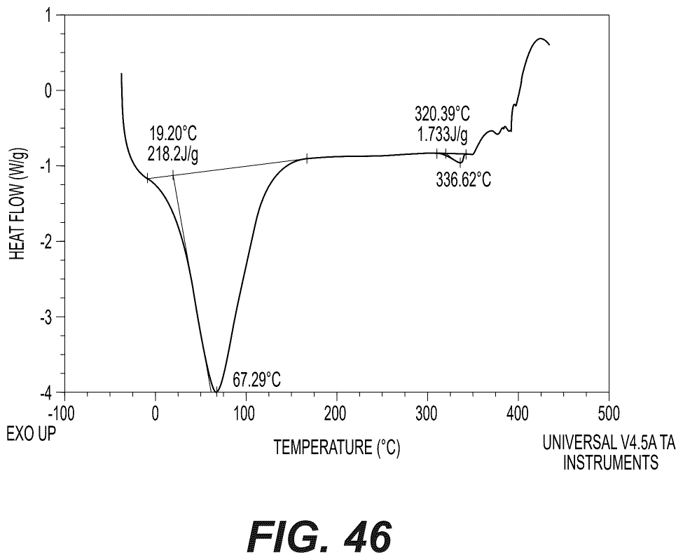

In some embodiments, Formula I, calcium salt, Form II is characterized by a DSC thermogram substantially as shown in FIG. 46.

In some embodiments, Formula I, calcium salt, Form II is characterized by a DSC thermogram having one or both of (i) displays an endothermic transition at about 19° C. and an endothermic transition at about 320° C.

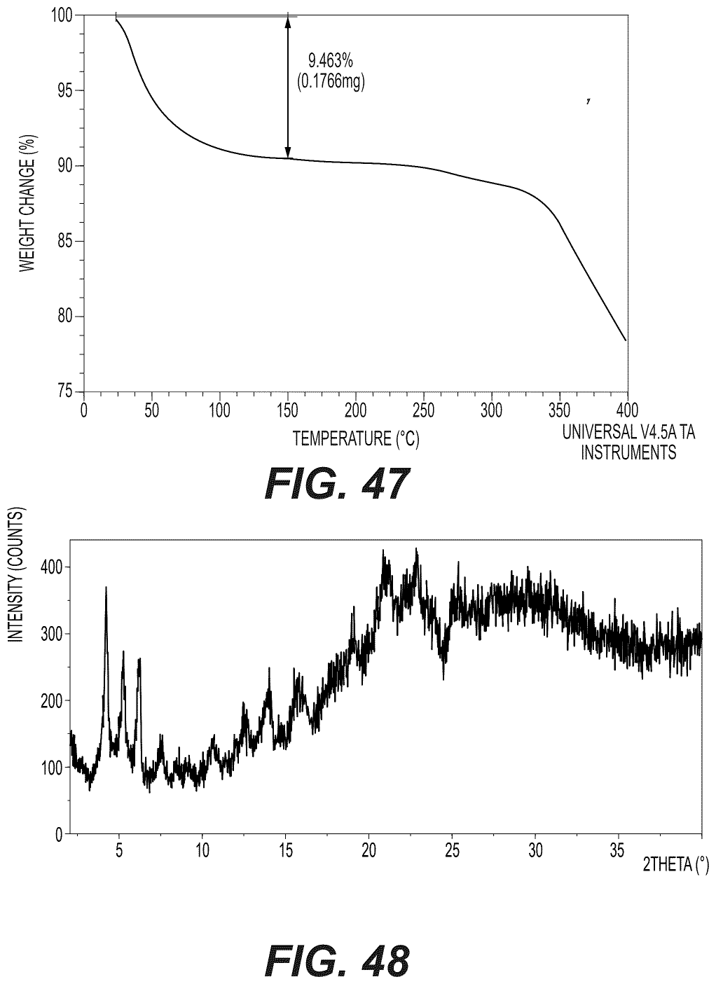

In some embodiments, Formula I, calcium salt, Form II is characterized by a TGA curve substantially as shown in FIG. 47.

In some embodiments, Formula I, calcium salt, Form II has the following properties:
(a) an XRPD pattern substantially as shown in FIG. 45;
(b) a DSC thermogram substantially as shown in FIG. 46; and
(c) a TGA curve substantially as shown in FIG. 47.

In some embodiments, Formula I, calcium salt, Form II is hydrated. In some embodiments, Formula I, calcium salt, Form II is partially or fully hydrated. In some embodiments, Formula I, calcium salt, Form II is fully hydrated.

In some embodiments, the crystalline calcium salt of the compound of Formula I is the Form III (Formula I, calcium salt, Form III). In some embodiments, Formula I, calcium salt, Form III is hydrated. In some embodiments, Formula I, calcium salt, Form III has an XRPD profile substantially as shown in FIG. 48.

In some embodiments, Formula I, calcium salt, Form III has an XRPD pattern comprising degree 2θ-reflections (+/−0.2 degrees 2θ) at 4.2°. 5.2°, and 6.2°. In some embodiments, Formula I, calcium salt, Form III has an XRPD pattern comprising degree 2θ-reflections (+/−0.2 degrees 2θ) at 4.2°, 5.2°, and 6.2°, and one, two or three of the degree 2θ-reflections (+/−0.2 degrees 2θ) at 13.9°, 15.7°, and 21.0°. In some embodiments, Formula I, calcium salt, Form III has an XRPD pattern comprising degree 2θ-reflections (+/−0.2 degrees 2θ) at 4.2°, 5.2°, and 6.2°, and one or two of the degree 2θ-reflections (+/−0.2 degrees 2θ) at 13.9°, 15.7°, and 21.0°. In some embodiments, Formula I, calcium salt, Form III has an XRPD pattern comprising degree 2θ-reflections (+/−0.2 degrees 2θ) at 4.2°, 5.2°, and 6.2°, and one of the degree 2θ-reflections (+/−0.2 degrees 2θ) at 13.9°, 15.7°, and 21.0°. In some embodiments, Formula I, calcium salt, Form III has an XRPD pattern comprising degree 29-reflections (+/−0.2 degrees 2θ) at 4.2°, 5.2°, and 6.2°, and two of the degree 2θ-reflections (+/−0.2 degrees 2θ) at 13.9°, 15.7°, and 21.0°. In some embodiments, Formula I, calcium salt, Form III has an XRPD pattern comprising degree 2θ-reflections (+/−0.2 degrees 2θ) at 4.2°, 5.2°, 6.2°, 13.9°, 15.7°, and 21.0°. In some embodiments, Formula I, calcium salt, Form III has an XRPD pattern comprising any three of the degree 2θ-reflections (+/−0.2 degrees 2θ) at 4.2°, 5.2°, 6.2°, 13.9°, 15.7°, and 21.0°.

In some embodiments, Formula I, calcium salt, Form III has an XRPD pattern comprising degree 2θ-reflections (+/−0.2 degrees 2θ) at 4.2°, 5.2°. 6.2°, 13.9°, 15.7°, and 21.0°, and one, two or three of the degree 2θ-reflections (+/−0.2 degrees 2θ) at 7.5°, 10.6°, and 12.6°. In some embodiments, Formula I, calcium salt, Form III has an XRPD pattern comprising degree 2θ-reflections (+/−0.2 degrees 2θ) at 4.2°. 5.2°, 6.2°, 13.9°, 15.7°, and 21.0°, and one or two of the degree 2θ-reflections (+/−0.2 degrees 2θ) at 7.5°, 10.6°, and 12.6°. In some embodiments, Formula I, calcium salt, Form III has an XRPD pattern comprising degree 2θ-reflections (+/−0.2 degrees 2θ) at 4.2°. 5.2°. 6.2°, 13.9°, 15.7°, and 21.0°, and one of the degree 2θ-reflections (+/−0.2 degrees 2θ) at 7.5°, 10.6°, and 12.6°. In some embodiments, Formula I, calcium salt, Form III has an XRPD pattern comprising degree 2θ-reflections (+/−0.2 degrees 2θ) at 4.2°, 5.2°, 6.2°, 13.9°, 15.7°, and 21.0°, and two of the degree 2θ-reflections (+/−0.2 degrees 2θ) at 7.5°, 10.6°, and 12.6°. In some embodiments, Formula I, calcium salt, Form III has an XRPD pattern comprising degree 2θ-reflections (+/−0.2 degrees 2θ) at 4.2°, 5.2°, 6.2°, 7.5°, 10.6°, 12.6°, 13.9°, 15.7°, and 21.0°. In some embodiments, Formula I, calcium salt, Form III has an XRPD pattern comprising any three of the degree 2θ-reflections (+/−0.2 degrees 2θ) at 4.2°, 5.2°, 6.2°, 7.5°, 10.6°, 12.6°, 13.9°, 15.7°, and 21.0°.

In some embodiments, Formula I, calcium salt, Form III has an XRPD pattern comprising peaks at:

| Pos. | Rel. Int. |
|---|---|
| [°2Th. ± 0.2] | [%] |
| 4.2027 | 100 |
| 5.2494 | 64.58 |
| 6.1747 | 62.82 |
| 7.4813 | 15.12 |
| 10.6013 | 12.69 |
| 12.5542 | 17.46 |
| 13.9203 | 27.71 |
| 15.6797 | 19.8 |
| 21.0148 | 25.12 |

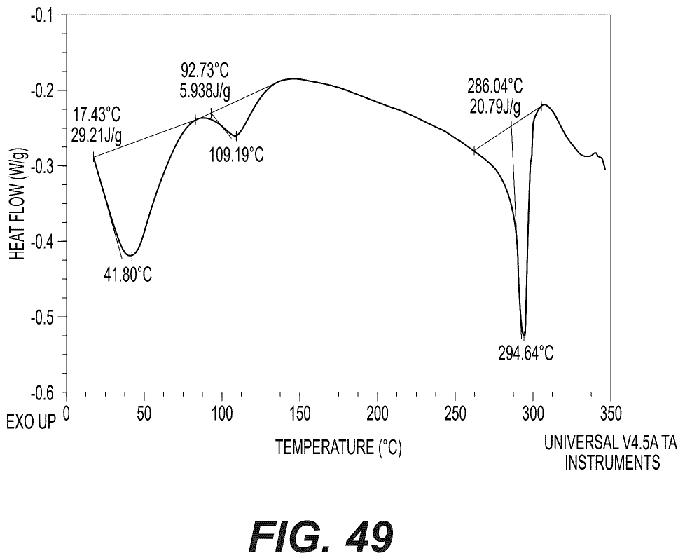

In some embodiments, Formula I, calcium salt, Form III is characterized by a DSC thermogram substantially as shown in FIG. 49.

In some embodiments, Formula I, calcium salt, Form III is characterized by a DSC thermogram having one, two, or three of (i) displays an endothermic transition at about 17° C., (ii) an endothermic transition at about 93° C., and (iii) an endothermic transition at about 286° C.

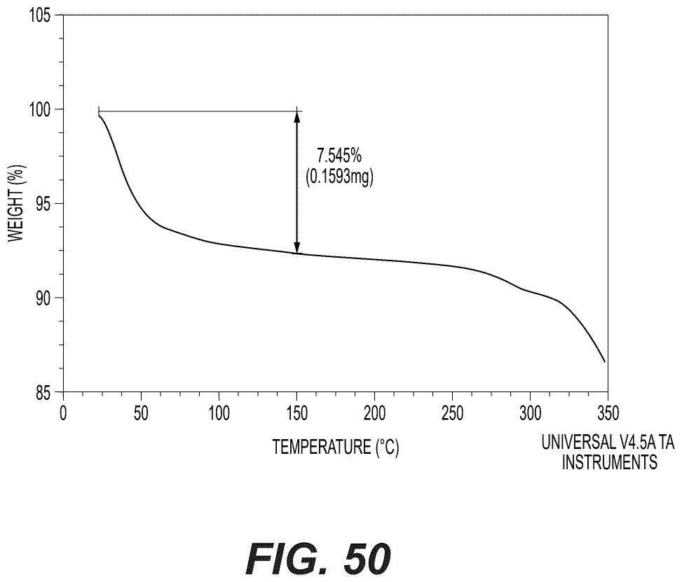

In some embodiments, Formula I, calcium salt, Form III is characterized by a TGA curve substantially as shown in FIG. 50.

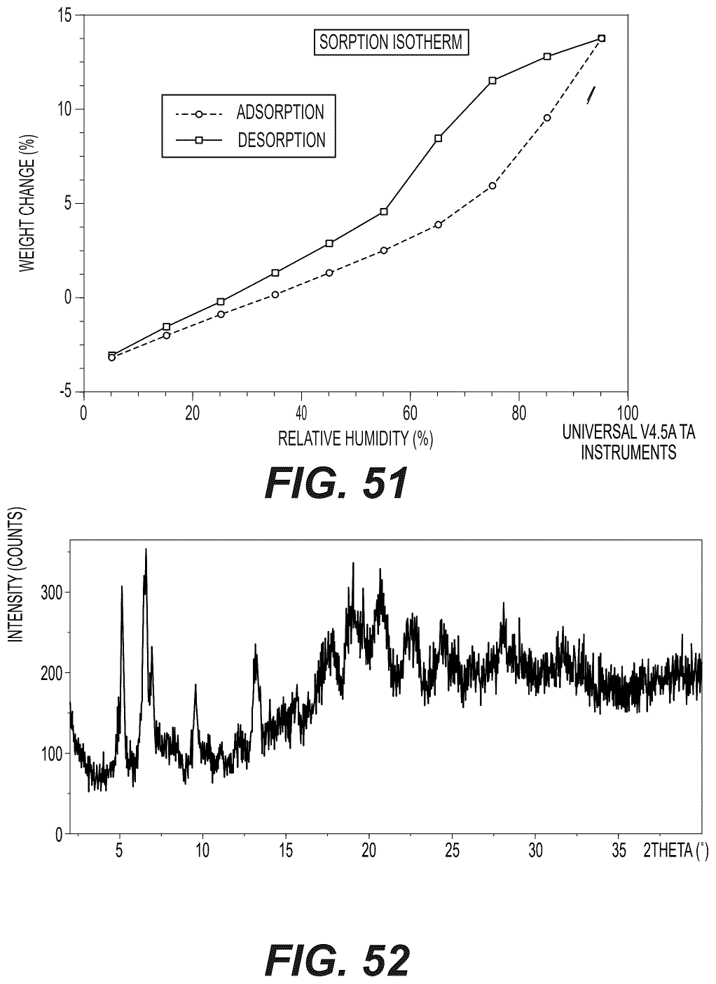

In some embodiments, Formula I, calcium salt, Form III is characterized by a DVS curve substantially as shown in FIG. 51.

In some embodiments, Formula I, calcium salt, Form III has the following properties:
(a) an XRPD pattern substantially as shown in FIG. 48;
(b) a DSC thermogram substantially as shown in FIG. 49;
(c) a TGA curve substantially as shown in FIG. 50; and
(d) a DVS curve substantially as shown in FIG. 51.

In some embodiments, Formula I, calcium salt, Form III is hydrated. In some embodiments, Formula I, calcium salt, Form III is partially or fully hydrated.

In some embodiments, the crystalline calcium salt of the compound of Formula I is the Form IV (Formula I, calcium salt, Form IV). In some embodiments, Formula I, calcium salt, Form IV is hydrated. In some embodiments, Formula I, calcium salt, Form IV has an XRPD profile substantially as shown in FIG. 52.

In some embodiments, Formula I, calcium salt, Form IV has an XRPD pattern comprising degree 2θ-reflections (+/−0.2 degrees 2θ) at 5.2°, 6.5°, and 20.7°. In some embodiments, Formula I, calcium salt, Form IV has an XRPD pattern comprising degree 2θ-reflections (+/−0.2 degrees 2θ) at 5.2°. 6.5°, and 20.7°, and one, two or three of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 2θ) at 5.2°, 6.5°, and 20.7°, and one or two of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 2θ) at 5.2°, 6.5°, and 20.7°, and one of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 20) at 5.2°, 6.5°, and 20.7°, and two of the degree 2θ-reflections (+/−0.2 degrees 2θ) at 7.0°, 13.2°, and 18.9°. In some embodiments, Formula I, calcium salt, Form IV has an XRPD pattern comprising degree 2θ-reflections (+/−0.2 degrees 2θ) at 5.2°, 6.5°, 7.02, 13.2°, 18.9°, and 20.7°. In some embodiments, Formula I, calcium salt, Form IV has an XRPD pattern comprising any three of the degree 2θ-reflections (+/−0.2 degrees 2θ) at 5.2°. 6.5°, 7.0°, 13.2°, 18.9°, and 20.7c.

In some embodiments, Formula I, calcium salt, Form IV has an XRPD pattern comprising degree 2θ-reflections (+/−0.2 degrees 2θ) at 5.2°. 6.5°, 7.0°, 13.2°, 18.9°, and 20.7°, and one, two or three of the degree 2θ-reflections (+/−0.2 degrees 2θ) at 9.6°, 17.8°, and 22.9°. In some embodiments, Formula I, calcium salt, Form IV has an XRPD pattern comprising degree 2θ-reflections (+/−0.2 degrees 2θ) at 5.2°. 6.5°. 7.0°, 13.2°, 18.99, and 20.7, and one or two of the degree 2θ-reflections (+/−0.2 degrees 2θ) at 9.6°, 17.8°, and 22.9°. In some embodiments, Formula I, calcium salt, Form IV has an XRPD pattern comprising degree 2θ-reflections (+/−0.2 degrees 2θ) at 5.2°, 6.5°, 7.0°, 13.2°, 18.9°, and 20.7°, and one of the degree 2θ-reflections (+/−0.2 degrees 2θ) at 9.6°, 17.8°, and 22.9°. In some embodiments. Formula I, calcium salt, Form IV has an XRPD pattern comprising degree 2θ-reflections (+/−0.2 degrees 2θ) at 5.2°, 6.5°, 7.0°, 13.2°, 18.9°, and 20.7°, and two of the degree 2θ-reflections (+/−0.2 degrees 2θ) at 9.6°, 17.8°, and 22.9°. In some embodiments, Formula I, calcium salt, Form IV has an XRPD pattern comprising degree 2θ-reflections (+/−0.2 degrees 2θ) 5.2°, 6.5°, 7.0°, 9.6°, 13.2°, 17.8°, 18.9°, 20.7°, and 22.9°. In some embodiments, Formula I, calcium salt, Form IV has an XRPD pattern comprising any three of the degree 2θ-reflections (+/−0.2 degrees 2θ) at 5.2°, 6.5°, 7.0°, 9.6°, 13.2°, 17.8°, 18.9°, 20.7°, and 22.9°. In some embodiments, Formula I, calcium salt, Form IV has an XRPD pattern comprising at least three of the degree 2θ-reflections (+/−0.2 degrees 2θ) at 5.2°, 6.5°, 7.0°, 8.4°, 9.6°, 13.2°, 14.7°, 17.8°, 18.9°, 20.7°, and 22.9°. In some embodiments, Formula I, calcium salt, Form IV has an XRPD pattern comprising at least four of the degree 2θ-reflections (+/−0.2 degrees 2θ) at 5.2°, 6.5°, 7.0°, 8.4°, 9.6°, 13.2°, 14.7°, 17.8°, 18.9°, 20.7°, and 22.9°. In some embodiments, Formula I, calcium salt, Form IV has an XRPD pattern comprising at least five of the degree 2θ-reflections (+/−0.2 degrees 2θ) at 5.2°, 6.5°, 7.0°, 8.4°, 9.6°, 13.2°, 14.7°, 17.8°, 18.9°, 20.7°, and 22.9°. In some embodiments, Formula I, calcium salt, Form IV has an XRPD pattern comprising peaks at:

| Pos. | Rel. Int. |
|---|---|
| [°2Th. ± 0.2] | [%] |
| 5.1906 | 97.5 |
| 6.5315 | 100 |
| 6.9516 | 63.68 |
| 8.4207 | 11.94 |
| 9.5864 | 38.78 |
| 12.1583 | 7.54 |
| 13.1534 | 44.74 |
| 14.7177 | 14.96 |
| 15.6964 | 20.69 |
| 17.8258 | 43.97 |
| 18.895 | 56.04 |
| 20.7192 | 65.87 |
| 22.8522 | 33.95 |
| 24.3624 | 29.15 |
| 26.1189 | 11.88 |
| 28.138 | 24.29 |

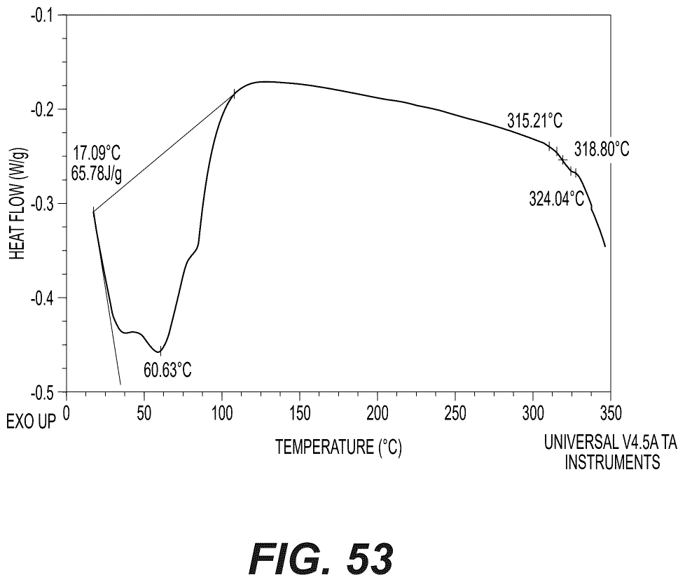

In some embodiments, Formula I, calcium salt, Form IV is characterized by a DSC thermogram substantially as shown in FIG. 53.

In some embodiments, Formula I, calcium salt, Form IV is characterized by a DSC thermogram having one or both of (i) an endothermic transition at about 17° C. and (ii) a baseline shift around 315° C.

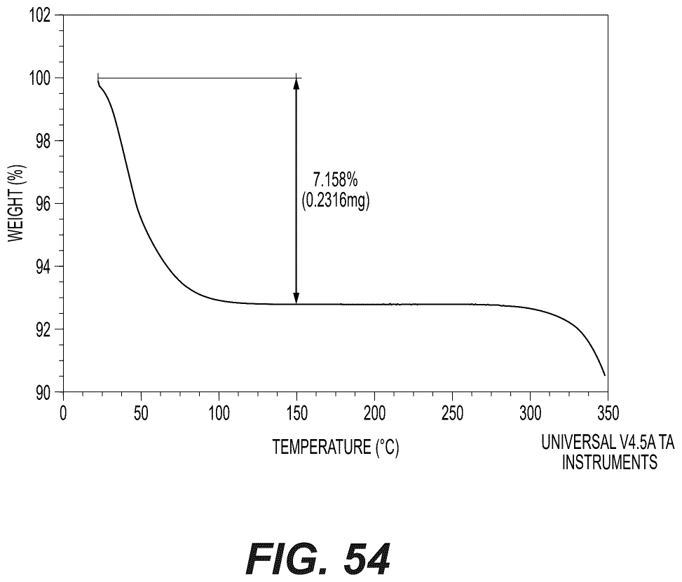

In some embodiments, Formula I, calcium salt, Form IV is characterized by a TGA curve substantially as shown in FIG. 54.

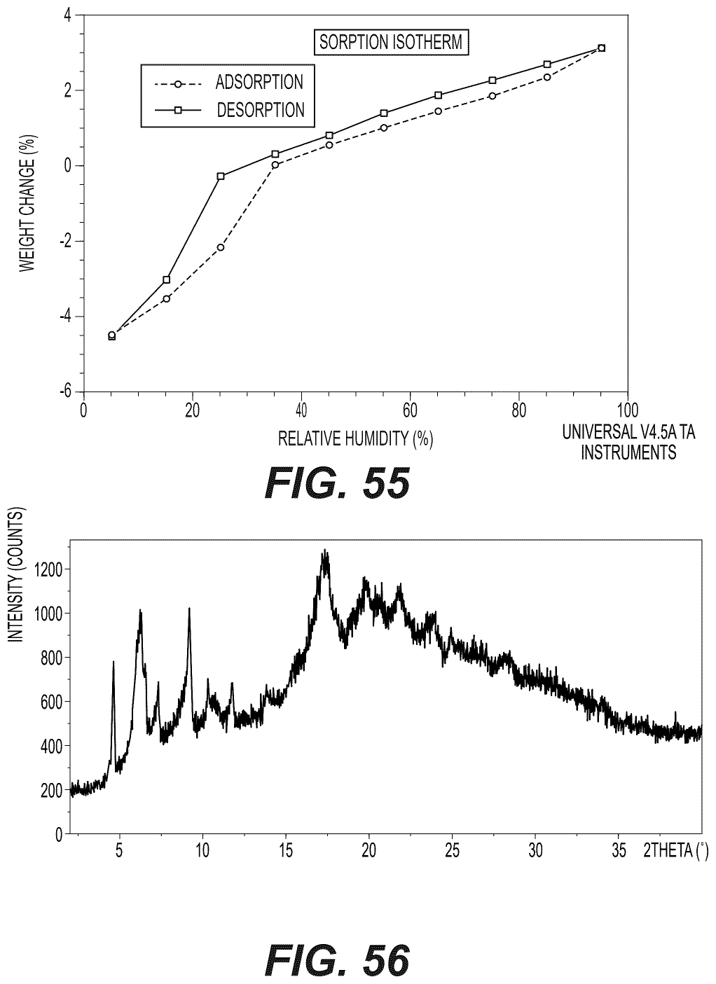

In some embodiments, Formula I, calcium salt, Form IV is characterized by a DVS curve substantially as shown in FIG. 55.

In some embodiments, Formula I, calcium salt, Form IV has the following properties:
(a) an XRPD pattern substantially as shown in FIG. 52;
(b) a DSC thermogram substantially as shown in FIG. 53;
(c) a TGA curve substantially as shown in FIG. 54; and
(d) a DVS curve substantially as shown in FIG. 55.

In some embodiments, Formula I, calcium salt, Form IV is hydrated. In some embodiments, Formula I, calcium salt, Form IV is partially or fully hydrated.

In some embodiments, the crystalline calcium salt of the compound of Formula f is the Form V (Formula I, calcium salt, Form V). In some embodiments, Formula I, calcium salt, Form V is hydrated. In some embodiments, Formula I, calcium salt. Form V has an XRPD profile substantially as shown in FIG. 56.

In some embodiments, Formula I, calcium salt, Form V has an XRPD pattern comprising degree 2θ-reflections (+/−0.2 degrees 2θ) at 4.7°, 6.3°, and 9.2°. In some embodiments, Formula I, calcium salt, Form V has an XRPD pattern comprising degree 2θ-reflections (+/−0.2 degrees 2θ) at 4.7°, 6.3°, and 9.2°, and one, two or three of the degree 2θ-reflections (+/−0.2 degrees 2θ) at 6.6°. 7.3°, and 17.5°. In some embodiments, Formula I, calcium salt, Form V has an XRPD pattern comprising degree 2θ-reflections (+/−0.2 degrees 2θ) at 4.7°, 6.3°, and 9.2°, and one or two of the degree 2θ-reflections (+/−0.2 degrees 2θ) at 6.6°, 7.3°, and 17.5°. In some embodiments, Formula I, calcium salt, Form V has an XRPD pattern comprising degree 2θ-reflections (+/−0.2 degrees 2θ) at 4.7°, 6.3°, and 9.2°, and one of the degree 2θ-reflections (+/−0.2 degrees 2θ) at 6.6°, 7.3°, and 17.5°. In some embodiments, Formula I, calcium salt, Form V has an XRPD pattern comprising degree 2θ-reflections (+/−0.2 degrees 2θ) at 4.7°, 6.3°, and 9.2°, and two of the degree 29-reflections (+/−0.2 degrees 2θ) at 6.6°. 7.3°, and 17.5°. In some embodiments, Formula I, calcium salt, Form V has an XRPD pattern comprising degree 29-reflections (+/−0.2 degrees 2θ) at 4.7°, 6.3°, 6.6°, 7.3°, 9.2°, and 17.5°. In some embodiments, Formula I, calcium salt. Form V has an XRPD pattern comprising any three of the degree 2θ-reflections (+/−0.2 degrees 2θ) at 4.7°, 6.3°, 6.6°, 7.3°, 9.2°, and 17.5°.

In some embodiments, Formula I, calcium salt, Form V has an XRPD pattern comprising degree 2θ-reflections (+/−0.2 degrees 2θ) at 4.7°, 6.3°, 6.6°, 7.3°, 9.2″, and 17.5°, and one, two or three of the degree 2θ-reflections (+/−0.2 degrees 2θ) at 10.3°, 19.8°, and 21.9°. In some embodiments, Formula I, calcium salt, Form V has an XRPD pattern comprising degree 2θ-reflections (+/−0.2 degrees 2θ) at 4.7°, 6.3°, 6.6°, 7.3°, 9.2°, and 17.5°, and one or two of the degree 2θ-reflections (+/−0.2 degrees 2θ) at 10.3°, 19.8°, and 21.9°. In some embodiments, Formula I, calcium salt. Form V has an XRPD pattern comprising degree 2θ-reflections (+/−0.2 degrees 2θ) at 4.7°, 6.3°, 6.6°, 7.3°. 9.2°, and 17.5°, and one of the degree 2θ-reflections (+/−0.2 degrees 2θ) at 10.3°, 19.8°, and 21.9°. In some embodiments, Formula I, calcium salt, Form V has an XRPD pattern comprising degree 2θ-reflections (+/−0.2 degrees 2θ) at 4.7°, 6.3°, 6.6°. 7.3°, 9.22, and 17.5°, and two of the degree 2θ-reflections (+/−0.2 degrees 2θ) at 10.3°, 19.8°, and 21.9°. In some embodiments, Formula I, calcium salt, Form V has an XRPD pattern comprising degree 2θ-reflections (+/−0.2 degrees 2θ) 4.7°. 6.3°, 6.6°, 7.3°. 9.2°, 10.3°, 17.5°, 19.8°, and 21.9°. In some embodiments, Formula I, calcium salt, Form V has an XRPD pattern comprising any three of the degree 2θ-reflections (+/−0.2 degrees 2θ) at 4.7°, 6.3°. 6.6°, 7.3°, 9.2°, 10.3°, 17.5°, 19.8°, and 21.9°.

In some embodiments, Formula I, calcium salt, Form V has an XRPD pattern comprising at least three of the degree 2θ-reflections (+/−0.2 degrees 2θ) at 4.7°, 6.3°, 6.6°, 7.3°, 9.2″, 10.3°, 11.8°, 13.8°, 17.5°, 19.8°, and 21.9°. In some embodiments, Formula I, calcium salt, Form V has an XRPD pattern comprising at least four of the degree 2θ-reflections (+/−0.2 degrees 20) at 4.7°, 6.3°. 6.6°, 7.3°, 9.2°, 10.3°, 11.8°, 13.8°, 17.5°, 19.8°, and 21.9° . . . . In some embodiments, Formula I, calcium salt, Form V has an XRPD pattern comprising at least five of the degree 2θ-reflections (+/−0.2 degrees 2θ) at 4.7°, 6.32, 6.6°, 7.3°, 9.2°, 10.32, 11.8°, 13.8°, 17.5°, 19.8°, and 21.9°.

In some embodiments, Formula I, calcium salt, Form V has an XRPD pattern comprising peaks at:

| Pos. | Rel. Int. |
|---|---|
| [°2Th. ± 0.2] | [%] |
| 4.667286 | 85.49 |
| 6.325596 | 100 |
| 6.574366 | 58.89 |
| 7.337722 | 41.98 |
| 9.219071 | 92.48 |
| 10.33942 | 28.4 |
| 11.76972 | 25.41 |
| 13.8499 | 17.35 |
| 17.51082 | 71.54 |
| 19.82631 | 37.1 |
| 21.93564 | 26.21 |
| 23.9084 | 19.19 |
| 28.46511 | 11.11 |

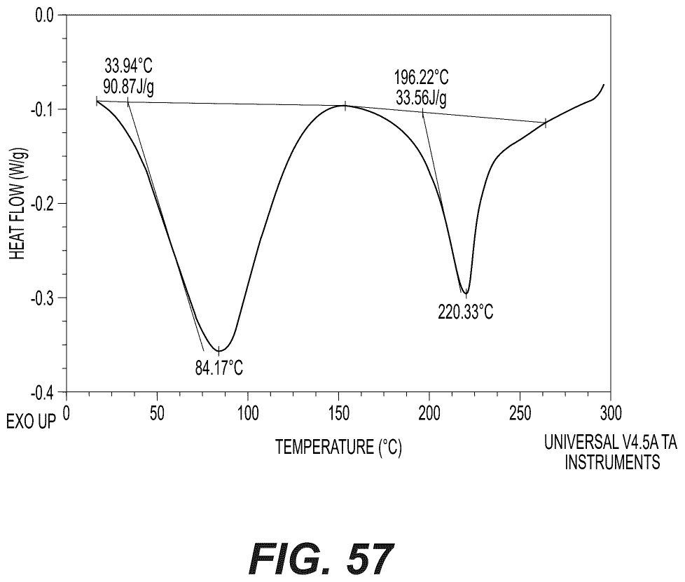

In some embodiments, Formula I, calcium salt, Form V is characterized by a DSC thermogram substantially as shown in FIG. 57.

In some embodiments, Formula I, calcium salt, Form V is characterized by a DSC thermogram having one or both of (i) an endothermic transition at about 34° C. and (ii) an endothermic transition around 196° C.

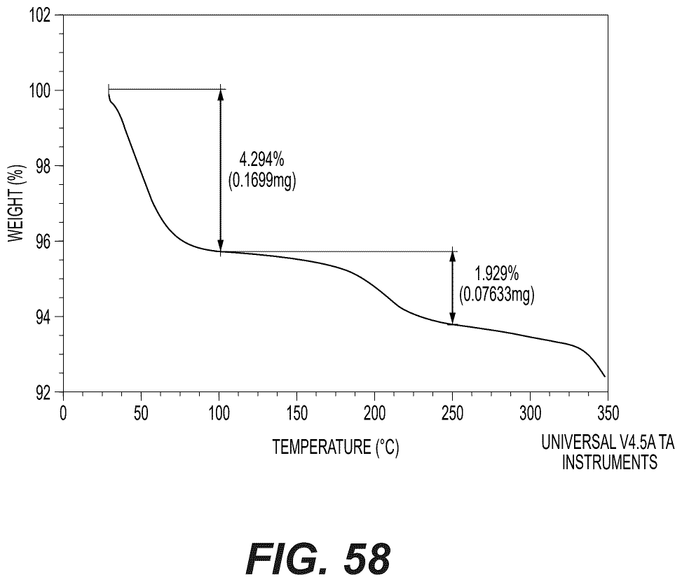

In some embodiments, Formula I, calcium salt, Form V is characterized by a TGA curve substantially as shown in FIG. 58.

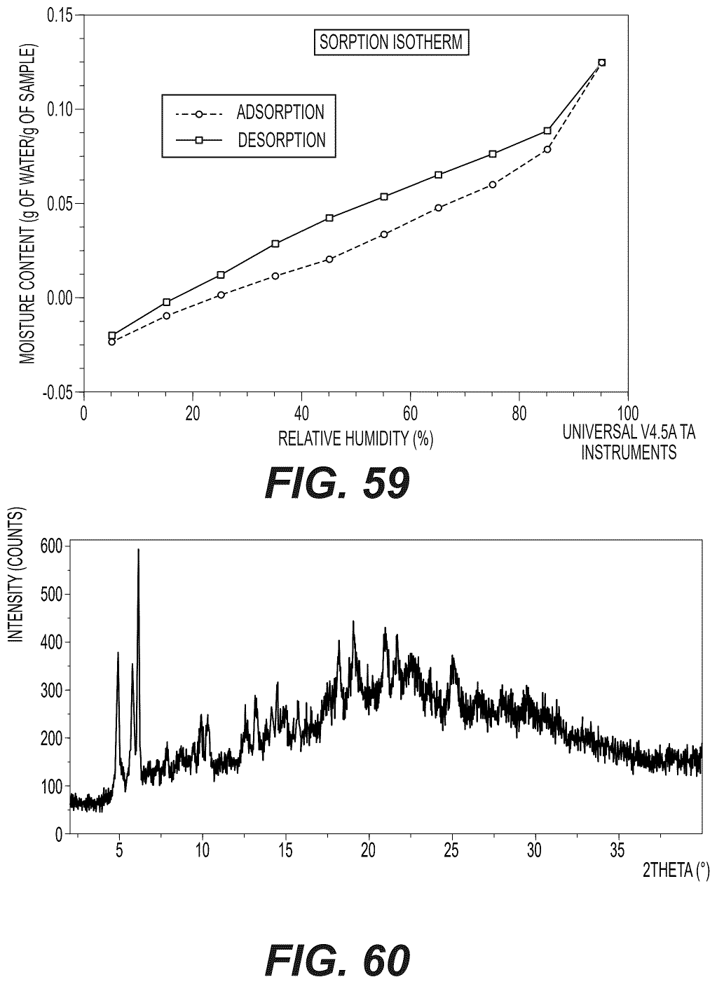

In some embodiments, Formula I, calcium salt, Form V is characterized by a DVS curve substantially as shown in FIG. 59.

In some embodiments, Formula I, calcium salt, Form V has the following properties:
(a) an XRPD pattern substantially as shown in FIG. 56;
(b) a DSC thermogram substantially as shown in FIG. 57;
(c) a TGA curve substantially as shown in FIG. 58; and
(d) a DVS curve substantially as shown in FIG. 59.

In some embodiments, Formula I, calcium salt, Form V is hydrated. In some embodiments, Formula I, calcium salt, Form V is partially or fully hydrated.

In some embodiments, the crystalline calcium salt of the compound of Formula I is the Form VI (Formula I, calcium salt, Form VI). In some embodiments, Formula I, calcium salt, Form VI is hydrated. In some embodiments, Formula I, calcium salt. Form VI has an XRPD profile substantially as shown in FIG. 60.

In some embodiments, Formula I, calcium salt, Form VI has an XRPD pattern comprising degree 2θ-reflections (+/−0.2 degrees 2θ) at 5.0°. 5.8°, and 6.2°. In some embodiments, Formula I, calcium salt, Form VI has an XRPD pattern comprising degree 2θ-reflections (+/−0.2 degrees 2θ) at 5.0°, 5.8°, and 6.2°, and one, two or three of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, and 6.2°, and one or two of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, and 6.2°, and one of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, and 6.2°, and two of the degree 2θ-reflections (+/−0.2 degrees 2θ) at 18.2°, 19.1°, and 21.0°. In some embodiments, Formula I, calcium salt, Form VI has an XRPD pattern comprising degree 2θ-reflections (+/−0.2 degrees 2θ) at 5.0°, 5.8°, 6.2°, 18.2°, 19.1°, and 21.0°. In some embodiments, Formula I, calcium salt, Form VI has an XRPD pattern comprising any three of the degree 2θ-reflections (+/−0.2 degrees 2θ) at 5.0°, 5.8°. 6.2°, 18.2°, 19.1°, and 21.0°.

In some embodiments, Formula I, calcium salt, Form VI has an XRPD pattern comprising degree 2θ-reflections (+/−0.2 degrees 2θ) at 5.0°, 5.8°. 6.2°, 18.2°, 19.1°, and 21.0°, and one, two or three of the degree 2θ-reflections (+/−0.2 degrees 2θ) at 10.0°, 10.3°, and 21.7°. In some embodiments, Formula I, calcium salt, Form VI has an XRPD pattern comprising degree 2θ-reflections (+/−0.2 degrees 2θ) at 5.0°, 5.8°, 6.2°, 18.22, 19.1°, and 21.0°, and one or two of the degree 2θ-reflections (+/−0.2 degrees 2θ) at 10.0°, 10.3°, and 21.7°. In some embodiments, Formula I, calcium salt, Form VI has an XRPD pattern comprising degree 2θ-reflections (+/−0.2 degrees 2θ) at 5.0°. 5.8°. 6.2°, 18.2°, 19.1°, and 21.0°, and one of the degree 2θ-reflections (+/−0.2 degrees 2θ) at 10.0°, 10.3°, and 21.7°. In some embodiments, Formula I, calcium salt, Form VI has an XRPD pattern comprising degree 2θ-reflections (+/−0.2 degrees 2θ) at 5.0°, 5.8°, 6.2°, 18.2°, 19.1°, and 21.0°, and two of the degree 2θ-reflections (+/−0.2 degrees 2θ) at 10.0°, 10.3°, and 21.7°. In some embodiments, Formula I, calcium salt, Form VI has an XRPD pattern comprising degree 2θ-reflections (+/−0.2 degrees 2θ) 5.0°, 5.8°, 6.2°, 10.0°, 10.3°, 18.2°, 19.1°, 21.0°, and 21.7°. In some embodiments, Formula I, calcium salt, Form VI has an XRPD pattern comprising any three of the degree 2θ-reflections (+/−0.2 degrees 2θ) at 5.0°, 5.8°, 6.2°, 10.0°, 10.3°, 18.2°, 19.1°, 21.0°, and 21.7°.

In some embodiments, Formula I, calcium salt, Form VI has an XRPD pattern comprising at least three of the degree 2θ-reflections (+/−0.2 degrees 2θ) at 5.0°, 5.8°, 6.2°, 7.9°, 10.0°, 10.3°, 12.6°, 13.2°, 14.5°, 15.8°, 18.2°, 19.1°, 21.0°, and 21.7°. In some embodiments, Formula I, calcium salt, Form VI has an XRPD pattern comprising at least four of the degree 2θ-reflections (+/−0.2 degrees 2θ) at 5.0°, 5.8°, 6.2°, 7.9°, 10.0°, 10.3°, 12.6°, 13.2°, 14.5°, 15.8°, 18.2°, 19.1°, 21.0°, and 21.7°. In some embodiments, Formula I, calcium salt, Form VI has an XRPD pattern comprising at least five of the degree 2θ-reflections (+/−0.2 degrees 2θ) at 5.0°, 5.8°, 6.2°, 7.9°, 10.0°, 10.3°, 12.6°, 13.2°, 14.5°, 15.8°, 18.2°, 19.1°, 21.0°, and 21.7°.

In some embodiments, Formula I, calcium salt, Form VI has an XRPD pattern comprising peaks at:

| Pos. | Rel. Int. |
|---|---|
| [°2Th. ± 0.2] | [%] |
| 4.97 | 63.7 |
| 5.83 | 53.94 |
| 6.21 | 100 |
| 7.90 | 10.79 |
| 8.65 | 6.8 |
| 9.95 | 22.63 |
| 10.34 | 21.71 |
| 12.61 | 15.78 |
| 13.22 | 19.38 |
| 14.50 | 20.38 |
| 15.77 | 12.66 |
| 18.22 | 29.81 |
| 19.11 | 35.21 |
| 20.98 | 27.28 |
| 21.71 | 26.79 |
| 22.61 | 16.35 |
| 24.99 | 20.77 |
| 26.59 | 8.56 |
| 29.60 | 9.96 |

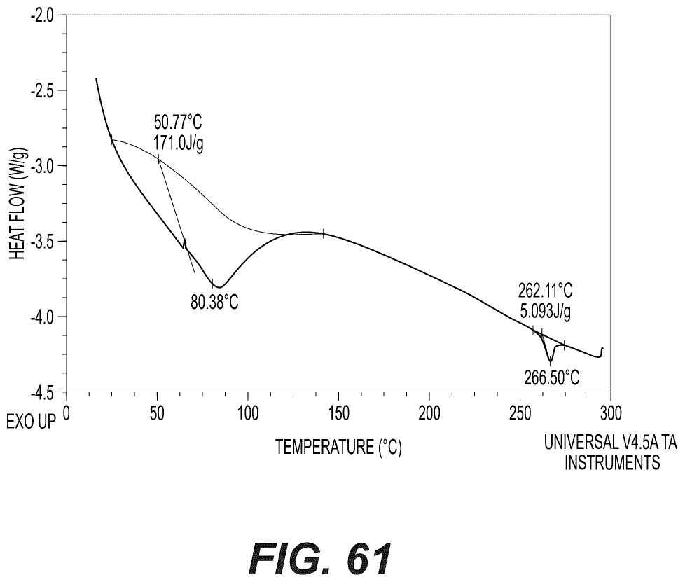

In some embodiments, Formula I, calcium salt, Form VI is characterized by a DSC thermogram substantially as shown in FIG. 61.

In some embodiments, Formula I, calcium salt, Form VI is characterized by a DSC thermogram having one or both of (i) an endothermic transition at about 51° C. and (ii) an endothermic transition around 262° C.

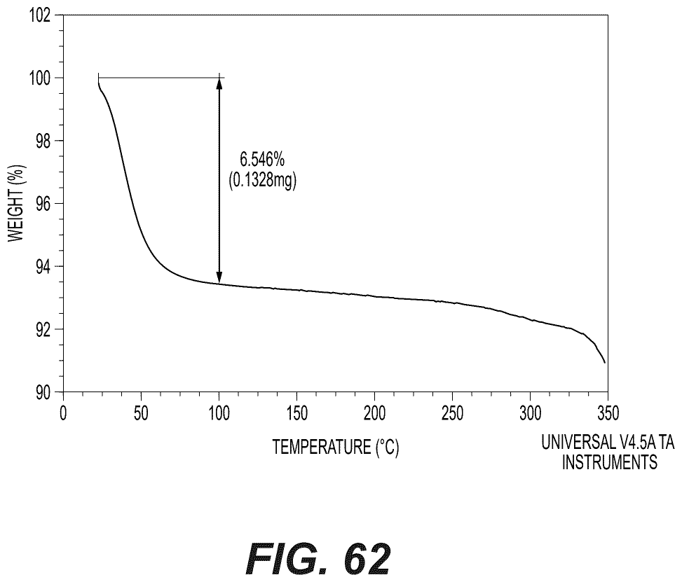

In some embodiments, Formula I, calcium salt, Form VI is characterized by a TGA curve substantially as shown in FIG. 62.

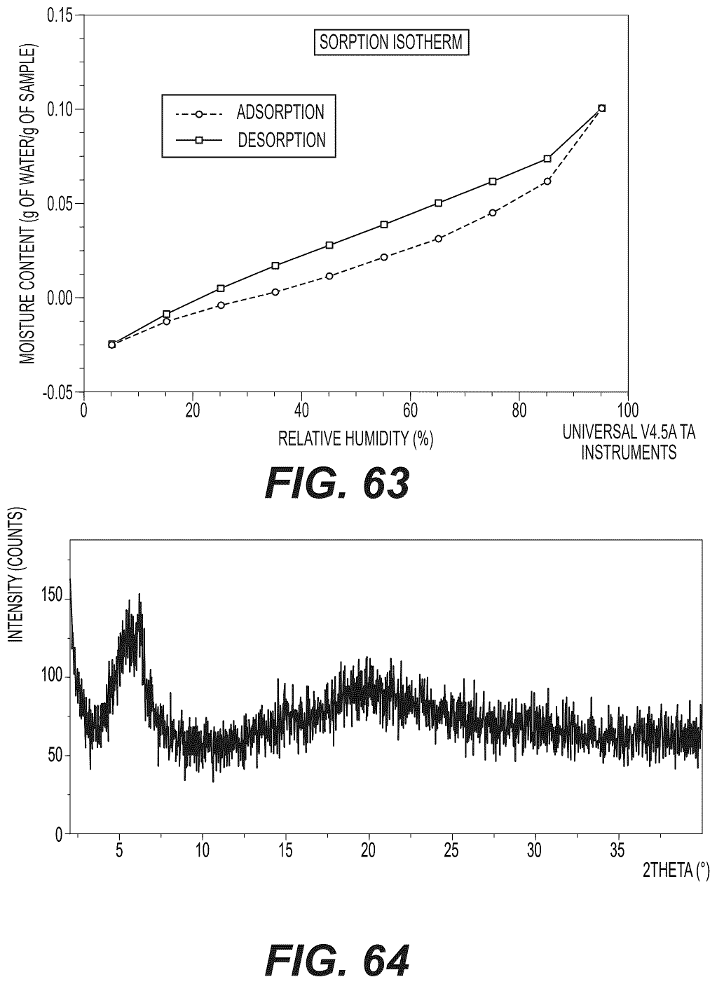

In some embodiments, Formula I, calcium salt, Form VI is characterized by a DVS curve substantially as shown in FIG. 63.

In some embodiments, Formula I, calcium salt, Form VI has the following properties:
(a) an XRPD pattern substantially as shown in FIG. 60;
(b) a DSC thermogram substantially as shown in FIG. 61;
(c) a TGA curve substantially as shown in FIG. 62; and
(d) a DVS curve substantially as shown in FIG. 63.

In some embodiments, Formula I, calcium salt, Form VI is hydrated. In some embodiments, Formula I, calcium salt, Form VI is partially or fully hydrated.

In some embodiments, the calcium salt of the compound of Formula I is amorphous (Formula I, calcium salt, amorphous). In some embodiments, Formula I, calcium salt, amorphous has an XRPD profile substantially as shown in FIG. 64.

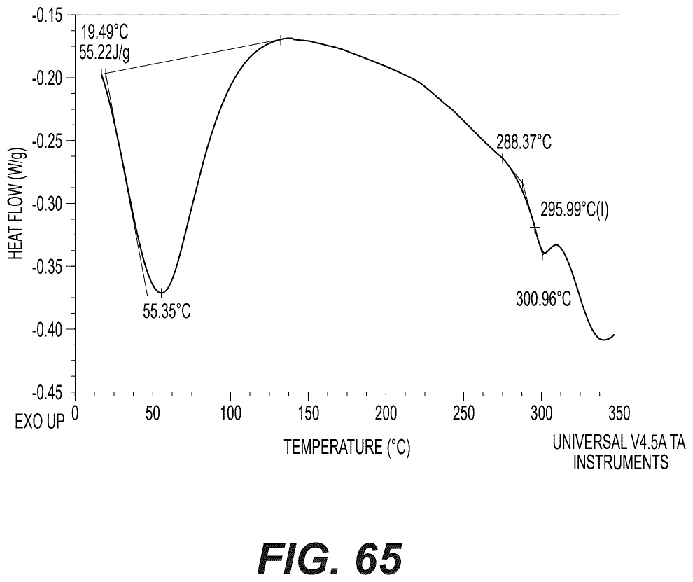

In some embodiments, Formula I, calcium salt, amorphous is characterized by a DSC thermogram substantially as shown in FIG. 65.

In some embodiments, Formula I, calcium salt, amorphous is characterized by a DSC thermogram having a baseline shift around 288° C.

### Compound of Formula I, Diethylamine Salt

In some embodiments, the compound of Formula I can be isolated as a diethylamine salt which can be amorphous or crystalline. In some embodiments, the diethylamine salt of the compound of Formula I is crystalline (crystalline diethylamine salt of the compound of Formula I). In some embodiments, the crystalline diethylamine salt of the compound of Formula Lis unsolvated.

In some embodiments, the crystalline diethylamine salt of the compound of Formula I has an XRPD profile substantially as shown in FIG. 29.

In some embodiments, the crystalline diethylamine salt of the compound of Formula I has an XRPD pattern comprising degree 2θ-reflections (+/−0.2 degrees 2θ) at 10.7°, 18.5°, and 19.3°. In some embodiments, the crystalline diethylamine salt of the compound of Formula I has an XRPD pattern comprising degree 2θ-reflections (+/−0.2 degrees 2θ) at 10.7°, 18.5°, and 19.3°, and one, two or three of the degree 2θ-reflections (+/−0.2 degrees 2θ) at 9.3°, 20.4°, and 21.5°. In some embodiments, the crystalline diethylamine salt of the compound of Formula I has an XRPD pattern comprising degree 2θ-reflections (+/−0.2 degrees 2θ) at 10.7°, 18.5°, and 19.3°, and one or two of the degree 2θ-reflections (+/−0.2 degrees 2θ) at 9.39, 20.4°, and 21.59. In some embodiments, the crystalline diethylamine salt of the compound of Formula I has an XRPD pattern comprising degree 2θ-reflections (+/−0.2 degrees 2θ) at 10.7°, 18.5°, and 19.3°, and one of the degree 2θ-reflections (+/−0.2 degrees 2θ) at 9.3°, 20.4°, and 21.5°. In some embodiments, the crystalline diethylamine salt of the compound of Formula I has an XRPD pattern comprising degree 2θ-reflections (+/−0.2 degrees 2θ) at 10.7°, 18.5°, and 19.3°, and two of the degree 2θ-reflections (+/−0.2 degrees 2θ) at 9.3°, 20.49, and 21.5°. In some embodiments, the crystalline diethylamine salt of the compound of Formula I has an XRPD pattern comprising degree 2θ-reflections (+/−0.2 degrees 2θ) at 9.3°, 10.7°, 18.5°, 19.3°, 20.4°, and 21.5°. In some embodiments, the crystalline diethylamine salt of the compound of Formula I has an XRPD pattern comprising any three of the degree 2θ-reflections (+/−0.2 degrees 2θ) at 9.3°, 10.7°, 18.5°, 19.3°, 20.4°, and 21.5°.

In some embodiments, the crystalline diethylamine salt of the compound of Formula I has an XRPD pattern comprising degree 2θ-reflections (+/−0.2 degrees 2θ) at 9.3°, 10.7°, 18.5°. 19.3°, 20.4°, and 21.5°, and one, two or three of the degree 2θ-reflections (+/−0.2 degrees 2θ) at 9.1°, 11.8°, and 17.0°. In some embodiments, the crystalline diethylamine salt of the compound of Formula I has an XRPD pattern comprising degree 2θ-reflections (+/−0.2 degrees 2θ) at 9.3°, 10.72, 18.5°, 19.3°, 20.4°, and 21.5°, and one or two of the degree 2θ-reflections (+/−0.2 degrees 2θ) at 9.1°, 11.8°, and 17.0°. In some embodiments, the crystalline diethylamine salt of the compound of Formula I has an XRPD pattern comprising degree 2θ-reflections (+/−0.2 degrees 2θ) at 9.3°, 10.7°, 18.5°, 19.3°, 20.4°, and 21.5°, and one of the degree 2θ-reflections (+/−0.2 degrees 2θ) at 9.1°, 11.8°, and 17.0°. In some embodiments, the crystalline diethylamine salt of the compound of Formula I has an XRPD pattern comprising degree 2θ-reflections (+/−0.2 degrees 2θ) at 9.3°, 10.7°, 18.5°, 19.3°, 20.4°, and 21.5°, and two of the degree 2θ-reflections (+/−0.2 degrees 2θ) at 9.1°, 11.8°, and 17.0°. In some embodiments, the crystalline diethylamine salt of the compound of Formula I has an XRPD pattern comprising degree 2θ-reflections (+/−0.2 degrees 2θ) at 9.1°, 9.3°, 10.7°, 11.8°, 17.0°, 18.5°, 19.3°, 20.4°, and 21.5°. In some embodiments, the crystalline diethylamine salt of the compound of Formula I has an XRPD pattern comprising any three of the degree 2θ-reflections (+/−0.2 degrees 2θ) at 9.1°. 9.3°, 10.7°, 11.8°, 17.0°, 18.5°, 19.3°, 20.4°, and 21.5°. In some embodiments, the crystalline diethylamine salt of the compound of Formula I has an XRPD pattern comprising at least three of the degree 2θ-reflections (+/−0.2 degrees 2θ) at 9.1°, 9.3°, 10.7°, 11.8°, 13.5°, 16.0°, 17.0°, 18.5°, 19.3°, 20.4°, and 21.5°. In some embodiments, the crystalline diethylamine salt of the compound of Formula I has an XRPD pattern comprising at least four of the degree 2θ-reflections (+/−0.2 degrees 2θ) at 9.1°, 9.3°, 10.7°, 11.8°, 13.5°, 16.0°, 17.0°, 18.5°, 19.3°, 20.4°, and 21.5°. In some embodiments, the crystalline diethylamine salt of the compound of Formula I has an XRPD pattern comprising at least five of the degree 2θ-reflections (+/−0.2 degrees 2θ) at 9.1°, 9.3°, 10.7°, 11.8°, 13.5°, 16.0°, 17.0°, 18.5°, 19.3°, 20.4°, and 21.5°. In some embodiments, the crystalline diethylamine salt of the compound of Formula I has an XRPD pattern comprising peaks at:

| Pos. | Rel. Int. |
|---|---|
| [°2Th. ± 0.2] | [%] |
| 9.0600 | 33.02 |
| 9.2858 | 59.35 |
| 10.6772 | 89.06 |
| 11.7972 | 43.10 |
| 13.5390 | 24.66 |
| 16.0415 | 28.61 |
| 17.0096 | 41.16 |
| 18.4981 | 68.99 |
| 19.3448 | 100.00 |
| 20.4488 | 66.46 |
| 21.4765 | 64.81 |
| 23.3941 | 24.79 |
| 25.8753 | 23.86 |
| 26.9302 | 13.33 |
| 29.9768 | 13.89 |
| 31.8091 | 13.95 |

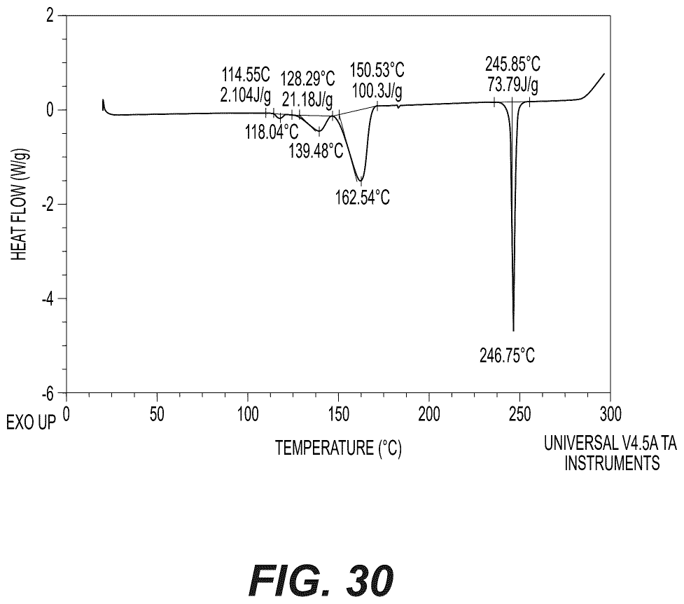

In some embodiments, the crystalline diethylamine salt of the compound of Formula I is characterized by a DSC thermogram substantially as shown in FIG. 30.

In some embodiments, the crystalline diethylamine salt of the compound of Formula I is characterized by a DSC thermogram having one, two, three, or all four of (i) an endothermic transition at about 115° C., (ii) an endothermic transition at about 128° C., (iii) an endothermic transition at about 151° C., and (iv) and an endothermic transition at about 246° C.

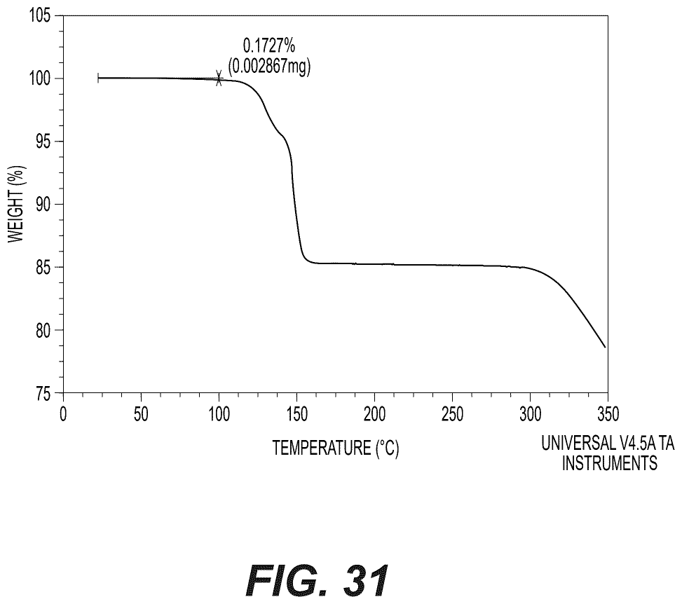

In some embodiments, the crystalline diethylamine salt of the compound of Formula Tis characterized by a TOA curve substantially as shown in FIG. 31.

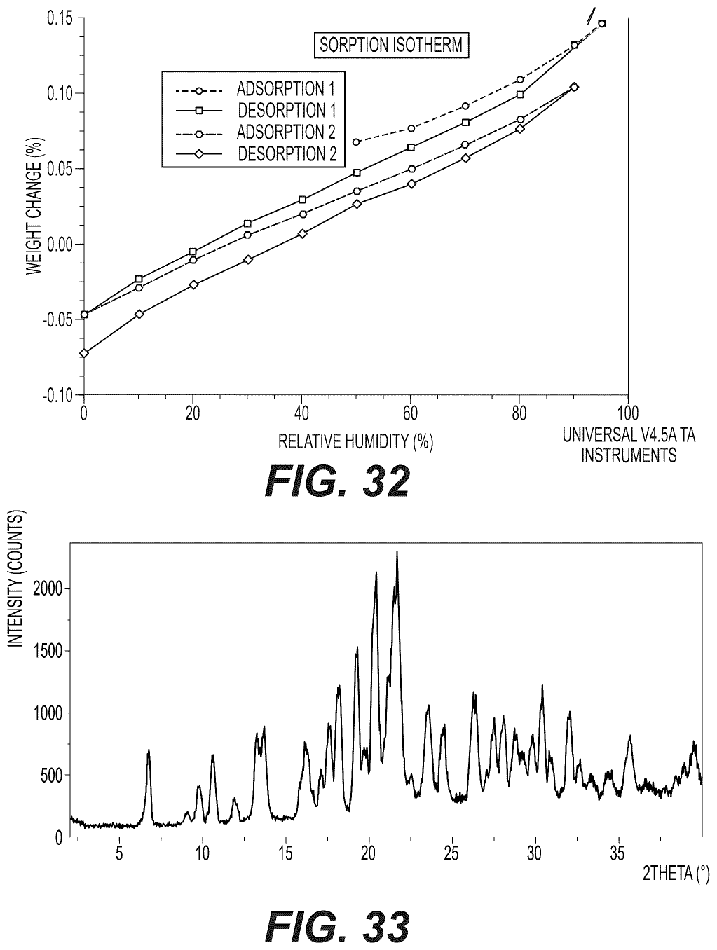

In some embodiments, the crystalline diethylamine salt of the compound of Formula I is characterized by a DVS curve substantially as shown in shown in FIG. 32.

In some embodiments, the crystalline diethylamine salt of the compound of Formula I has the following properties:
(a) an XRPD pattern substantially as shown in FIG. 29;
(b) a DSC thermogram substantially as shown in FIG. 30;
(c) a TGA curve substantially as shown in FIG. 31; and
(d) a DVS curve substantially as shown in FIG. 32.

In some embodiments, the crystalline diethylamine salt of the compound of Formula I is anhydrous. In some embodiments, the crystalline diethylamine salt of the compound of Formula I is unsolvated.

### Compound of Formula I, Diethanolamine Salt

In some embodiments, the compound of Formula I can be isolated as a diethanolamine salt which can be amorphous or crystalline. In some embodiments, the diethanolamine salt of the compound of Formula I is crystalline (crystalline diethanolamine salt of the compound of Formula I). In some embodiments, the crystalline diethanolamine salt of the compound of Formula I is unsolvated.

In some embodiments, the crystalline diethanolamine salt of the compound of Formula I has an XRPD profile substantially as shown in FIG. 33.

In some embodiments, the crystalline diethanolamine salt of the compound of Formula I has an XRPD pattern comprising degree 2θ-reflections (+/−0.2 degrees 2θ) at 19.3°, 20.4°, 21.7°. In some embodiments, the crystalline diethanolamine salt of the compound of Formula I has an XRPD pattern comprising degree 2θ-reflections (+/−0.2 degrees 2θ) at 19.3°, 20.4°. 21.7°, and one, two or three of the degree 2θ-reflections (+/−0.2 degrees 2θ) at 18.2°, 26.2°, and 30.4°. In some embodiments, the crystalline diethanolamine salt of the compound of Formula I has an XRPD pattern comprising degree 2θ-reflections (+/−0.2 degrees 2θ) at 19.3°, 20.4°, 21.7°, and one or two of the degree 2θ-reflections (+/−0.2 degrees 2θ) at 18.2°, 26.2°, and 30.4°. In some embodiments, the crystalline diethanolamine salt of the compound of Formula I has an XRPD pattern comprising degree 2θ-reflections (+/−0.2 degrees 2θ) at 19.3°, 20.4°, 21.7°, and one of the degree 2θ-reflections (+/−0.2 degrees 2θ) at 18.2°, 26.2°, and 30.4°. In some embodiments, the crystalline diethanolamine salt of the compound of Formula I has an XRPD pattern comprising degree 2θ-reflections (+/−0.2 degrees 2θ) at 19.3°, 20.4°, 21.7°, and two of the degree 2θ-reflections (+/−0.2 degrees 2θ) at 18.2°, 26.2°, and 30.4°. In some embodiments, the crystalline diethanolamine salt of the compound of Formula I has an XRPD pattern comprising degree 2θ-reflections (+/−0.2 degrees 2θ) at 18.2°, 19.3°, 20.4°, 21.7°, 26.2°, and 30.4°. In some embodiments, the crystalline diethanolamine salt of the compound of Formula I has an XRPD pattern comprising any three of the degree 2θ-reflections (+/−0.2 degrees 2θ) at 18.2°, 19.3°, 20.4°, 21.7°, 26.2°, and 30.4°.

In some embodiments, the crystalline diethanolamine salt of the compound of Formula I has an XRPD pattern comprising degree 2θ-reflections (+/−0.2 degrees 2θ) at 18.2°, 19.3°. 20.4°, 21.7°, 26.2°, and 30.4°, and one, two or three of the degree 2θ-reflections (+/−0.2 degrees 2θ) at 6.7°, 13.7°, and 23.5°. In some embodiments, the crystalline diethanolamine salt of the compound of Formula I has an XRPD pattern comprising degree 2θ-reflections (+/−0.2 degrees 2θ) at 18.2°, 19.3°, 20.4°, 21.7°, 26.2°, and 30.4°, and one or two of the degree 2θ-reflections (+/−0.2 degrees 2θ) at 6.70, 13.7°, and 23.5°. In some embodiments, the crystalline diethanolamine salt of the compound of Formula I has an XRPD pattern comprising degree 29-reflections (+/−0.2 degrees 2θ) at 18.2°, 19.3°, 20.4°, 21.7°, 26.2°, and 30.4°, and one of the degree 2θ-reflections (+/−0.2 degrees 2θ) at 6.7°, 13.7°, and 23.5°. In some embodiments, the crystalline diethanolamine salt of the compound of Formula I has an XRPD pattern comprising degree 2θ-reflections (+/−0.2 degrees 2θ) at 18.2°, 19.3°, 20.4°, 21.7°, 26.2°, and 30.4°, and two of the degree 2θ-reflections (+/−0.2 degrees 2θ) at 6.7°, 13.7°, and 23.5°. In some embodiments, the crystalline diethanolamine salt of the compound of Formula I has an XRPD pattern comprising degree 2θ-reflections (+/−0.2 degrees 2θ) at 6.7°, 13.7°, 18.2°, 19.3°, 20.4°, 21.7°, 23.5°, 26.2°, and 30.4°. In some embodiments, the crystalline diethanolamine salt of the compound of Formula I has an XRPD pattern comprising any three of the degree 2θ-reflections (+/−0.2 degrees 2θ) at 6.7°, 13.7°, 18.2°, 19.3°, 20.4°, 21.7°, 23.5°, 26.2°, and 30.4°. In some embodiments, the crystalline diethanolamine salt of the compound of Formula I has an XRPD pattern comprising at least three of the degree 2θ-reflections (+/−0.2 degrees 2θ) at 6.7°. 9.7°, 10.6°, 13.7°, 16.2°, 17.5°, 18.2°, 19.3°, 20.4°, 21.7°, 23.5°, 24.4°, 26.2°, 28.0°, 30.4°, and 32.0°. In some embodiments, the crystalline diethanolamine salt of the compound of Formula I has an XRPD pattern comprising at least four of the degree 2θ-reflections (+/−0.2 degrees 2θ) at 6.7°, 9.7°, 10.6°, 13.7°, 16.2°, 17.5°, 18.2°, 19.3°, 20.4°, 21.7°, 23.5°, 24.4°, 26.2°, 28.0°, 30.4°, and 32.0°. In some embodiments, the crystalline diethanolamine salt of the compound of Formula I has an XRPD pattern comprising at least five of the degree 2θ-reflections (+/−0.2 degrees 2θ) at 6.7°, 9.7°, 10.6°, 13.7°, 16.2°, 17.5°, 18.2°, 19.3°, 20.4°, 21.7°, 23.5°, 24.4°, 26.2°, 28.0°, 30.4°, and 32.0°.

In some embodiments, the crystalline diethanolamine salt of the compound of Formula I has an XRPD pattern comprising peaks at:

| Pos. | Rel. Int. |
|---|---|
| [°2Th. ± 0.2] | [%] |
| 6.7260 | 35.01 |
| 9.6955 | 15.62 |
| 10.5501 | 29.19 |
| 13.7067 | 41.39 |
| 16.2291 | 28.31 |
| 17.5184 | 33.92 |
| 18.1604 | 54.48 |
| 19.2910 | 68.26 |
| 20.3546 | 93.10 |
| 21.6826 | 100.00 |
| 23.5064 | 39.27 |
| 24.4165 | 31.06 |
| 26.1929 | 41.71 |
| 28.0623 | 30.70 |
| 30.3657 | 42.58 |
| 31.9905 | 36.70 |
| 35.6863 | 23.91 |

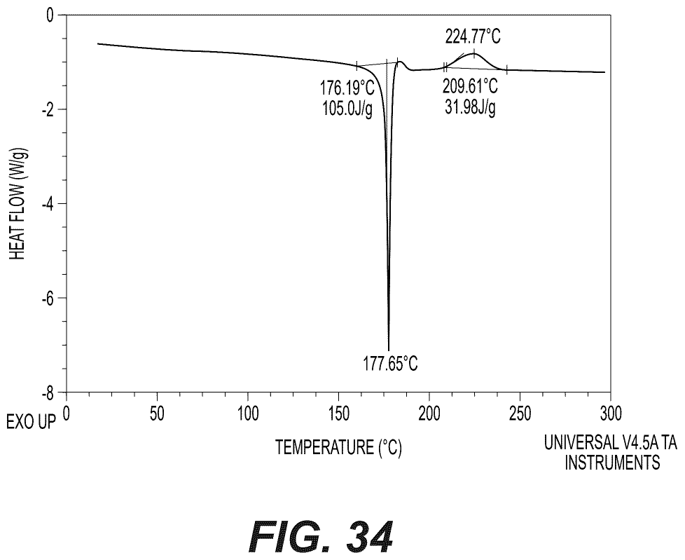

In some embodiments, the crystalline diethanolamine salt of the compound of Formula I is characterized by a DSC thermogram substantially as shown in FIG. 34.

In some embodiments, the crystalline diethanolamine salt of the compound of Formula I is characterized by a DSC thermogram having one or both of (i) an endothermic transition with an onset at about 176° C. and (ii) an exothermic transition at about 210° C.

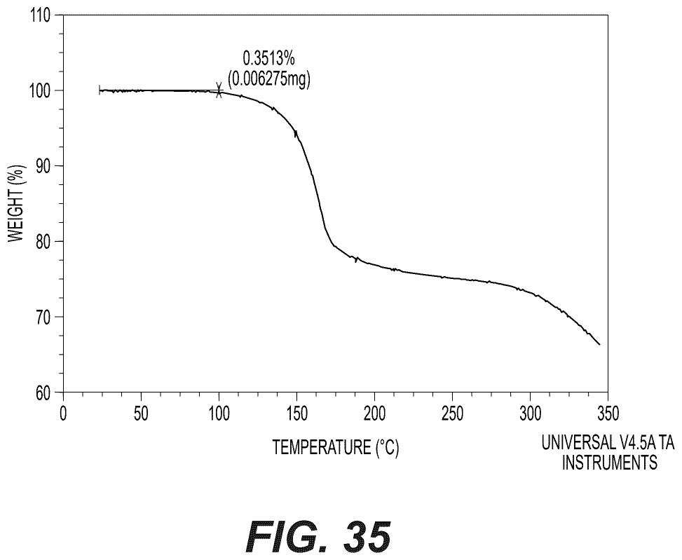

In some embodiments, the crystalline diethanolamine salt of the compound of Formula I is characterized by a TGA curve substantially as shown in FIG. 35.

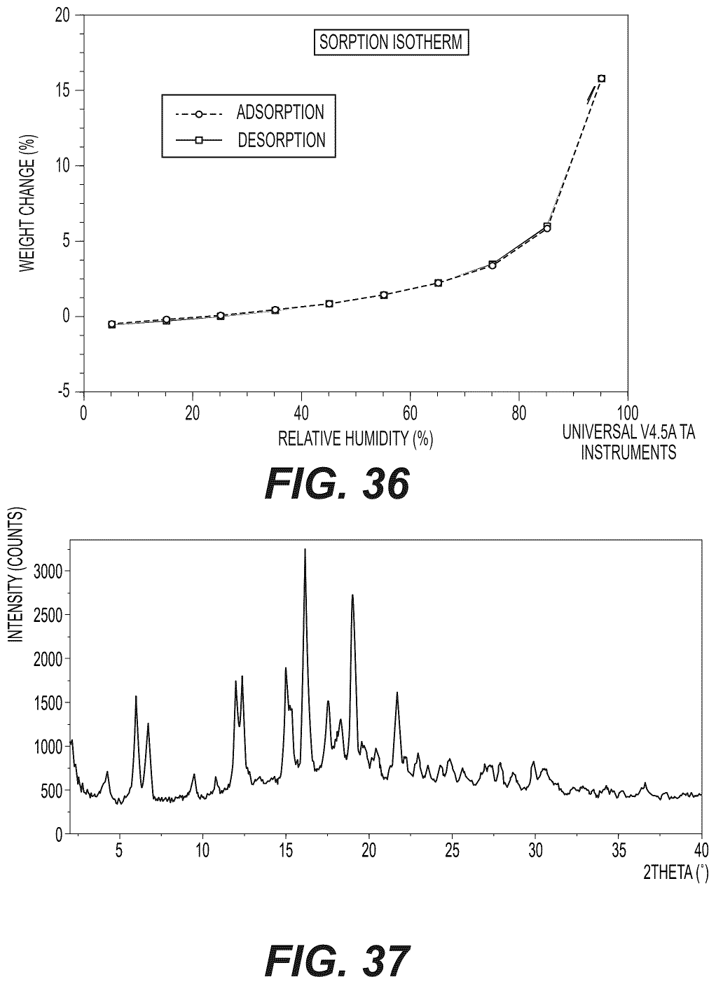

In some embodiments, the crystalline diethanolamine salt of the compound of Formula I is characterized by a DVS curve substantially as shown in shown in FIG. 36.

In some embodiments, the crystalline diethanolamine salt of the compound of Formula I has the following properties:
(a) an XRPD pattern substantially as shown in FIG. 33;
(b) a DSC thermogram substantially as shown in FIG. 34;
(c) a TGA curve substantially as shown in FIG. 35; and
(d) a DVS curve substantially as shown in FIG. 36.

In some embodiments, the crystalline diethanolamine salt of the compound of Formula I is anhydrous. In some embodiments, the crystalline diethanolamine salt of the compound of Formula f is unsolvated.

### Compound of Formula I, Piperazine Co-Crystal

In some embodiments, the compound of Formula I can be isolated as a piperazine salt or cocrystal. In some embodiments, the compound of Formula I can be isolated as a piperazine co-crystal.

In some embodiments, the compound of Formula I piperazine co-crystal is crystalline Form I (“compound of Formula I, piperazine co-crystal, Form I”).

In some embodiments, compound of Formula I, piperazine co-crystal, Form I has an XRPD profile substantially as shown in FIG. 37.

In some embodiments, compound of Formula I, piperazine co-crystal, Form I has an XRPD pattern comprising degree 2θ-reflections (+/−0.2 degrees 2θ) at 12.4°, 16.2°, and 19.0°. In some embodiments, compound of Formula I, piperazine co-crystal, Form I has an XRPD pattern comprising degree 2θ-reflections (+/−0.2 degrees 2θ) at 12.4°, 16.2°, and 19.0°, and one, two or three of the degree 2θ-reflections (+/−0.2 degrees 2θ) at 6.0°, 12.0°, and 15.0°. In some embodiments, compound of Formula I, piperazine co-crystal, Form I has an XRPD pattern comprising degree 2θ-reflections (+/−0.2 degrees 2θ) at 12.4°, 16.2°, and 19.0°, and one or two of the degree 2θ-reflections (+/−0.2 degrees 2θ) at 6.0°, 12.0°, and 15.0°. In some embodiments, compound of Formula I, piperazine co-crystal, Form I has an XRPD pattern comprising degree 2θ-reflections (+/−0.2 degrees 2θ) at 12.4°, 16.2°, and 19.0°, and one of the degree 2θ-reflections (+/−0.2 degrees 2θ) at 6.0°, 12.0°, and 15.0°. In some embodiments, compound of Formula I, piperazine co-crystal, Form I has an XRPD pattern comprising degree 2θ-reflections (+/−0.2 degrees 2θ) at 12.4°, 16.2°, and 19.0°, and two of the degree 2θ-reflections (+/−0.2 degrees 2θ) at 6.0°, 12.0°, and 15.0°. In some embodiments, compound of Formula I, piperazine co-crystal, Form I has an XRPD pattern comprising degree 2θ-reflections (+/−0.2 degrees 2θ) at 6.0°, 12.0°, 12.4°, 15.0°, 16.2°, and 19.0°. In some embodiments, compound of Formula I, piperazine co-crystal, Form I has an XRPD pattern comprising any three of the degree 2θ-reflections (+/−0.2 degrees 2θ) at 6.0°, 12.02, 12.4°, 15.0°, 16.2°, and 19.0°.

In some embodiments, compound of Formula I, piperazine co-crystal. Form I has an XRPD pattern comprising degree 2θ-reflections (+/−0.2 degrees 2θ) at 6.0°, 12.0°, 12.4°, 15.0°, 16.2°, and 19.0°, and one, two or three of the degree 2θ-reflections (+/−0.2 degrees 2θ) at 6.7°. 17.5°, and 21.7°. In some embodiments, compound of Formula I, piperazine co-crystal, Form I has an XRPD pattern comprising degree 2θ-reflections (+/−0.2 degrees 2θ) at 6.0°, 12.0°, 12.4°, 15.0°, 16.2°, and 19.0°, and one or two of the degree 2θ-reflections (+/−0.2 degrees 2θ) at 6.7°, 17.5°, and 21.7°. In some embodiments, compound of Formula I, piperazine co-crystal, Form I has an XRPD pattern comprising degree 2θ-reflections (+/−0.2 degrees 2θ) at 6.0°, 12.0°, 12.4°, 15.0°, 16.2°, and 19.0°, and one of the degree 2θ-reflections (+/−0.2 degrees 2θ) at 6.7°, 17.5°, and 21.7°. In some embodiments, compound of Formula I, piperazine co-crystal, Form I has an XRPD pattern comprising degree 2θ-reflections (+/−0.2 degrees 2θ) at 6.0°, 12.02, 12.4°, 15.0°, 16.2°, and 19.0°, and two of the degree 2θ-reflections (+/−0.2 degrees 2θ) at 6.79, 17.5°, and 21.7°. In some embodiments, compound of Formula I, piperazine co-crystal, Form I has an XRPD pattern comprising degree 2θ-reflections (+/−0.2 degrees 2θ) at 6.0°. 6.7°, 12.0°, 12.4°, 15.0°, 16.2°, 17.5°, 19.0°, and 21.7°. In some embodiments, compound of Formula I, piperazine co-crystal. Form I has an XRPD pattern comprising any three of the degree 2θ-reflections (+/−0.2 degrees 2θ) at 6.0°. 6.7°, 12.0°, 12.4°, 15.0°, 16.2°, 17.5°, 19.0°, and 21.7°. In some embodiments, compound of Formula I, piperazine co-crystal, Form I has an XRPD pattern comprising at least three of the degree 2θ-reflections (+/−0.2 degrees 2θ) at 2.1°, 4.2°. 6.0°, 6.7″, 12.0°, 12.4°, 15.0°, 16.2°, 17.5°, 19.0°, and 21.7°. In some embodiments, compound of Formula I, piperazine co-crystal. Form I has an XRPD pattern comprising at least four of the degree 2θ-reflections (+/−0.2 degrees 2θ) at 2.1°, 4.2°, 6.0°, 6.7°, 12.0°, 12.4°, 15.0°, 16.2°, 17.5°, 19.0°, and 21.7°. In some embodiments, compound of Formula I, piperazine co-crystal, Form I has an XRPD pattern comprising at least five of the degree 2θ-reflections (+/−0.2 degrees 2θ) at 2.1°, 4.2°, 6.0°, 6.7°, 12.0°, 12.4°, 15.0°, 16.2°, 17.5°, 19.0°, and 21.7°.

In some embodiments, compound of Formula I, piperazine co-crystal, Form I has an XRPD pattern comprising peaks at:

| Pos. | Rel. Int. |
|---|---|
| [°2Th. ± 0.2] | [%] |
| 2.1207 | 16.89 |
| 4.2323 | 10.94 |
| 6.0067 | 45.59 |
| 6.7108 | 32.55 |
| 9.4674 | 9.43 |
| 11.9985 | 46.39 |
| 12.3900 | 48.01 |
| 14.9970 | 47.84 |
| 16.1860 | 100.00 |
| 17.5422 | 29.24 |
| 18.3121 | 20.41 |
| 19.0220 | 78.82 |
| 21.7037 | 37.07 |
| 22.9330 | 9.25 |
| 24.6718 | 4.99 |
| 27.0287 | 6.76 |
| 28.6697 | 6.53 |
| 29.8694 | 11.17 |

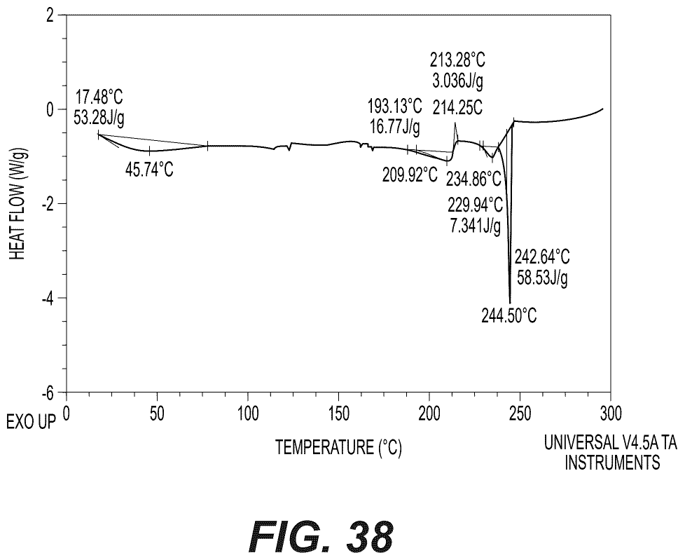

In some embodiments, compound of Formula I, piperazine co-crystal, Form I is characterized by a DSC thermogram substantially as shown in FIG. 38.

In some embodiments, compound of Formula I, piperazine co-crystal, Form I is characterized by a DSC thermogram having one, two, three, four, or all five of (i) an endothermic transition at about 17° C., (ii) an endothermic transition at about 193° C., (iii) an exothermic transition at about 213° C., (iv) an endothermic transition at about 230° C., and (v) an endothermic transition at about 243° C.

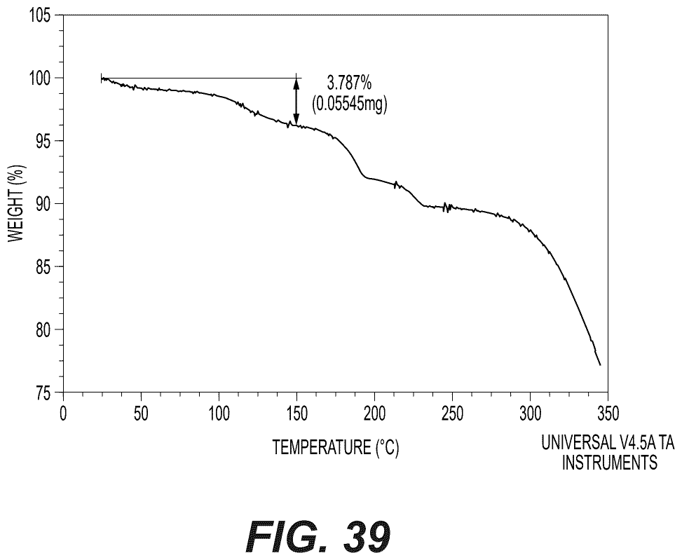

In some embodiments, compound of Formula I, piperazine co-crystal, Form I is characterized by a TGA curve substantially as shown in FIG. 39.

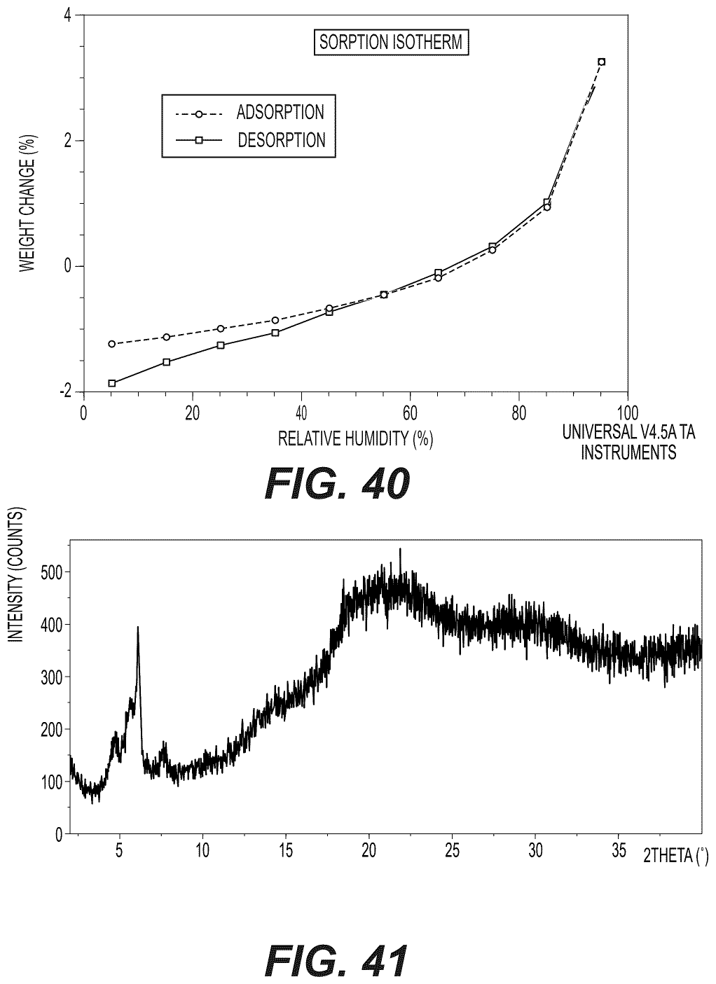

In some embodiments, compound of Formula I, piperazine co-crystal, Form I is characterized by a DVS curve substantially as shown in shown in FIG. 40.

In some embodiments, compound of Formula I, piperazine co-crystal, Form I has the following properties:
(a) an XRPD pattern substantially as shown in FIG. 37;
(b) a DSC thermogram substantially as shown in FIG. 38;
(c) a TGA curve substantially as shown in FIG. 39; and
(d) a DVS curve substantially as shown in FIG. 40.

In some embodiments, compound of Formula I, piperazine co-crystal, Form Tis hydrated. In some embodiments, compound of Formula I, piperazine co-crystal, Form I is partially or fully hydrated.

In some embodiments, the piperazine co-crystal is a hemi-piperazine co-crystal.

### Methods of Use

In some embodiments, the pharmaceutically acceptable salts, cocrystals, or crystalline forms disclosed herein are used for treating or preventing an HIV infection in a subject. In some embodiments, the pharmaceutically acceptable salts, cocrystals, or crystalline forms disclosed herein are used for treating or preventing an HIV infection in a subject at risk for infection. In some embodiments, the pharmaceutically acceptable salts, cocrystals, or crystalline forms disclosed herein are used for pre-exposure prophylaxis (PrEP) to reduce the risk of sexually acquired HIV-1. In some embodiments the pharmaceutically acceptable salts, cocrystals, or crystalline forms disclosed herein are used for pre-exposure prophylaxis to reduce HIV risk for intravenous drug users.

In some embodiments, a method for treating or preventing an HIV infection in a subject (e.g., a human), comprising administering a crystalline form, an amorphous form, a salt, or a cocrystal of the compound of Formula I disclosed herein, to the subject is disclosed.

In some embodiments, a method for inhibiting the replication of the HIV virus, treating AIDS or delaying the onset of AIDS in a subject (e.g., a human), comprising administering a crystalline form, an amorphous form, a salt, or a cocrystal of the compound of Formula I disclosed herein, to the subject is disclosed.

In some embodiments, a method for preventing an HIV infection in a subject (e.g., a human), comprising administering a crystalline form, an amorphous form, a salt, or a cocrystal of the compound of Formula I disclosed herein, to the subject is disclosed. In certain embodiments, the subject is at risk of contracting the HIV virus, such as a subject who has one or more risk factors known to be associated with contracting the HIV virus.

In some embodiments, a method for treating an HIV infection in a subject (e.g., a human), comprising administering a crystalline form, an amorphous form, a salt, or a cocrystal of the compound of Formula I disclosed herein, to the subject is disclosed.

In some embodiments, a method for treating an HIV infection in a subject (e.g., a human), comprising administering to the subject in need thereof a therapeutically effective amount of a crystalline form, an amorphous form, a salt, or a cocrystal of the compound of Formula I disclosed herein, in combination with a therapeutically effective amount of one or more (e.g., one, two, three, or four; or one or two; or one to three; or one to four) additional therapeutic agents selected from the group consisting of HIV protease inhibiting compounds. HIV non-nucleoside inhibitors of reverse transcriptase. HIV non-nucleotide inhibitors of reverse transcriptase. HIV nucleoside inhibitors of reverse transcriptase, HIV nucleotide inhibitors of reverse transcriptase, HIV integrase inhibitors, HIV nucleoside reverse transcriptase translocation inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof is disclosed.

In some embodiments, a method for treating an HIV infection in a subject (e.g., a human), comprising administering to the subject in need thereof a therapeutically effective amount of a crystalline form, an amorphous form, a salt, or a cocrystal of the compound of Formula I disclosed herein, in combination with a therapeutically effective amount of one or more (e.g., one, two, three, or four; or one or two; or one to three; or one to four) additional therapeutic agents selected from the group consisting of combination drug products for HIV, other drugs for treating HIV. HIV protease inhibitors, HIV non-nucleoside or non-nucleotide inhibitors of reverse transcriptase. HIV nucleoside or nucleotide inhibitors of reverse transcriptase, HIV integrase inhibitors, HIV non-catalytic site (or allosteric) integrase inhibitors. HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins. HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators. Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors. Complement Factor H modulators, ubiquitin ligase inhibitors, deoxycytidine kinase inhibitors, cyclin dependent kinase inhibitors, proprotein convertase PC9 stimulators. ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof is disclosed.

In some embodiments, a method for treating an HIV infection in a subject (e.g., a human), comprising administering to the subject in need thereof a therapeutically effective amount of a pharmaceutically acceptable salt of Compound 1, or a cocrystal or crystalline form thereof, in combination with a therapeutically effective amount of one or more (e.g., one, two, three, or four; or one or two; or one to three; or one to four) additional therapeutic agents selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, HIV nucleoside reverse transcriptase translocation inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof is disclosed.

In certain embodiments, a method for treating an HIV infection in a subject (e.g., a human), comprising administering to the subject in need thereof a therapeutically effective amount of a crystalline form, an amorphous form, a salt, or a cocrystal of the compound of Formula I disclosed herein, in combination with a therapeutically effective amount of one or more (e.g., one, two, three, or four; or one or two; or one to three; or one to four) additional therapeutic agents selected from the group consisting of combination drug products for HIV, other drugs for treating HIV. HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors. TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors. HIV POL protein inhibitors. Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof is disclosed.

In certain embodiments, a method for treating an HIV infection in a subject (e.g., a human), comprising administering to the subject in need thereof a therapeutically effective amount of a crystalline form, an amorphous form, a salt, or a cocrystal of the compound of Formula I disclosed herein, in combination with a therapeutically effective amount of one or more (e.g., one, two, three, or four; or one or two; or one to three; or one to four) additional therapeutic agents selected from the group consisting of combination drug products for HIV, other drugs for treating HIV. HIV protease inhibitors, HIV non-nucleoside or non-nucleotide inhibitors of reverse transcriptase. HIV nucleoside or nucleotide inhibitors of reverse transcriptase, HIV integrase inhibitors, HIV non-catalytic site (or allosteric) integrase inhibitors, and HIV nucleoside reverse transcriptase translocation inhibitors.

In certain embodiments, a pharmaceutically acceptable salt of a crystalline form, an amorphous form, a salt, or a cocrystal of the compound of Formula I disclosed herein, for use in medical therapy of an HIV infection (e.g. HIV-1 or the replication of the HIV virus (e.g. HIV-1) or AIDS or delaying the onset of AIDS in a subject (e.g., a human)) is disclosed.

In some embodiments, a pharmaceutically acceptable salt of a crystalline form, an amorphous form, a salt, or a cocrystal of the compound of Formula I disclosed herein, for use in medical therapy of an HIV infection (e.g. HIV-1 or the replication of the HIV virus (e.g. HIV-1) or AIDS or delaying the onset of AIDS in a subject (e.g., a human)) is disclosed.

In certain embodiments, a pharmaceutically acceptable salt of a crystalline form, an amorphous form, a salt, or a cocrystal of the compound of Formula I disclosed herein, for use in the manufacture of a medicament for treating an HIV infection or the replication of the HIV virus or AIDS or delaying the onset of AIDS in a subject (e.g., a human) is disclosed. One embodiment relates to a pharmaceutically acceptable salt of a crystalline form, an amorphous form, a salt, or a cocrystal of the compound of Formula I disclosed herein, for use in the prophylactic or therapeutic treatment of an HIV infection or AIDS or for use in the therapeutic treatment or delaying the onset of AIDS.

In some embodiments, a pharmaceutically acceptable salt of a crystalline form, an amorphous form, a salt, or a cocrystal of the compound of Formula I disclosed herein for use in the manufacture of a medicament for treating an HIV infection or the replication of the HIV virus or AIDS or delaying the onset of AIDS in a subject (e.g., a human) is disclosed. One embodiment relates to a crystalline form, an amorphous form, a salt, or a cocrystal of the compound of Formula I disclosed herein, for use in the prophylactic or therapeutic treatment of an HIV infection or AIDS or for use in the therapeutic treatment or delaying the onset of AIDS.

In certain embodiments, the use of a crystalline form, an amorphous form, a salt, or a cocrystal of the compound of Formula I disclosed herein, for the manufacture of a medicament for an HIV infection in a subject (e.g., a human) is disclosed. In certain embodiments, a crystalline form, an amorphous form, a salt, or a cocrystal of the compound of Formula I disclosed herein, for use in the prophylactic or therapeutic treatment of an HIV infection is disclosed.

In certain embodiments, in the methods of use, the administration is to a subject (e.g., a human) in need of the treatment. In certain embodiments, in the methods of use, the administration is to a subject (e.g., a human) who is at risk of developing AIDS.

Disclosed herein is a crystalline form, an amorphous form, a salt, or a cocrystal of the compound of Formula I disclosed herein, for use in therapy. In one embodiment, the crystalline form, a salt, or a cocrystal of the compound of Formula I disclosed herein, is for use in a method of treating an HIV infection or the replication of the HIV virus or AIDS or delaying the onset of AIDS in a subject (e.g., a human).

In some embodiments, disclosed herein is a crystalline form, an amorphous form, a salt, or a cocrystal of the compound of Formula I disclosed herein, for use in therapy. In some embodiments, a crystalline form, an amorphous form, a salt, or a cocrystal of the compound of Formula I disclosed herein, is for use in a method of treating an HIV infection or the replication of the HIV virus or AIDS or delaying the onset of AIDS in a subject (e.g., a human).

Also disclosed herein is a crystalline form, an amorphous form, a salt, or a cocrystal of the compound of Formula I disclosed herein, for use in a method of treating or preventing HIV infection in a subject in need thereof. In certain embodiments, a crystalline form, an amorphous form, a salt, or a cocrystal of the compound of Formula I disclosed herein, for use in a method of treating HIV infection in a subject in need thereof is provided. In certain embodiments, the subject in need thereof is a human who has been infected with HIV. In certain embodiments, the subject in need thereof is a human who has been infected with HIV but who has not developed AIDS. In certain embodiments, the subject in need thereof is a subject at risk for developing AIDS. In certain embodiments, the subject in need thereof is a human who has been infected with HIV and who has developed AIDS.

In one embodiment, a crystalline form, an amorphous form, a salt, or a cocrystal of the compound of Formula I disclosed herein, in combination with one or more (e.g. one, two, three, or four; or one or two; or one to three; or one to four) additional therapeutic agents as described herein for use in a method of treating or preventing HIV infection in a subject in need thereof is provided. In one embodiment, said additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors. Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof. In one embodiment, said additional therapeutic agents are selected from the group consisting of HIV protease inhibiting compounds, HIV non-nucleoside inhibitors of reverse transcriptase, HIV non-nucleotide inhibitors of reverse transcriptase. HIV nucleoside inhibitors of reverse transcriptase, HIV nucleotide inhibitors of reverse transcriptase, HIV integrase inhibitors, gp41 inhibitors. CXCR4 inhibitors, gp120 inhibitors, CCR5 inhibitors, capsid polymerization inhibitors, pharmacokinetic enhancers, and other drugs for treating HIV, and combinations thereof.

In a particular embodiment, a crystalline form, an amorphous form, a salt, or a cocrystal of the compound of Formula I disclosed herein, is provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a crystalline form, an amorphous form, a salt, or a cocrystal of the compound of Formula I disclosed herein. In certain embodiments, methods for reducing the risk of acquiring HIV (e.g., HIV-1 and/or HIV-2) comprise administration of a crystalline form, an amorphous form, a salt, or a cocrystal of the compound of Formula I disclosed herein, in combination with one or more additional therapeutic agents. In certain embodiments, methods for reducing the risk of acquiring HIV (e.g., HIV-1 and/or HIV-2) comprise administration of a crystalline form, an amorphous form, a salt, or a cocrystal of the compound of Formula I disclosed herein, and a pharmaceutically acceptable excipient.

In some embodiments, a crystalline form, an amorphous form, a salt, or a cocrystal of the compound of Formula I disclosed herein, is provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a crystalline form, an amorphous form, a salt, or a cocrystal of the compound of Formula I disclosed herein. In certain embodiments, methods for reducing the risk of acquiring HIV (e.g., HIV-1 and/or HIV-2) comprise administration of a crystalline form, an amorphous form, a salt, or a cocrystal of the compound of Formula I disclosed herein, in combination with one or more additional therapeutic agents. In certain embodiments, methods for reducing the risk of acquiring HIV (e.g., HIV-1 and/or HIV-2) comprise administration of a crystalline form, an amorphous form, a salt, or a cocrystal of the compound of Formula I disclosed herein, and a pharmaceutically acceptable excipient.

In certain embodiments, methods for reducing the risk of acquiring HIV (e.g., HIV-1 and/or HIV-2) comprise administration of a crystalline form, an amorphous form, a salt, or a cocrystal of the compound of Formula I disclosed herein, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In some embodiments, methods for reducing the risk of acquiring HIV (e.g., HIV-1 and/or HIV-2) comprise administration of a crystalline form, an amorphous form, a salt, or a cocrystal of the compound of Formula I disclosed herein, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In certain embodiments, the reduction in risk of acquiring HIV is at least about 40%, 50%, 60%, 70%, 80%, 90%, or 95%. In certain embodiments, the reduction in risk of acquiring HIV is at least about 75%. In certain embodiments, the reduction in risk of acquiring HIV is about 80%, 85%, or 90%.

In another embodiment, the use of a crystalline form, an amorphous form, a salt, or a cocrystal of the compound of Formula I disclosed herein, for the manufacture of a medicament for the treatment of an HIV infection in a human being having or at risk of having the infection is disclosed.

In some embodiments, the use of a crystalline form, an amorphous form, a salt, or a cocrystal of the compound of Formula I disclosed herein, for the manufacture of a medicament for the treatment of an HIV infection in a human being having or at risk of having the infection is disclosed.

Also disclosed herein is a crystalline form, an amorphous form, a salt, or a cocrystal of the compound of Formula I disclosed herein, for use in the therapeutic treatment or delaying the onset of AIDS.

In some embodiments, disclosed herein is a crystalline form, an amorphous form, a salt, or a cocrystal of the compound of Formula I disclosed herein, for use in the therapeutic treatment or delaying the onset of AIDS.

Also disclosed herein is a crystalline form, an amorphous form, a salt, or a cocrystal of the compound of Formula I disclosed herein, for use in the prophylactic or therapeutic treatment of an HIV infection.

In some embodiments, disclosed herein is a crystalline form, an amorphous form, a salt, or a cocrystal of the compound of Formula I disclosed herein, for use in the prophylactic or therapeutic treatment of an HIV infection.

In certain embodiments, a crystalline form, an amorphous form, a salt, or a cocrystal of the compound of Formula I disclosed herein, can be used as a research tool.

### Combination Therapy

In certain embodiments, a method for treating an HIV infection is provided, comprising administering to the human a therapeutically effective amount of a crystalline form, amorphous form, salt or co-crystal disclosed herein, in combination with a therapeutically effective amount of one, two, three, or four additional therapeutic agents. In one embodiment, a method for treating an HIV infection is provided, comprising administering to the human a therapeutically effective amount of a crystalline form, amorphous form, salt or co-crystal disclosed herein, or a pharmaceutically acceptable salt thereof, in combination with a therapeutically effective amount of one, two, three, or four additional therapeutic agents.

In one embodiment, pharmaceutical compositions comprising a crystalline form, amorphous form, salt or co-crystal disclosed herein, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a crystalline form, amorphous form, salt or co-crystal disclosed herein, in combination with a therapeutically effective amount of one, two, three, or four additional therapeutic agents which are suitable for treating an HIV infection.

In certain embodiments, a crystalline form, amorphous form, salt or co-crystal disclosed herein, is combined with one, two, three, four, or more additional therapeutic agents. In certain embodiments, a crystalline form, amorphous form, salt or co-crystal disclosed herein, is combined with one, two, three, or four additional therapeutic agents. In certain embodiments, a crystalline form, amorphous form, salt or co-crystal disclosed herein, is combined with two additional therapeutic agents. In other embodiments, a crystalline form, amorphous form, salt or co-crystal disclosed herein, is combined with three additional therapeutic agents. In further embodiments, a crystalline form, amorphous form, salt or co-crystal disclosed herein, is combined with four additional therapeutic agents. The one, two, three, four, or more additional therapeutic agents can be different therapeutic agents selected from the same class of therapeutic agents, and/or they can be selected from different classes of therapeutic agents.

In one embodiment, a crystalline form, an amorphous form, a salt, or a cocrystal of the compound of Formula I disclosed herein, in combination with one or more (e.g. one, two, three, or four; or one or two; or one to three; or one to four) additional therapeutic agents as described herein for use in a method of treating or preventing HIV infection in a subject in need thereof is provided. In one embodiment, said additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors. TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof. In one embodiment, said additional therapeutic agents are selected from the group consisting of HIV protease inhibiting compounds. HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof.

In one embodiment, a crystalline form, an amorphous form, a salt, or a cocrystal of the compound of Formula I disclosed herein, in combination with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine, is provided for use in a method of treating or preventing HIV infection in a subject in need thereof. In a particular embodiment, a crystalline form, an amorphous form, a salt, or a cocrystal of the compound of Formula I disclosed herein, in combination with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine, is provided for use in a method of treating or preventing HIV infection in a subject in need thereof.

In some embodiments, a crystalline form, an amorphous form, a salt, or a cocrystal of the compound of Formula I disclosed herein, is provided in combination with at least one additional therapeutic agent selected from the group consisting of:
(1) nucleoside reverse transcriptase translocation inhibitors (“NRTTIs”), such as 4′-Ethynyl-2-fluoro-2′-deoxyadenosine triphosphate (also known as MK-8591 and EFdA);
(2) nucleoside or nucleotide reverse transcriptase inhibitors (“NRTIs”), such as tenofovir alafenamide fumarate, tenofovir alafenamide, tenofovir alafenamide hemifumarate, GS-9131, and GS-9148;
(3) non-nucleoside or non-nucleotide reverse transcriptase inhibitors (“NNRTIs”), such as efavirenz, etravirine, rilpivirine, nevirapine, and delavirdine;
(4) protease Inhibitors (“PIs”), such as amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, and TMC-31091;
(5) integrase strand transfer inhibitors (“INSTIs”), such as Bictegravir, cabotegravir, raltegravir, and dolutegravir; and
(6) HIV capsid inhibitors, such as HIV nucleocapsid p7 (NCp7) inhibitors, HIV p24 capsid protein inhibitors, lenacapavir (GS-6207). GS-CA1. AVI-621, AVI-101, AVI-201, AVI-301, and AVI-CAN1-15 series, PF-3450074, and HIV-1 capsid inhibitors (HIV-1 infection, Shandong University).

In certain embodiments, a crystalline form, amorphous form, salt or co-crystal disclosed herein is administered with one, two, three, or four additional therapeutic agents. Co-administration of a crystalline form, amorphous form, salt or co-crystal disclosed herein disclosed herein with one, two, three, or four additional therapeutic agents generally refers to simultaneous or sequential administration of a crystalline form, amorphous form, salt or co-crystal disclosed herein and one, two, three, or four additional therapeutic agents, such that therapeutically effective amounts of the crystalline form, amorphous form, salt or co-crystal disclosed herein and the one, two, three, or four additional therapeutic agents are both present in the body of the patient. When administered sequentially, the combination may be administered in two or more administrations.

Co-administration includes administration of unit dosages a crystalline form, amorphous form, salt or co-crystal disclosed herein before or after administration of unit dosages of one, two, three, or four additional therapeutic agents. For example, a crystalline form, amorphous form, salt or co-crystal disclosed herein may be administered within seconds, minutes, or hours of the administration of the one, two, three, or four additional therapeutic agents. In some embodiments, a unit dose of a crystalline form, amorphous form, salt or co-crystal disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a crystalline form, amorphous form, salt or co-crystal disclosed herein within seconds or minutes. In other embodiments, a unit dose of a crystalline form, amorphous form, salt or co-crystal disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a crystalline form, amorphous form, salt or co-crystal disclosed herein, in combination with one or more (e.g., one, two, three, or four) additional therapeutic agents is provided.

In a specific embodiment, the kit includes a crystalline form, amorphous form, salt or co-crystal disclosed herein, an HIV nucleoside or nucleotide inhibitor of reverse transcriptase and an HIV capsid inhibitor or an HIV capsid polymerization inhibitor.

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors. HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimerie antigen receptor T-cell. CAR-T. and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors. HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors. Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drug products for HIV, other drugs for treating HIV, HIV protease inhibitors, HIV reverse transcriptase inhibitors, HIV integrase inhibitors, HIV non-catalytic site (or allosteric) integrase inhibitors. HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof. In some embodiments, the additional therapeutic agent or agents is lenacapavir

### HIV Combination Drug Products

Examples of combination drug products include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine): ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®); lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine). TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride: dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937. GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir). NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169. BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, MI-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062. US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532. MK-0536, NSC-310217, NSC-371056, NSC-48240. NSC-642710, NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV non-catalytic site, or allosteric, integrase inhibitors (NCINI) include, but are not limited to, those described in U.S. Patent Application Publication Nos. US2014221356 and US2016016973.

### HIV Infectivity Factor Inhibitors

Examples of HIV infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp 160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, Cl3hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLcc, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B. and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rey Immunol. (2018) 18 (11): 671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L): TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1. PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5. VISTA); immunoglobulin superfamily member 11 (IGSF11, VSIG3); TNFRSF14 (HVEM, CD270), TNFSF14 (HVEML): CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3. TIM3); galectin 9 (LGALS9); killer cell immunoglobulin like receptor, three Ig domains and long cytoplasmic tail 1 (KIR. CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27. CD70; CD40, CD40LG; inducible T cell costimulator (ICOS. CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR. CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18 (11): 671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591. BGB-A317 (tislelizumab), GLS-010 (WBP-3055), AK-103 (HX-008), AK-105, CS-1003, HLX-10, MGA-012, BI-754091. AGEN-2034. JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502. TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014. STI-A1015 (IMC-001), BCD-135, FAZ-053, TQB-2450. MDX1105-01, GS-4224, GS-4416, INCB086550, MAX10181, as well as multi-specific inhibitors FPT-155 (CTLA4/PD-L1/CD28). PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members. e.g., an agonist of one or more of TNFRSF1A (NCBI Gene ID: 7132), TNFRSF1B (NCBI Gene ID: 7133), TNFRSF4 (OX40, CD134; NCBI Gene ID: 7293). TNFRSF5 (CD40; NCBI Gene ID: 958), TNFRSF6 (FAS, NCBI Gene ID: 355), TNFRSF7 (CD27, NCBI Gene ID: 939). TNFRSF8 (CD30, NCBI Gene ID: 943). TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794). TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330). TNFRSF13B (CD267, NCBI Gene ID: 23495). TNFRSF13C (CD268, NCBI Gene ID: 115650). TNFRSF16 (NGFR, CD271, NCBI Gene ID: 4804), TNFRSF17 (BCMA. CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNERSF25 (DR3, NCBI Gene ID: 8718).

Examples of anti-TNFRSF4 (OX40) antibodies that can be co-administered include without limitation, MEDI6469, MEDI6383, MEDIOS62 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179. WO2017096276. WO2017096189, and WO2018089628. In some embodiments, an antibody, or fragment thereof, co-targeting TNFRSF4 (OX40) and TNFRSF18 (GITR) is co-administered. Such antibodies are described. e.g., in WO2017096179 and WO2018089628.

### Bi-and Tri-Specific Natural Killer (NK)-Cell Engagers

In various embodiments, the crystalline forms, amorphous forms, salts and co-crystals as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D. NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity). SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRIKE) include, but are not limited to, OXS-3550, HIV-TriKE, and CD16-IL-15-B7H3 TriKe.

### Indoleamine-Pyrrole-2,3-Dioxygenase (IDO1) Inhibitors

In various embodiments, the crystalline forms, amorphous forms, salts and co-crystals as described herein are combined with an inhibitor of indoleamine 2,3-dioxygenase 1 (IDO1; NCBI Gene ID: 3620). Examples of IDO1 inhibitors include without limitation, BLV-0801, epacadostat. F-001287, GBV-1012, GBV-1028, GDC-0919, indoximod, NKTR-218, NLG-919-based vaccine. PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the crystalline forms, amorphous forms, salts and co-crystals as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863. RG-7854, RG-7795, and the compounds disclosed in US20100143301 (Gilead Sciences). US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200. MCT-465, MEDI-9197, motolimod, resiquimod, GS-9688, VTX-1463, VTX-763, 3M-051, 3M-052, and the compounds disclosed in US20140045849 (Janssen). US20140073642 (Janssen), WO2014/056953 (Janssen), WO2014/076221 (Janssen), WO2014/128189 (Janssen), US20140350031 (Janssen), WO2014/023813 (Janssen). US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006. IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the crystalline forms, amorphous forms, salts and co-crystals described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the crystalline forms, amorphous forms, salts and co-crystals described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the crystalline forms, amorphous forms, salts and co-crystals as described herein are combined with an anti-TIM-3 antibody, such as TSR-022, LY-3321367, MBG-453, INCAGN-2390.

In certain embodiments, the crystalline forms, amorphous forms, salts and co-crystals herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the crystalline forms, amorphous forms, salts and co-crystals described herein are combined with an interleukin agonist, such as IL-2, IL-7, IL-15, IL-10, IL-12 agonists; examples of IL-2 agonists such as proleukin (aldeslcukin, IL-2); BC-1L (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated II-15), P-22339, and a IL-15-PD-1 fusion protein N-809; examples of IL-7 include without limitation CYT-107.

Examples of additional immune-based therapies that can be combined with the crystalline forms, amorphous forms, salts and co-crystals of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. No. 8,673,307, 9,493,549, 9,783,594, 10,239,935, US2018371086. US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195, 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492 (7427): 118-22 (2012), Horwitz et al., Proc Natl Acad Sci USA, 110 (41): 16538-43 (2013), Scheid et al., Science, 333:1633-1637 (2011), Scheid et al., Nature, 458:636-640 (2009), Eroshkin et al, Nucleic Acids Res., 42 (Database issue): DI 133-9 (2014), Mascola et al., Immunol Rev., 254 (1): 225-44 (2013), such as 2FS, 4E10. M66.6. CAP206-CH12, 10E81 (all of which bind the MPER of gp41); PG9, PG16, CH01-04 (all of which bind V1V2-glycan), 2G12 (which binds to outer domain glycan); b12, HJ16, CH103-106. VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9.783,594; and WO 2012/154312; WO2012/158948; WO 2013/086533; WO 2013/142324; WO2014/063059; WO 2014/089152. WO 2015/048462; WO 2015/103549; WO 2015/117008; WO2016/014484; WO 2016/154003: WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173 (7): 1783-1795; Sajadi et al., J Infect Dis. (2016) 213 (1): 156-64; Klein et al., Nature, 492 (7427): 118-22 (2012), Horwitz et al., Proc Natl Acad Sci USA, 110 (41): 16538-43 (2013), Scheid et al., Science, 333:1633-1637 (2011), Scheid et al., Nature, 458:636-640 (2009), Eroshkin et al., Nucleic Acids Res., 42 (Database issue): DI 133-9 (2014), Mascola et al., Immunol Rev., 254 (1): 225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59. C2F5, C4E10, C2FS+C2G12+C4E10, CAP256V2LS, 3BNC117, 3BNC117-LS, 3BNC60, DH270.1, DH270.6, DID2, 10-1074-LS. C13hmAb, GS-9722 (elipovimab), DH411-2, BG18, GS-9721, GS-9723, PGT145, PGT121, PGT-121.60, PGT-121.66, PGT122, PGT-123. PGT-124, PGT-125, PGT-126, PGT-151, PGT-130, PGT-133. PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144. PGDM1400, PGDM12, PGDM21, PCDN-33A, 2Dm2m, 4Dm2m, 6Dm2m, PGDM1400, MDX010 (ipilimumab), VRC01. VRC-01-LS, A32, 7B2, 10E8, VRC-07-523. VRC07-523LS, VRC24, VRC41.01, 10E8VLS, 3810109, 10E8v4, IMC-HIV, iMabm36, cCD4-Ig. IOMA, CAP256-VRC26.25, DRVIA7, VRC-HIVMAB080-00-AB, VRC-HIVMAB060-00-AB. P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B. 35022. ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG. TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US20140221356 (Gilead Sciences), U.S. Pat. No. 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017. DOI: 10.1128/CVI.00298-16); LNP formulated mRNA based therapeutic vaccines: LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BGSOS SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521). AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140 [delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX. HIVAX-2, N123-VRC-34.01 inducing epitope-based HIV vaccine, NYVAC-HIV-PTI, NYVAC-HIV-PT4, DNA-HIV-PT123, rAAV1-PG9DP. GOVX-B11. GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12. Pre VaxTat, AE-H, MYM-V101. CombiHIVvac, ADVAX, MYM-V201, MVA-CMDR, MagaVax, DNA-Ad5 gag/pol/nef/nev (HVTN505), MVATG-17401, ETV-01. CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01. TL-01, TUTI-16, VGX-3300, VIR-1111. IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as Derma Vir), gag-based DNA vaccine, GI-2010, gp41 HIV-1 vaccine, HIV vaccine (PIKA adjuvant), i-key/MHC class II epitope hybrid peptide vaccines. ITV-2, ITV-3, ITV-4, LIPO-5, multielade Env vaccine, MVA vaccine, Pennvax-GP, pp71-deficient HCMV vector HIV gag vaccine, rgp 160 HIV vaccine, RNActive HIV vaccine, SCB-703, Tat Oyi vaccine. TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP. INO-6145. JNJ-9220, gp145 C.6980; COD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave. TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc: tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC): TRIZIVIR® (abacavir sulfate, zidovudine, and lamivudine: ABC+AZT+3TC); rilpivirine; rilpivirine hydrochloride; atazanavir sulfate and cobicistat; atazanavir and cobicistat; darunavir and cobicistat; atazanavir: atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine: stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the crystalline forms, amorphous forms, salts and co-crystals disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, the crystalline forms, amorphous forms, salts and co-crystals disclosed herein is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, the crystalline forms, amorphous forms, salts and co-crystals disclosed herein are combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the crystalline forms, amorphous forms, salts and co-crystals described herein are combined with a gene or cell therapy regimen. Gene therapy and cell therapy include without limitation the genetic modification to silence a gene: genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the crystalline forms, amorphous forms, salts and co-crystals disclosed herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the crystalline forms, amorphous forms, salts and co-crystals disclosed herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (INKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the crystalline forms, amorphous forms, salts and co-crystals disclosed herein are combined with a population of TCR-T-cells. TCR-T-cells are engineered to target HIV derived peptides present on the surface of virus-infected cells, for example, ImmTAV.

### B-Cell Therapy

In certain embodiments, the crystalline forms, amorphous forms, salts and co-crystals disclosed herein are combined with a population of B cells genetically modified to express broadly neutralizing antibodies, such as 3BNC117 (Hartweger et al., J. Exp. Med. 2019, 1301, Moffett et al., Sci. Immunol. 4, caax0644 (2019) 17 May 2019.

The crystalline form, amorphous form, salt or co-crystal disclosed herein may be combined with one, two, three, or four additional therapeutic agents in any dosage amount of the crystalline form, amorphous form, salt or co-crystal (e.g., from 1 mg to 1000 mg of compound).

In one embodiment, kits comprising crystalline form, amorphous form, salt or co-crystal disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases. TALENs), cell therapies (such as chimeric antigen receptor T-cell, CAR-T, and engineered T cell receptors. TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV infectivity factor inhibitors. TAT protein inhibitors, HIV Nef modulators, Hck tyrosine kinase modulators, mixed lineage kinase-3 (MLK-3) inhibitors, HIV splicing inhibitors. Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

### Routes of Administration

The crystalline forms, amorphous forms, salts and co-crystals disclosed herein can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient. In certain embodiments, the crystalline forms, amorphous forms, salts and co-crystals disclosed herein can be dosed parenterally. In certain embodiments, crystalline forms, amorphous forms, salts and co-crystals disclosed herein can be dosed intravenous, subcutaneous, or intramuscular. In certain embodiments, the crystalline forms, amorphous forms, salts and co-crystals disclosed herein are dosed orally. In certain embodiments, the crystalline forms, amorphous forms, salts and co-crystals disclosed herein are dosed subcutaneously. In certain embodiments, the crystalline forms, amorphous forms, salts and co-crystals disclosed herein are dosed intramuscularly.

In some embodiments, the crystalline forms, amorphous forms, salts and co-crystals disclosed herein may be administered with a syringe suitable for administration of the compound. In some embodiments, the syringe is disposable. In some embodiments, the syringe is reusable. In some embodiments, the syringe is pre-filled with a crystalline form, amorphous form, salt or co-crystal disclosed herein.

In some embodiments, the crystalline forms, amorphous forms, salts and co-crystals disclosed herein, may be administered via injection, using an injection device. In some embodiments, the injection device is or includes a syringe, which can be employed manually, or as part of a syringe-containing injection device, such as, but not limited to, one with a needle safety shield. A wide variety of injection devices can be used, such as, for example and not limited to, a handheld or wearable autoinjector, a handheld or wearable manual injector, an on-body injector, a syrette, a jet injector, or a pen injector, each of which can be reusable or disposable.

In some embodiments, the crystalline forms, amorphous forms, salts and co-crystals disclosed herein may be administered with an auto-injector comprising a syringe. In some embodiments, the syringe is disposable. In some embodiments, the syringe is reusable. In some embodiments, the syringe is pre-filled with a crystalline form, amorphous form, salt or co-crystal disclosed herein.

### Dosing Regimen

The crystalline forms, amorphous forms, salts and co-crystals disclosed herein, may be administered to a subject in accordance with an effective dosing regimen for a desired period of time or duration, such as at least about one day, at least about one week, at least about one month, at least about 2 months, at least about 3 months, at least about 4 months, at least about 6 months, or at least about 12 months or longer. In one variation, the crystalline forms, amorphous forms, salts and co-crystals disclosed herein are administered on a daily or intermittent schedule. In one variation, the crystalline forms, amorphous forms, salts and co-crystals disclosed herein are administered on a monthly schedule. In one variation, the crystalline forms, amorphous forms, salts and co-crystals disclosed herein are administered every two months. In one variation, the crystalline forms, amorphous forms, salts and co-crystals disclosed herein are administered every three months. In one variation, the crystalline forms, amorphous forms, salts and co-crystals disclosed herein are administered every four months. In one variation, the crystalline forms, amorphous forms, salts and co-crystals disclosed herein are administered every five months. In one variation, the crystalline forms, amorphous forms, salts and co-crystals disclosed herein are administered every 6 months.

In some embodiments, the crystalline forms, amorphous forms, salts and co-crystals disclosed herein may be administered to a subject not more frequently than once per month, once every two months, once every 3 months, once every 4 months, or once every 6 months. In some embodiments, the crystalline forms, amorphous forms, salts and co-crystals disclosed herein may be subcutaneously administered to a subject not more frequently than once per month. In some embodiments, the crystalline forms, amorphous forms, salts and co-crystals disclosed herein may be subcutaneously administered to a subject not more frequently than once every two months. In some embodiments, the crystalline forms, amorphous forms, salts and co-crystals disclosed herein may be subcutaneously administered to a subject not more frequently than once every three months.

In some embodiments, the crystalline forms, amorphous forms, salts and co-crystals disclosed herein may be intramuscularly administered to a subject not more frequently than once per month. In some embodiments, the crystalline forms, amorphous forms, salts and co-crystals disclosed herein may be intramuscularly administered to a subject not more frequently than once every two months. In some embodiments, the crystalline forms, amorphous forms, salts and co-crystals disclosed herein may be intramuscularly administered to a subject not more frequently than once every three months.

In some embodiments, the crystalline forms, amorphous forms, salts and co-crystals disclosed herein may be subcutaneously or intramuscularly administered to a subject at least about every 3 months, at least about every 4 months, at least about every 5 months, or at least about every 6 months. In some embodiments, the crystalline forms, amorphous forms, salts and co-crystals disclosed herein may be administered to a subject subcutaneously, intramuscularly, or a combination thereof. In some embodiments, the crystalline forms, amorphous forms, salts and co-crystals disclosed herein may be administered at one or more initial loading doses, and one or more maintenance doses. In some embodiments, the one or more maintenance doses are administered after one month, two months, three months, or a combination thereof. In some embodiments, the initial loading dose is administered intramuscularly and the one or more maintenance doses are administered subcutaneously or intramuscularly.

In some embodiments, the crystalline forms, amorphous forms, salts, and co-crystals disclosed herein may be orally administered to a subject. In some embodiments, the crystalline forms, amorphous forms, salts, and co-crystals disclosed herein may be orally administered as an immediate release formulation.

The dosage or dosing frequency of the crystalline forms, amorphous forms, salts and co-crystals disclosed herein may be adjusted over the course of the treatment, based on the judgment of the administering physician.

The crystalline forms, amorphous forms, salts and co-crystals disclosed herein may be administered to a subject (e.g., a human) in an effective amount. In certain embodiments, the crystalline forms, amorphous forms, salts and co-crystals disclosed herein are administered once daily. In certain embodiments, the crystalline forms, amorphous forms, salts and co-crystals disclosed herein are administered once weekly. In certain embodiments, the crystalline forms, amorphous forms, salts and co-crystals disclosed herein are administered once monthly. In certain embodiments, the crystalline forms, amorphous forms, salts and co-crystals disclosed herein are administered once every two months. In certain embodiments, the crystalline forms, amorphous forms, salts and co-crystals disclosed herein are administered once every three months.

The crystalline forms, salts, and co-crystals disclosed herein may be administered in a dosage amount that is effective. For example, the dosage amount can be from 1 mg to 5000 mg, from 1 mg to 3000 mg, from 2500 mg to 5000 mg, from 1 mg to 2400 mg, or from 1 mg to 1000 mg of compound. In certain embodiments, the dosage amount is about 1, 10, 20, 30, 40, 50, 60, 70, 80, 90, 95, 100, 105, 110, 120, 130, 140, or 150 mg of the crystalline form, amorphous form, salt, or co-crystal. In certain embodiments the dosage amount is about 100, 150, 200, 250, 300, 350, 400, 450, 500, 550, 600, 650, 700, 750, 800, 850, 900, 950, or 1000 mg. In certain embodiments, the dosage amount is about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 mg. In certain embodiments, the dosage amount is about 3000, 3500, 4000, 4500, or 5000 mg.

In some embodiments, the crystalline forms, amorphous forms, salts, and co-crystals disclosed herein are administered monthly. In some embodiments, the crystalline forms, amorphous forms, salts, and co-crystals disclosed herein are administered monthly at a dose of about 150-250 mg. In some embodiments, the crystalline forms, amorphous forms, salts, and co-crystals disclosed herein are administered monthly at a dose of about 200-250 mg.

In some embodiments, the crystalline forms, amorphous forms, salts, and co-crystals disclosed herein are administered once every two months. In some embodiments, the crystalline forms, amorphous forms, salts, and co-crystals disclosed herein are administered once every two months at a dose of about 400-650 mg. In some embodiments, the crystalline forms, amorphous forms, salts, and co-crystals disclosed herein are administered once every two months at a dose of about 450-600 mg.

In some embodiments, the crystalline forms, amorphous forms, salts, and co-crystals disclosed herein are administered once every three months. In some embodiments, the crystalline forms, amorphous forms, salts, and co-crystals disclosed herein are administered once every three months at a dose of about 800-1300 mg.

### Pharmaceutical Compositions

Pharmaceutical compositions disclosed herein comprise a crystalline form, amorphous form, salt, or co-crystal disclosed herein together with one or more pharmaceutically acceptable excipients and optionally other therapeutic agents. Pharmaceutical compositions containing the crystalline form, amorphous form, salt, or co-crystal disclosed herein may be in any form suitable for the intended method of administration.

Pharmaceutical compositions comprising the crystalline form, amorphous form, salt, or co-crystal disclosed herein may be prepared with conventional carriers (e.g., inactive ingredient or excipient material) which may be selected in accord with ordinary practice. Tablets may contain excipients including glidants, fillers, binders and the like. Oral suspensions or solution formulations may be prepared in sterile form, and when intended for delivery by other than oral administration (e.g., injectable or parenteral) generally may be isotonic. All compositions may optionally contain excipients such as those set forth in the Rowe et al, Handbook of Pharmaceutical Excipients, 5th edition, American Pharmacists Association, 1986. Excipients can include ascorbic acid and other antioxidants, chelating agents such as EDTA, carbohydrates such as dextrin, hydroxyalkylcellulose, hydroxyalkylmethylcellulose, stearic acid and the like.

While it is possible for the active ingredient (e.g. the crystalline form, amorphous form, salt, or co-crystal disclosed herein) to be administered alone, it may be preferable to present the active ingredient as pharmaceutical compositions. The compositions, both for veterinary and for human use, comprise at least the crystalline form, amorphous form, salt, or co-crystal disclosed herein together with one or more acceptable carriers and optionally other therapeutic ingredients. In one embodiment, the pharmaceutical composition comprises a crystalline form, amorphous form, salt, or co-crystal disclosed herein, a pharmaceutically acceptable excipient, and a therapeutically effective amount of one or more (e.g., one, two, three, or four; or one or two; or one to three; or one to four) additional therapeutic agents as defined hereinbefore. In one embodiment, the pharmaceutical composition comprises a crystalline form, amorphous form, salt, or co-crystal disclosed herein, a pharmaceutically acceptable excipient, and one other therapeutic ingredient. The carrier(s) are “acceptable” in the sense of being compatible with the other ingredients of the composition and physiologically innocuous to the recipient thereof.

The compositions include those suitable for various administration routes. The compositions may conveniently be presented in unit dosage form and may be prepared by any of the methods well known in the art of pharmacy. Such methods include the step of bringing into association the active ingredient (e.g. the crystalline form, amorphous form, salt, or co-crystal disclosed herein) with one or more inactive ingredients (e.g., a carrier, pharmaceutical excipient, etc.). The compositions may be prepared by uniformly and intimately bringing into association the active ingredient with liquid carriers or finely divided solid carriers or both, and then, if necessary, shaping the product. Techniques and formulations generally are found in Remington: The Science and Practice of Pharmacy, 21^{st} Edition, Lippincott Williams and Wilkins, Philadelphia, Pa., 2006.

Compositions described herein that are suitable for oral administration may be presented as discrete units (a unit dosage form) including but not limited to capsules, cachets or tablets each containing a predetermined amount of the active ingredient. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium carbonate or sodium carbonate, lactose, lactose monohydrate, croscarmellose sodium, povidone, calcium phosphate or sodium phosphate; granulating and disintegrating agents, such as maize starch, or alginic acid; binding agents, such as cellulose, microcrystalline cellulose, starch, gelatin or acacia; lubricating agents, such as magnesium stearate, stearic acid or talc; and glidants, such as colloidal silicon dioxide. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, disclosed herein are oral dosage forms (e.g., tablets), which may be prepared from hot melt extrusion or spray-drying dispersion (SDD) technologies. In some embodiments, the oral dosage forms are prepared by spray-drying dispersion with one or more polymers (e.g., copovidone, povidone, hypromellose, hypromellose phthalate, hypromellose acetate succinate, cellulose acetate phthalate, methacrylic acid-ethyl acrylate copolymer, or combinations thereof).

In some embodiments, disclosed herein are hard capsules filled with powder, beads, or granules containing the active ingredient in admixture with non-toxic pharmaceutically acceptable excipient which are suitable for manufacture of hard or soft capsules. These excipients may be, for example, inert diluents, such as calcium carbonate or sodium carbonate, lactose, lactose monohydrate, croscarmellose sodium, povidone, calcium phosphate or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc.

In some embodiments, disclosed herein are hard or soft capsules filled with liquid or semi-solid mixtures containing the active ingredient in admixture with non-toxic pharmaceutically acceptable excipient which are suitable for manufacture of hard or soft capsules. These excipients may be, for example, solubilizing oils such as maize oil, sesame oil, or corn oil; medium chain triglycerides and related esters, such as, derivitized palm kernel oil or coconut oil; self-emulsifying lipid systems (SEDDS or SMEDDS), such as caprylic triglyceride or propylene glycol monocaprylate; viscosity modifiers, such as, cetyl alcohol, steryl alcohol, glycerol stearate; and solubilizing agents and surfactants, such as polyethylene glycol, propylene glycol, glycerin, ethanol, polyethoxylated castor oil, poloxamers, or polysorbates.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are known to the person skilled in the art of parenteral formulation and may be found e.g., in Handbook of Pharmaceutical Excipients (eds. Rowe, Sheskey & Quinn), 6th edition 2009.

In certain embodiments, the composition is disclosed as a solid dosage form, including a solid injectable dosage form, such as a solid depot form.

The amount of active ingredient that may be combined with the inactive ingredients to produce a dosage form may vary depending upon the intended treatment subject and the particular mode of administration. For example, in some embodiments, a dosage form for oral or parenteral administration to humans may contain approximately 1 to 5000 mg of active material formulated with an appropriate and convenient amount of carrier material (e.g., inactive ingredient or excipient material). In certain embodiments, the carrier material varies from about 5 to about 95% of the total compositions (weight:weight).

It should be understood that in addition to the ingredients particularly mentioned above the compositions of these embodiments may include other agents conventional in the art having regard to the type of composition in question, for example those suitable for oral administration may include flavoring agents.

In certain embodiments, a composition comprising an active ingredient disclosed herein in one variation does not contain an agent that affects the rate at which the active ingredient is metabolized. Thus, it is understood that compositions comprising a crystalline form, amorphous form, salt, or co-crystal disclosed herein, in certain embodiments do not comprise an agent that would affect (e.g., slow, hinder or retard) the metabolism of the crystalline form, amorphous form, salt, or co-crystal, or any other active ingredient administered separately, sequentially or simultaneously with the salt, cocrystal, or crystalline form. It is also understood that any of the methods, kits, articles of manufacture and the like detailed herein in certain embodiments do not comprise an agent that would affect (e.g., slow, hinder or retard) the metabolism of a crystalline form, amorphous form, salt, or co-crystal, or any other active ingredient administered separately, sequentially or simultaneously with the crystalline form, amorphous form, salt, or co-crystal.

### Kits and Articles of Manufacture

The present disclosure relates to a kit comprising a crystalline form, amorphous form, salt, or co-crystal disclosed herein. In one embodiment, the kit may comprise one or more additional therapeutic agents as described herein before. The kit may further comprise instructions for use, e.g., for use in inhibiting an HIV reverse transcriptase, such as for use in treating an HIV infection or AIDS or as a research tool. The instructions for use are generally written instructions, although electronic storage media (e.g., magnetic diskette or optical disk) containing instructions are also acceptable.

The present disclosure also relates to a pharmaceutical kit comprising one or more containers comprising crystalline form, amorphous form, salt, or co-crystal disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

In some embodiments, the present disclosure also relates to a pharmaceutical kit comprising one or more containers comprising a crystalline form, amorphous form, salt, or co-crystal disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

Also disclosed are articles of manufacture comprising a unit dosage of a crystalline form, amorphous form, salt, or co-crystal, disclosed herein in suitable packaging for use in the methods described herein. Suitable packaging is known in the art and includes, for example, vials, vessels, ampules, bottles, jars, flexible packaging, prefilled syringes, empty disposable syringes, autoinjectors, needles, and the like. An article of manufacture may further be sterilized and/or sealed.

### EXAMPLES

### General Methods

X-ray powder diffraction (XRPD) analysis was conducted on a diffractometer (PANalytical XPERT-PRO, PANalytical B. V . . . . Almelo, Netherlands) using copper radiation (Cu Kα, λ=1.541874 Å). Samples were spread evenly on a zero background sample plate. The generator was operated at a voltage of 45 kV and amperage of 40 mA. Slits were Soller 0.02 rad, antiscatter 1.0°, and divergence. Scans were performed from 2 to 40° 20 with a 0.0167 step size. Data analysis was performed using X′Pert Data Viewer V1.2d (PANalytical B.V., Almelo, Netherlands). X-ray powder diffraction analysis was also conducted on a diffractometer (Rigaku MiniFlex, Rigaku Corporation, Beijing, China) using copper radiation (Cu Kα, λ=1.541874 Å). Samples were spread evenly on a zero background sample plate. The generator was operated at a voltage of 40 kV and amperage of 15 mA. Scans were performed from 2 to 40° 2θ with a 0.050 degree step size and a speed of 2 degrees/minute. Data analysis was also performed using X′Pert Data Viewer V1.2d (PANalytical B.V., Almelo, Netherlands). Peaks in the XRPD patterns are classified herein as Tier 1, Tier 2, and Tier 3 according to relative peak intensity, with Tier 1 including the strongest intensity peaks.

Differential Scanning calorimetry (DSC) was run on a Q2000 (TA Instruments, New Castle, DE) by loading 1-10 mg of material into a crimped or open Tzero standard aluminum pan and heating the sample at 10° C./min from 20 to 300° C. or above. The sample and reference pans were under a 50 mL/min nitrogen purge. Data analysis was completed using Universal Analysis 2000 Version 4.5A (TA Instruments, New Castle, DE).

Thermogravimetric analysis (TGA) was used to evaluate sample weight loss as a function of temperature on either a Q5000 or Q500 (TA Instruments, New Castle, DE), by loading 1-10 mg of material onto a weigh pan and heating the sample to 350° C. or above at a rate of 10° C./min. The sample and reference pans were under a 60 mL/min and 40 mL/min nitrogen purge, respectively. Data analysis was completed using Universal Analysis 2000 Version 4.5A (TA Instruments, New Castle, DE).

Hygroscopicity was studied using dynamic vapor sorption (DVS, TA Q5000 SA, TA Instruments, New Castle, DE or DVS, DVS Intrinsic, Surface Measurement Systems, London, UK). A sample (1-20 mg) was placed in an aluminum DVS pan and loaded on the sample side of the twin pan balance. The water sorption and desorption were studied as a function of relative humidity (RH) at 25° C. In 10% RH increments, the relative humidity was increased from 5% RH to 95% RH and then decreased back to 5%. Each relative humidity increment had an equilibration time of 180 minutes, unless weight change % was less than 0.002% in 30 minutes. Data analysis was performed using Universal Analysis 2000 Version 4.7A (TA Instruments, New Castle, DE) for TA DVS runs and Microsoft Excel for SMS DVS runs.

### Example 1. Compound of Formula I, Form 1

### Example 1a

Compound of Formula I, Form I is an unsolvated phase. It was isolated when amorphous compound of Formula I was slurried separately in the following solvent systems at room temperature for about 14 days: Acetone, MeCN, EtOH, EtOAc, IPA, IPOAc, MeTHE, toluene, THF, and water.

### Example 1b

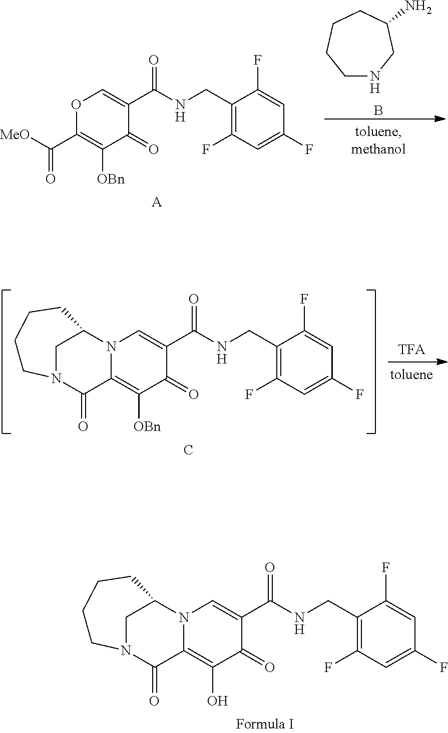

Compound of Formula I, Form I was also isolated when methyl 3-(benzyloxy)-4-oxo-5-((2,4,6-trifluorobenzyl) carbamoyl)-4H-pyran-2-carboxylate (A) (1.0 equiv, scaling factor), toluene (5.4 volumes), (S)-azepan-3-amine (B) (1.1 equiv), and methanol (0.6 volumes) were charged to a reactor. The mixture was agitated at about 55° C. until the reaction was deemed complete. The mixture was then concentrated under reduced pressure. Toluene (4.0 volumes) was charged, and the mixture was concentrated again under reduced pressure. Toluene (4.0 volumes) and trifluoroacetic acid (5.0 equiv) were then charged, and the mixture was agitated at about 55° C. until the reaction was deemed complete. The mixture was then concentrated under reduced pressure. Toluene (4.0 volumes) was charged, and the mixture was again concentrated under reduced pressure. 2-Propanol (2.0 volumes) was charged, and the mixture was agitated at about 55° C. for about 1 hour. Additional 2-propanol (10 volumes) was then charged over about 1 hour, then the mixture was cooled to about 25° C. and agitated for about 16 hours. The slurry was filtered and resulting cake rinsed with 2-propanol (3.0 volumes), rinsed twice with ethanol (3.0 volumes), then dried to afford the compound of Formula I, Form I.

### Example 1c

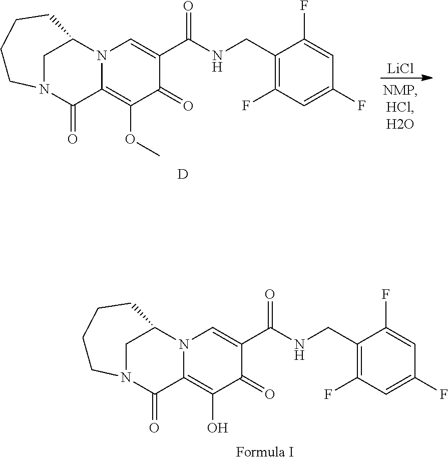

Compound of Formula I, Form I was also isolated when (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (D) (1.00 equiv, scaling factor), lithium chloride (3.0 equiv), and N-methyl-2-pyrolidine (3.0 volumes) were charged to a reactor. The mixture was heated to about 100° C. and agitated until the reaction was deemed complete. The reaction mixture was then charged dropwise to another reactor containing 1 M hydrochloric acid (3.0 volumes) at about 2° C. The slurry was then agitated at about 25° C. for about 16 hours. The slurry was then filtered and rinsed twice with water (3.0 volumes), then rinsed twice with 2-propanol (3.0 volumes), and finally dried to afford the compound of Formula I, Form I.

### Example 1d

Compound of Formula I, Form I was also isolated when methyl 3-(benzyloxy)-4-oxo-5-((2,4,6-trifluorobenzyl) carbamoyl)-4H-pyran-2-carboxylate (A) (1.00 equiv, scaling factor), (S)-azepan-3-amine (B) (1.1 equiv.), sodium bicarbonate (2.15 equiv.), and methanol (6.0 volumes) to a reactor. The mixture was heated to about 60° C. and agitated until the reaction was deemed complete. Ethyl acetate (10.0 volumes) and saturated aqueous ammonium chloride (5.0 volumes) were charged and the mixture agitated for about 15 minutes. The phases were allowed to separate, and the lower layer was discarded. Water (5.0 volumes) was charged, and the mixture agitated for about 15 minutes. The phases were allowed to separate, and the lower layer was discarded. The mixture was then concentrated under reduced pressure. Toluene (4.0 volumes) was charged, and the mixture was concentrated again under reduced pressure. Toluene (4.0 volumes) and trifluoroacetic acid (4.0 volumes) was charged. The mixture was heated to about 60° C. and agitated until the reaction was deemed complete. The mixture was concentrated under reduced pressure. Ethyl acetate (2.0 volumes) was charged, and the mixture was seeded with Form I at room temperature. Heptane (2.0 volumes) was then charged, and the mixture agitated for about 30 minutes. Additional heptane (4.0 volumes) was charged, and the mixture agitated for about 12 h. The slurry was then filter and rinsed with ethyl acetate (1.0 volume), and dried to afford compound of Formula I, Form I.

### Example 1e

The compound of Formula I, Form I was also isolated when the compound of Formula I, sodium salt Form 1 (1.00 equiv, sealing factor) and ethanol (10.0 volumes) were charged to a reactor. Glacial acetic acid (2.0 equiv) was charged over about 1 h during which the mixture was agitated and subjected to high sheer wet milling at about 15,000 rpm with recirculation. Agitation and wet-milling was continued for about 30 minutes and the mixture was further agitated for about 3 hours. The slurry was then filtered, rinsed twice with ethanol (1.0 volumes), and dried at about 50° C. for about 20 hours.

The Compound of Formula I, Form I XRPD pattern is shown in FIG. 1 and is characterized by Tier 1 reflections at 17.8, 22.0, 25.4° 2θ, but also Tier 2 at 15.6, 23.1, 29.2° 2θ, and Tier 3 at 11.1, 12.6, 17.3° 2θ. A list of 2-theta peaks is provided below:

Peak Table

| Pos. | Rel. Int. |
|---|---|
| [°2Th. ± 0.2] | [%] |
| 10.2051 | 17.91 |
| 11.0588 | 34.41 |
| 12.6118 | 31.59 |
| 14.5193 | 17.32 |
| 15.6463 | 41.62 |
| 17.2528 | 38.69 |
| 17.8322 | 100.00 |
| 19.6384 | 14.92 |
| 20.7623 | 16.29 |
| 21.9574 | 96.32 |
| 23.1489 | 44.24 |
| 24.2050 | 21.41 |
| 25.0185 | 40.07 |
| 25.3612 | 84.53 |
| 26.2356 | 11.77 |
| 27.7314 | 10.03 |
| 28.6533 | 34.12 |
| 29.1593 | 49.06 |
| 30.5358 | 20.39 |
| 34.8604 | 22.44 |
| 38.5013 | 12.61 |

The DSC curve is shown in FIG. 2 and displays one endothermic transition at about 246° C. The TGA curve is shown in FIG. 3 and indicates that the phase is unsolvated. The dynamic vapor sorption curve is shown in FIG. 4 and the data indicate that the form absorbs about 0.097% of water up to 95% RH at 25° C. The material was found to not have changed form post experiment.

Single crystal data was collected on the compound of Formula I, Form I and the data are summarized in Table 1 and FIG. 5.

The crystal system is monoclinic and the space group is P21. The cell parameters and calculated volume are: a=8.94841 (12) Å, b=8.58582 (16) Å, c=12.59306 (18) Å, α=90°, β=103.2526 (13), γ=90°. V=941.75 (3) Å³. The molecular weight is 421.37 g mol⁻¹ with Z=2, resulting in a calculated density of 1.486 g cm⁻³.

TABLE 2
Crystal Data and Data Collection Parameters
for Formula I, Form I

| | |
|---|---|
| Empirical formula | C₂₀H₁₈F₃N₃O₄ |
| Formula weight (g mol⁻¹) | 421.37 |
| Temperature (K) | 300.1(2) |
| Wavelength (Å) | 1.54184 |
| Crystal system | monoclinic |
| Space group | P2₁ |
| Unit cell parameters | |
| a = 8.94841(12) Å | α = 90° |
| b = 8.58582(16) Å | β = 103.2526(13)° |
| c = 12.59306(18) Å | γ = 90° |
| Unit cell volume (Å³) | 941.75(3) |
| Cell formula units, Z | 2 |
| Calculated density (g cm⁻³) | 1.486 |
| Absorption coefficient (mm⁻¹) | 1.070 |
| F(000) | 436 |
| Crystal size (mm³) | 0.21 × 0.08 × 0.02 |
| Reflections used for cell measurement | 6168 |
| θ range for cell measurement | 5.0740°-77.2490° |
| Total reflections collected | 9769 |
| Index ranges | −10 ≤ h ≤ 11; |
| | −10 ≤ k ≤ 9; −15 ≤/≤ 15 |
| θ range for data collection | θₘᵢₙ = 5.078°, |
| | θₘₐₓ = 77.513° |
| Completeness to θₘₐₓ | 97.8% |
| Completeness to θ_{full} = 67.684° | 99.8% |
| Absorption correction | multi-scan |
| Transmission coefficient range | 0.891-1.000 |
| Refinement method | full matrix |
| | least-squares on F² |
| Independent reflections | 3339 [Rᵢₙₜ = 0.0233, |
| | R_{σ} = 0.0230] |
| Reflections [ I > 2σ(I) ] | 3062 |
| Reflections/restraints/parameters | 3339/1/343 |
| Goodness-of-fit on F² | S = 1.05 |
| Final residuals [ I > 2σ(I) ] | R = 0.0303, R_{w} = 0.0811 |
| Final residuals [ all reflections ] | R = 0.0331, R_{w} = 0.0835 |
| Largest diff. peak and hole (e Å⁻³) | 0.104, −0.149 |
| Max/mean shift/standard uncertainty | 0.000/0.000 |
| Absolute structure determination | Flack |
| | parameter: 0.00(8) |
| | Hooft parameter: |
| | 0.03(7) |
| | Friedel coverage: 66.4% |

### Example 2. Compound of Formula I, Form II

Compound of Formula I, Form II is a metastable methanol solvated phase. It was isolated when compound of Formula I, Form I was slurried in methanol for about 60 days and identified by x-ray of the wet cake. Upon drying compound of Formula I, Form II under ambient conditions, it was found to convert to compound of Formula I, Form III.

The XRPD pattern for the compound of Formula I, Form II is shown in FIG. 6 and is characterized by Tier 1 reflections at 9.5, 10.9, 21.2° 2θ, but also Tier 2 at 14.7, 17.8, 18.9° 2θ, and Tier 3 at 6.3, 28.0, 28.7° 2θ. A list of 2-theta peaks is provided below:

Peak Table

| Pos. | Rel. Int. |
|---|---|
| [°2Th.] | [%] |
| 5.4610 | 17.91 |
| 6.3322 | 25.41 |
| 8.9092 | 23.95 |
| 9.4542 | 100.00 |
| 10.9298 | 69.05 |
| 13.2035 | 4.15 |
| 14.7383 | 62.27 |
| 15.6982 | 15.85 |
| 16.3292 | 15.33 |
| 17.2677 | 16.89 |
| 17.7594 | 48.53 |
| 18.0435 | 39.97 |
| 18.8815 | 55.12 |
| 19.4545 | 19.10 |
| 21.2189 | 64.10 |
| 23.1999 | 32.03 |
| 23.9297 | 32.13 |
| 25.7520 | 21.42 |
| 27.0226 | 14.90 |
| 27.4848 | 12.72 |
| 28.0375 | 43.04 |
| 28.6660 | 40.58 |
| 29.4663 | 20.64 |
| 30.1798 | 8.24 |
| 30.6728 | 18.29 |

### Example 3. Compound of Formula I, Form III

### Example 3a

Compound of Formula I, Form III is a methanol solvated phase. It was isolated when amorphous compound of Formula I was slurried in MeOH at room temperature for about 14 days and isolated as a dry cake.

### Example 3b

Compound of Formula I, Form III was also made when Form II was dried under ambient conditions.

### Example 3c

Compound of Formula I, Form III was also isolated when compound of Formula I, sodium salt, Form I (1.00 equiv, scaling factor), methanol (5.0 volumes), and ethyl acetate (5.0 volumes) were charged to a reactor. The mixture was heated to about 40° C. and glacial acetic acid (1.6 equiv) was charged. The mixture was then heated to about 60° C., agitated for about 30 minutes, and then cooled to about 20° C. over about 2 hours. The slurry was agitated for about 16 hours at about 20° C. The slurry was then filtered, rinsed with 50% methanol in ethyl acetate (2.0 volumes), and dried at about 50° C. for about 24 h to afford Compound of Formula I, Form III.

The compound of Formula I, Form III XRPD pattern is shown in FIG. 7 and is characterized by Tier 1 reflections at 15.8, 17.3, 26.4° 2θ, but also Tier 2 at 14.9, 18.1, 21.8° 2θ, and Tier 3 at 5.8, 9.7, 10.5° 2θ. A list of 2-theta peaks is provided below:

Peak Table

| Pos. | Rel. Int. |
|---|---|
| [°2Th.] | [%] |
| 5.7681 | 24.93 |
| 9.7205 | 22.56 |
| 10.5053 | 40.02 |
| 11.5440 | 29.19 |
| 14.9183 | 53.73 |
| 15.8074 | 64.25 |
| 17.2791 | 100.00 |
| 18.0835 | 51.41 |
| 19.4465 | 36.71 |
| 21.7763 | 55.41 |
| 23.7336 | 8.60 |
| 24.7317 | 5.43 |
| 26.3864 | 88.10 |
| 29.4755 | 23.36 |
| 30.2145 | 37.76 |
| 30.7309 | 18.01 |

The DSC curve is shown in FIG. 8 and displays an endothermic transition at about 156° C., an endothermic transition at about 180° C., an exothermic transition at about 186° C., and an endothermic transition at about 247° C. The TGA curve is shown in FIG. 9 and indicates that the phase is solvated. The dynamic vapor sorption curve is shown in FIG. 10 and the data indicate that the form absorbs about 0.87% of water up to 95% RH at 25° C. The material was found to not have changed form post experiment.

### Example 4. Compound of Formula I, Form IV

Compound of Formula I, Form IV is a hydrated phase. It was isolated when 500 μL of a DMSO stock solution (˜50 mg/mL, compound of Formula I) was added to either 50 mL of 0.1 wt/v % poloxamer 188 in simulated gastric fluid, 0.1 wt/v % poloxamer 338 in simulated gastric fluid, 0.1 wt/v % tocopheryl polyethylene glycol succinate in simulated gastric fluid, or 0.1 wt/v % tween 80 in simulated gastric fluid and then stirred at room temperature for around 24 hours.

The compound of Formula I, Form IV XRPD pattern is shown in FIG. 11 and is characterized by Tier 1 reflections at 4.1, 12.3, 16.4° 2θ, but also Tier 2 at 13.7, 23.4, 25.4° 2θ, and Tier 3 at 8.1, 18.9, 27.4° 2θ. A list of 2-theta peaks is provided below:

Peak Table

| Pos. | Rel. Int. |
|---|---|
| [°2Th.] | [%] |
| 4.0905 | 61.21 |
| 8.1684 | 25.79 |
| 12.2546 | 85.48 |
| 13.7183 | 59.27 |
| 15.5882 | 25.92 |
| 16.3620 | 100.00 |
| 17.4922 | 23.39 |
| 18.4025 | 6.79 |
| 18.9112 | 34.07 |
| 21.3775 | 18.03 |
| 22.1915 | 9.88 |
| 23.3990 | 54.83 |
| 24.6191 | 18.60 |
| 25.4026 | 52.81 |
| 27.4476 | 29.32 |
| 28.4581 | 15.08 |
| 28.8361 | 16.20 |
| 31.4865 | 14.90 |
| 33.0815 | 17.71 |
| 35.1201 | 13.63 |

The DSC curve is shown in FIG. 12 and displays an endothermic transition at about 33° C., an exothermic transition at about 86° C., and an endothermic transition at about 244° C. The TGA curve is shown in FIG. 13 and indicates that the phase is hydrated. The dynamic vapor sorption curve is shown in FIG. 14, and the data indicate that the form absorbs about 3.5% of water up to 95% RH at 25° C. The material was found to not have changed form post experiment.

### Example 5. Compound of Formula I, Amorphous

Compound of Formula I, amorphous was isolated when approximately 20 g of compound of Formula I, Form I was dissolved in dichloromethane (˜250 ml) at room temperature and the solution was spray dried to yield a powder. The inlet temperature for the spray drier was about 50° C., while the outlet temperature as about 35° C.

The XRPD pattern for the compound of Formula I, amorphous is shown in FIG. 15 and is characterized by a broad hump. The DSC curve is shown in FIG. 16 indicating a glass transition around 110° C.

### Example 6. Compound of Formula I, Sodium Salt

### Example 6a

Sodium salt of the compound of Formula I is an unsolvated phase. It was isolated when about 1000 mg of the compound of Formula I, Form I was placed in a vial with ˜ 10 mL of EtOH. Next, about 96 mg of solid NaOH was added along with about 1 mL of water and the sample was sonicated for about 60 minutes to yield a precipitate. Next, about 10 mL of EtOH was added and the sample was stirred at room temperature for about 3 days then filtered and air dried.

### Example 6b

Sodium salt of the compound of Formula I was also isolated when compound of Formula I, Form I (1.0 equiv, scaling factor) and ethanol (10 volumes) were charged to a reactor. The mixture was heated to about 75° C. and 25 wt % sodium hydroxide in water (1.1 equiv) was charged to the reactor, followed by rinsing forward with water (0.2 volumes). The reaction mixture was then agitated for about 30 minutes, cooled to about 20° C. over about 4 hours, and then agitated for about 18 hours. The slurry was filtered, rinsed twice with 95% ethanol in water (2.0 volumes), and dried at about 50° C.

The sodium salt XRPD pattern is shown in FIG. 17 and is characterized by Tier 1 reflections at 6.5, 20.2, 23.5° 2θ, but also Tier 2 at 14.3, 17.2, 19.2° 2θ, and Tier 3 at 13.0, 19.8, 30.8° 2θ. A list of 2-theta peaks is provided below:

Peak Table

| Pos. | Rel. Int. |
|---|---|
| [°2Th.] | [%] |
| 6.4582 | 100.00 |
| 9.1357 | 7.98 |
| 10.9197 | 5.37 |
| 12.9859 | 9.48 |
| 14.3397 | 23.23 |
| 16.6524 | 3.41 |
| 17.1777 | 21.24 |
| 18.2116 | 8.54 |
| 18.7336 | 3.89 |
| 19.2149 | 15.37 |
| 19.7692 | 11.99 |
| 20.2415 | 39.53 |
| 20.9268 | 6.60 |
| 21.8961 | 6.32 |
| 22.5979 | 3.26 |
| 23.1770 | 9.59 |
| 23.4848 | 34.81 |
| 26.4924 | 4.82 |
| 27.2889 | 6.52 |
| 28.4243 | 7.38 |
| 28.8215 | 8.79 |
| 30.1002 | 9.22 |
| 30.7739 | 11.53 |
| 32.8031 | 6.89 |
| 34.1696 | 3.24 |

The DSC curve is shown in FIG. 18 and displays an endothermic transition at about 371° C. and an exothermic transition at about 374° C. The TGA curve is shown in FIG. 19 and indicates that the phase is unsolvated. The dynamic vapor sorption curve is shown in FIG. 20 and the data indicate that the form absorbs about 0.48% of water up to 95% RH at 25° C. The material was found to not have changed form post experiment.

### Example 7. Compound of Formula I, Potassium Salt, Form I

Compound of Formula I, potassium salt, Form I is a hydrated phase. It was isolated when ˜897 mg of Compound of Formula I, Form I was placed in a vial with about 3 mL of EtOH. ˜ 120 mg of solid KOH was added, and the sample was sonicated for about 60 minutes to yield a thin precipitate. The sample was stirred at room temperature for about 3 days to yield a thick precipitate, to which about 15 mL of EtOH was added and stirred for several hours. About 20 mL of heptane was then added, and sample stirred for about 1 day. The precipitate was then filtered and air dried.

The potassium salt Form I XRPD pattern is shown in FIG. 21 and is characterized by Tier 1 reflections at 6.6, 19.7, 23.9° 2θ, but also Tier 2 at 9.0, 28.3, 28.8° 2θ, and Tier 3 at 15.2, 22.9, 24.7° 2θ. A list of 2-theta peaks is provided below:

Peak Table

| Pos. | Rel. Int. |
|---|---|
| [°2Th.] | [%] |
| 5.1492 | 21.58 |
| 6.5914 | 46.20 |
| 9.0435 | 37.69 |
| 10.0631 | 8.48 |
| 13.7803 | 24.04 |
| 15.2024 | 28.15 |
| 16.9249 | 19.94 |
| 18.2685 | 25.25 |
| 19.7188 | 100.00 |
| 20.7272 | 19.45 |
| 21.8640 | 20.74 |
| 22.9141 | 27.99 |
| 23.9132 | 46.53 |
| 24.6611 | 34.57 |
| 26.7792 | 25.53 |
| 28.2922 | 41.93 |
| 28.7775 | 45.47 |
| 29.8952 | 13.62 |
| 31.0161 | 13.16 |
| 32.0096 | 16.24 |
| 33.3248 | 13.97 |
| 35.1042 | 14.69 |

The DSC curve is shown in FIG. 22 and displays an endothermic transition at about 18° C., an endothermic transition at about 84° C., a baseline shift at 228° C., and an exothermic transition at about 300° C. The TGA curve is shown in FIG. 23 and indicates that the phase is hydrated. The dynamic vapor sorption curve is shown in FIG. 24 and the data indicate that the form absorbs about ˜60% of water up to 95% RH at 25° C. The material was found to have converted to an amorphous form post experiment.

### Example 8. Compound of Formula I, Potassium Salt, Form II

Compound of Formula I, potassium salt Form II is a hydrated phase. It was isolated when Compound of Formula I, Form I (1.00 equiv, scaling factor), ethanol (7.0 volumes), and 2-propanol (3.0 volumes) were charged to a reactor. The mixture was heated to about 75° C. and 33 wt % potassium hydroxide in water (1.1 equiv) was charged. The reaction mixture was agitated for about 30 minutes and cooled to about 60° C. 2-propanol (2.0 volumes) was charged to a reactor and the mixture was concentrated under reduced pressure. 2-propanol (5.0 volumes) was charged and the mixture was concentrated under reduced pressure and this process was repeated once. Tert-butyl methyl ether (5.0 volumes) was charged to the reactor. The mixture was agitated for about 2 hours, cooled to about 20° C. over about 2 hours, and agitated for about 1 h. The mixture was heated to about 60° C. and agitated for about 2 hours and then cooled to about 20° C. over about 2 hours. The slurry was then agitated for 10 hours at about 20° C. Tert-butyl methyl ether (3.0 volumes) was charged to the reactor and the slurry was filtered, rinsed with 20% 2-propanol in tert-butyl methyl ether (2.5 volumes), and dried at about 50° C. for about 8 hours to afford a dry solid that was then placed in a vial with about 80 mL of EtOH and ˜ 3 mL of water and seeds of potassium salt Form I. The sample was sonicated for ˜1 hour to yield a precipitate, and then stirred overnight at room temperature, filtered, and dried under nitrogen.

The potassium salt Form II XRPD pattern is shown FIG. 25 and is characterized by Tier 1 reflections at 5.6, 19.0, 25.6° 2θ, but also Tier 2 at 14.9, 18.1, 21.7° 2θ, and Tier 3 at 13.0, 16.2, 27.0° 2θ. A list of 2-theta peaks is provided below:

Peak Table

| Pos. | Rel. Int. |
|---|---|
| [°2Th.] | [%] |
| 5.6498 | 100.00 |
| 7.3593 | 15.01 |
| 13.0013 | 20.94 |
| 14.8561 | 34.99 |
| 16.2062 | 22.04 |
| 18.1349 | 36.72 |
| 18.9590 | 63.84 |
| 21.7218 | 36.85 |
| 25.5562 | 46.28 |
| 26.9590 | 30.14 |
| 29.0217 | 18.72 |
| 30.1946 | 14.27 |

The DSC curve is shown in FIG. 26 and displays an endothermic transition at about 18° C., a baseline shift at 227° C., an endothermic transition at about 260° C., and an exothermic transition at about 293° C. The TGA curve is shown in FIG. 27 and indicates that the phase is hydrated. The dynamic vapor sorption curve is shown in FIG. 28 and the data indicate that the form absorbs about ˜20% of water up to 95% RH at 25° C. The material was found to have converted to an amorphous form post experiment.

### Example 9. Compound of Formula I, Diethylamine Salt

Compound of Formula I, diethylamine salt is an unsolvated phase. It was isolated when about 30 mg of the compound of Formula I, Form I and about 10 mg of diethylamine were added to about 1 mL of MeCN, and the sample was slurried overnight to yield a precipitate that was filtered to dryness.

The diethylamine salt XRPD pattern is shown in FIG. 29 and is characterized by Tier 1 reflections at 10.7, 18.5, 19.3° 2θ, but also Tier 2 at 9.3, 20.4, 21.5° 2θ, and Tier 3 at 9.1, 11.8, 17.0° 2θ. A list of 2-theta peaks is provided below:

Peak Table

| Pos. | Rel. Int. |
|---|---|
| [°2Th.] | [%] |
| 9.0600 | 33.02 |
| 9.2858 | 59.35 |
| 10.6772 | 89.06 |
| 11.7972 | 43.10 |
| 13.5390 | 24.66 |
| 16.0415 | 28.61 |
| 17.0096 | 41.16 |
| 18.4981 | 68.99 |
| 19.3448 | 100.00 |
| 20.4488 | 66.46 |
| 21.4765 | 64.81 |
| 23.3941 | 24.79 |
| 25.8753 | 23.86 |
| 26.9302 | 13.33 |
| 29.9768 | 13.89 |
| 31.8091 | 13.95 |

The DSC curve is shown in FIG. 30 and displays an endothermic transition at about 115° C., an endothermic transition at about 128° C., an endothermic transition at about 151° C., and an endothermic transition at about 246° C. The TGA curve is shown in FIG. 31 and indicates that the phase is unsolvated. The dynamic vapor sorption curve is shown in FIG. 32 and the data indicate that the form absorbs about ˜0.14% of water up to 95% RH at 25° C. The material was found to not have changed form post experiment.

### Example 10. Compound of Formula I, Diethanolamine Salt

Compound of Formula I, diethanolamine salt is an unsolvated phase. It was isolated when ˜5000 mg of Compound of Formula I, form I was placed in a vial with ˜15 mL of DCM. ˜1.5 mL of diethanolamine was added along with ˜75 mL of heptane and the sample sonicated for 5 minutes until a precipitate formed. The mixture was then stirred for 3 days at room temperature and then suction filtered to dryness.

Diethanolamine salt XRPD pattern is shown in FIG. 33 and is characterized by Tier 1 reflections at 19.3, 20.4, 21.7° 2θ, but also Tier 2 at 18.2, 26.2, 30.4° 2θ, and Tier 3 at 6.7, 13.7, 23.5° 2θ. A list of 2-theta peaks is provided below:

Peak Table

| Pos. | Rel. Int. |
|---|---|
| [°2Th.] | [%] |
| 6.7260 | 35.01 |
| 9.6955 | 15.62 |
| 10.5501 | 29.19 |
| 13.7067 | 41.39 |
| 16.2291 | 28.31 |
| 17.5184 | 33.92 |
| 18.1604 | 54.48 |
| 19.2910 | 68.26 |
| 20.3546 | 93.10 |
| 21.6826 | 100.00 |
| 23.5064 | 39.27 |
| 24.4165 | 31.06 |
| 26.1929 | 41.71 |
| 28.0623 | 30.70 |
| 30.3657 | 42.58 |
| 31.9905 | 36.70 |
| 35.6863 | 23.91 |

The DSC curve is shown in FIG. 34 and displays an endothermic transition at about 176° C. and an endothermic transition at about 210° C. The TGA curve is shown in FIG. 35 and indicates that the phase is unsolvated. The dynamic vapor sorption curve is shown in FIG. 36 and the data indicate that the form absorbs about ˜17% of water up to 95% RH at 25° C. The material was found to not have changed form post experiment.

### Example 11. Compound of Formula I, Piperazine Co-Crystal

Compound of Formula I, piperazine co-crystal is a hydrated phase. It was isolated when about 30 mg of the compound of Formula I, Form I was placed in a vial with about 5 mL of acetone. About 6 mg of piperazine was added and the sample sonicated until a precipitate formed. The sample was then left as an open-faced vial to allow the sample to dry.

The piperazine co-crystal Form I XRPD pattern is shown in FIG. 37 and is characterized by Tier 1 reflections at 12.4, 16.2, 19.0° 2θ, but also Tier 2 at 6.0, 12.0, 15.0° 2θ, and Tier 3 at 6.7, 17.5, 21.7° 2θ. A list of 2-theta peaks is provided below:

Peak Table

| Pos. | Rel. Int. |
|---|---|
| [°2Th.] | [%] |
| 2.1207 | 16.89 |
| 4.2323 | 10.94 |
| 6.0067 | 45.59 |
| 6.7108 | 32.55 |
| 9.4674 | 9.43 |
| 11.9985 | 46.39 |
| 12.3900 | 48.01 |
| 14.9970 | 47.84 |
| 16.1860 | 100.00 |
| 17.5422 | 29.24 |
| 18.3121 | 20.41 |
| 19.0220 | 78.82 |
| 21.7037 | 37.07 |
| 22.9330 | 9.25 |
| 24.6718 | 4.99 |
| 27.0287 | 6.76 |
| 28.6697 | 6.53 |
| 29.8694 | 11.17 |

The DSC curve is shown in FIG. 38 and displays an endothermic transition at about 17° C., an endothermic transition at about 193° C., an exothermic transition at about 213° C., an endothermic transition at about 230° C., and an endothermic transition at about 243° C. The TGA curve is shown in FIG. 39 and indicates that the phase is hydrated. The dynamic vapor sorption curve is shown in FIG. 40 and the data indicate that the form absorbs about ˜3.5% of water up to 95% RH at 25° C. The material was found to not have changed form post experiment.

### Example 12. Compound of Formula I, Calcium Salt, Form I

### Example 12a

Compound of Formula I, calcium salt, Form I is a hydrated phase. It was isolated when around 1 equiv. of Compound of Formula I, Form I, around 6 volumes of ethanol, and around 2.5 volumes of water were charged to a reactor and stirred. Next around 1.05 equiv. of 50% w/v aq. potassium hydroxide was then charged and the mixture was agitated for about 24 hours at room temperature, then polish filtered into a clean reactor. Then around 0.2 volumes of heptane were added, followed by around 0.5 equiv. of a 1 M aq. calcium chloride solution charged over about 6 hours. The mixture was agitated for about 1 hour, then the slurry was then filtered, and the solids rinsed with around 5.0 volumes of water twice.

The Compound of Formula I, calcium salt, Form I XRPD pattern is shown in FIG. 41 and is characterized by reflections at 6.1, 5.6, 4.8, and 7.6° 2θ. A list of 2-theta peaks is provided below:

Peak Table

| Pos. | Rel. Int. |
|---|---|
| [°2Th.] | [%] |
| 4.759 | 32.23 |
| 5.5975 | 52.28 |
| 6.1208 | 100 |
| 7.6264 | 14.89 |

The DSC curve is shown in FIG. 42 and displays an endothermic transition at about 43 CC and an endothermic transition at about 291° C. The TGA curve is shown in FIG. 43 and indicates that the phase is hydrated. The dynamic vapor sorption curve is shown in FIG. 44 and the data indicate that the form absorbs about ˜18% of water up to 95% RH at 25° C.

### Example 12b

Compound of Formula I, calcium salt, Form I was also isolated when around 1 equiv. of Compound of Formula I, Form I, around 6 volumes of methanol, and around 3.0 volumes of water were charged to a reactor and stirred. Triethylamine (1.4 equiv.) was charged. The mixture was agitated for about 1 hour at room temperature, and polish filtered into a clean reactor. A IM aq. calcium chloride solution (0.5 equiv.) was charged over about 3 hours. The mixture was agitated for about 6 hours. The slurry was filtered, and the solids rinsed with a 50 vol % solution of methanol in water (3.0 volumes) twice, and dried to afford Compound of Formula I, calcium salt, Form I.

### Example 13. Compound of Formula I, Calcium Salt, Form II

Compound of Formula I, calcium salt, Form II is a hydrated phase. It was isolated when compound of Formula I, calcium salt, Form I was slurried around 4 day at room temperature in either 9:1 THF:water (v/v) or 8:2 THF:water (v/v).

The Compound of Formula I, calcium salt, Form II XRPD pattern is shown in FIG. 45 and is characterized by Tier 1 reflections at 6.4, 5.2, 5.6° 2θ, but also Tier 2 at 18.9, 21.9, 10.9° 2θ, and Tier 3 at 16.8, 15.1, 29.3° 2θ. A list of 2-theta peaks is provided below:

Peak Table

| Pos. | Rel. Int. |
|---|---|
| [°2Th.] | [%] |
| 5.2027 | 94.25 |
| 5.6215 | 83.25 |
| 6.4645 | 100 |
| 10.8889 | 25.26 |
| 15.0964 | 15.92 |
| 16.8257 | 23.38 |
| 18.9083 | 29.87 |
| 21.9068 | 27.32 |
| 24.9566 | 6.72 |
| 25.9705 | 6.49 |
| 27.8323 | 9.87 |
| 29.2844 | 15.82 |
| 31.4693 | 12.42 |

The DSC curve is shown in FIG. 46 and displays an endothermic transition at about 19° C., and an endothermic transition at about 320° C. The TGA curve is shown in FIG. 47 and indicates that the phase is hydrated.

### Example 14. Compound of Formula I, Calcium Salt, Form III

Compound of Formula I, calcium salt, Form III is a hydrated phase. It was isolated when compound of Formula I, calcium salt, Form I was slurred around 4 day at room temperature in either 9:1 IPA:water (v/v), 8:2 IPA:water (v/v), or 7:3 IPA:water (v/v). Alternatively, it was isolated when Form I was slurred around 14 day at room temperature in either acetone, 6:4 MeOH:water (v/v), 5:5 MeOH:water (v/v), 3:7 EtOH:water (v/v), 2:8 EtOH:water (v/v), or 1:9 IPA:water (v/v).

The compound of Formula I, calcium salt, Form III XRPD pattern is shown in FIG. 48 and is characterized by Tier 1 reflections at 4.2, 5.2, 6.2° 2θ, but also Tier 2 at 13.9, 21.0, 15.7° 2θ, and Tier 3 at 12.6, 7.5, 10.6° 2θ.

Peak Table

| Pos. | Rel. Int. |
|---|---|
| [°2Th.] | [%] |
| 4.2027 | 100 |
| 5.2494 | 64.58 |
| 6.1747 | 62.82 |
| 7.4813 | 15.12 |
| 10.6013 | 12.69 |
| 12.5542 | 17.46 |
| 13.9203 | 27.71 |
| 15.6797 | 19.8 |
| 21.0148 | 25.12 |

The DSC curve is shown in FIG. 49 and displays an endothermic transition at about 17° C., an endothermic transition at about 93° C., and an endothermic transition at about 286° C. The TGA curve is shown in FIG. 50 and indicates that the phase is hydrated. The dynamic vapor sorption curve is shown in FIG. 51 and the data indicate that the form absorbs about ˜14% of water up to 95% RH at 25° C.

### Example 15. Compound of Formula I, Calcium Salt, Form IV

Compound of Formula I, calcium salt, Form IV is a hydrated phase. It was isolated when around 1 equiv. of compound of Formula I Form I, around 6 volumes of ethanol, and around 2.5 volumes of water were charged to a reactor and stirred. Next around 1.05 equiv. of 50% w/v aq. potassium hydroxide was then charged and the mixture was agitated for about 24 hours at room temperature, then polish filtered into a clean reactor. Then around 0.2 volumes of Heptane were added, followed by around 0.5 equiv. of a 1 M aq. calcium chloride solution charged over about 6 hours. The mixture was agitated for about 1 hour, then the slurry was then filtered, and the mother liquors retained and held at room temperature for around 2 weeks to yield a solid that was filtered and dried.

The compound of Formula I, calcium salt, Form IV XRPD pattern is shown in FIG. 52 and is characterized by Tier 1 reflections at 6.5, 5.2, 20.7° 2θ, but also Tier 2 at 7.0, 18.9, 13.2° 2θ, and Tier 3 at 17.8, 9.6, 22.9° 2θ.

Peak Table

| Pos. | |
|---|---|
| [°2Th.] | Rel. Int. |
| 5.1906 | 97.5 |
| 6.5315 | 100 |
| 6.9516 | 63.68 |
| 8.4207 | 11.94 |
| 9.5864 | 38.78 |
| 12.1583 | 7.54 |
| 13.1534 | 44.74 |
| 14.7177 | 14.96 |
| 15.6964 | 20.69 |
| 17.8258 | 43.97 |
| 18.895 | 56.04 |
| 20.7192 | 65.87 |
| 22.8522 | 33.95 |
| 24.3624 | 29.15 |
| 26.1189 | 11.88 |
| 28.138 | 24.29 |

The DSC curve is shown in FIG. 53 and displays an endothermic transition at about 17° C., and a baseline shift around 315° C. The TGA curve is shown in FIG. 54 and indicates that the phase is hydrated. The dynamic vapor sorption curve is shown in FIG. 55 and the data indicate that the form absorbs about ˜3% of water up to 95% RH at 25° C.

### Example 16. Compound of Formula I, Calcium Salt, Form V

Compound of Formula I, calcium salt, Form V is a hydrated phase. It was isolated when to a reactor was charged around 1 equiv. of compound of Formula I, Form I, around 6 volumes of methanol, and around 3 volumes of ammonium buffer (3 M NH₄OH, 1 M NH₄Cl). The mixture was agitated at about 25° C. for about 10 minutes then a 1 M solution of CaCl₂ (0.5 equiv) was added over about 3 h. The resulting mixture was aged for about 18 h then filtered, and the cake washed twice with 3 volumes of water. The wet-cake was dried under vacuum at about 50° C.

The compound of Formula I, calcium salt, Form V XRPD pattern is shown in FIG. 56 and is characterized by Tier 1 reflections at 6.3, 9.2, 4.7° 2θ, but also Tier 2 at 17.5, 6.6, 7.3° 2θ, and Tier 3 at 19.8, 10.3, 21.9° 2θ.

Peak Table

| Pos. | Rel. Int. |
|---|---|
| [°2Th.] | [%] |
| 4.667286 | 85.49 |
| 6.325596 | 100 |
| 6.574366 | 58.89 |
| 7.337722 | 41.98 |
| 9.219071 | 92.48 |
| 10.33942 | 28.4 |
| 11.76972 | 25.41 |
| 13.8499 | 17.35 |
| 17.51082 | 71.54 |
| 19.82631 | 37.1 |
| 21.93564 | 26.21 |
| 23.9084 | 19.19 |
| 28.46511 | 11.11 |

The DSC curve is shown in FIG. 57 and displays an endothermic transition at about 34° C., and an endothermic transition at about 1962° C. The TGA curve is shown in FIG. 58 and indicates that the phase is hydrated. The dynamic vapor sorption curve is shown in FIG. 59 and the data indicate that the form absorbs about ˜0.13% of water up to 95% RH at 25° C.

### Example 17. Compound of Formula I, Calcium Salt, Form VI

Compound of Formula I, calcium salt, Form VI is a hydrated phase. It was isolated when compound of Formula I, calcium salt, Form V was slurried at room temperature in MeOH for about 14 days.

The compound of Formula I, calcium salt, Form VI XRPD pattern is shown in FIG. 60 and is characterized by Tier 1 reflections at 6.2, 5.0, 5.8° 2θ, but also Tier 2 at 19.1, 18.2, 21.0° 2θ, and Tier 3 at 21.7, 10.0, 10.3° 2θ.

Peak Table

| Pos. | Rel. Int. |
|---|---|
| [°2Th.] | [%] |
| 4.97 | 63.7 |
| 5.83 | 53.94 |
| 6.21 | 100 |
| 7.90 | 10.79 |
| 8.65 | 6.8 |
| 9.95 | 22.63 |
| 10.34 | 21.71 |
| 12.61 | 15.78 |
| 13.22 | 19.38 |
| 14.50 | 20.38 |
| 15.77 | 12.66 |
| 18.22 | 29.81 |
| 19.11 | 35.21 |
| 20.98 | 27.28 |
| 21.71 | 26.79 |
| 22.61 | 16.35 |
| 24.99 | 20.77 |
| 26.59 | 8.56 |
| 29.60 | 9.96 |

The DSC curve is shown in FIG. 61 and displays an endothermic transition at about 51° C., and an endothermic transition at about 262° C. The TGA curve is shown in FIG. 62 and indicates that the phase is hydrated. The dynamic vapor sorption curve is shown in FIG. 63 and the data indicate that the form absorbs about ˜0.1% of water up to 95% RH at 25° C.

### Example 18. Compound of Formula I, Calcium Salt, Amorphous

### Example 18a

Compound of Formula I, calcium salt, amorphous was isolated when approximately 2 grams potassium salt was placed in a vial with about 20 mL of 1:1 EtOH:water (v/v) to yield a solution. Then around 500 mg of calcium chloride was added to yield a precipitate and the sample then stirred at RT for 3 nights then solids isolated. Alternatively, it was made when around 1.00 equiv. of Compound of Formula I, Form I and around 5.0 volumes of ethanol were charged to a reactor and stirred at room temperature. Next around 1.2 equiv. of 50% w/v aq. potassium hydroxide was charged, followed by around 3.5 volumes of water. The mixture was agitated for about 2 hours at room temperature, then polish filtered into a clean reactor. Then around 0.5 equiv. of 1 M aq. calcium chloride solution was charged over about 19 hours while agitating. The slurry was then filtered, the solids rinsed with around 10.0 volumes of water three times, and then dried. Its XRPD pattern is shown in FIG. 64 and is characterized by broad bumps. The DSC curve is shown in FIG. 65 indicating a baseline shift around 288° C.

### Example 18b

The compound of Formula I, calcium salt, amorphous was also made when to a reactor was charged the compound of Formula I, Form I (1.00 equiv), calcium hydroxide (0.5 equiv), methanol (4 volumes), tetrahydrofuran (4.0 volumes), and triethylamine (2.0 equiv). The mixture was agitated at about 25° C. then water (1.0 volume) was charged, and the resulting mixture aged for about 12 hours. The reaction stream was then polish filtered into a clean reactor and water (9.0 volumes) was charged over about 2 hours, and the resulting slurry aged for about 12 hours. The slurry was then filtered, and the cake washed twice with 3 volumes of 20:80 methanol/water containing 1 vol % triethylamine. The wet-cake was dried under vacuum at 60° C.

### Example 18c

The compound of Formula I, calcium salt, amorphous was also made when to a reactor was charged the compound of Formula I, Form I (1.00 equiv, scaling factor), calcium oxide (0.5 equiv), and methanol (5 volumes). The mixture was heated to about 65° C. and aged for about 12 hours. To the mixture was charged tetrahydrofuran (5 volumes), triethylamine (2.0 equiv), and water (1.0 volume) and the resulting mixture aged for about 1 hour. The reaction stream was then polish filtered into a clean reactor and water (9.0 volumes) was charged over about 2 hours, and the resulting slurry aged for about 12 hours. The slurry was then filtered, and the cake washed twice with 3 volumes of 20:80 methanol/water containing 1 vol % triethylamine. The wet-cake was dried under vacuum at 60° C.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

1. A crystalline form of a compound of Formula: 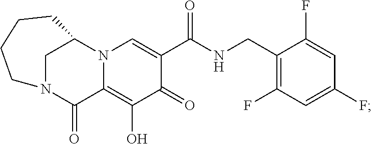 [SMILES: O=C(NCc1c(F)cc(F)cc1F)c1cn2c(c(O)c1=O)C(=O)N1CCCC[C@H]2C1]
wherein the crystalline form is selected from Form I, Form II, Form III, and Form IV.

2. The crystalline form of claim 1, wherein the crystalline form is Form I and is characterized by an X-ray powder diffraction (XRPD) pattern comprising °2-θ peaks (+0.2° 2-θ) at 17.8°, 22.0°, and 25.4°.

3.-12. (canceled)

13. The crystalline form of claim 1, wherein the crystalline form is Form II and is characterized by an XRPD pattern comprising 2-θ peaks (±0.2° 2-θ) at 9.5°, 10.9°, and 21.2°.

14.-19. (canceled)

20. The crystalline form of claim 1, wherein the crystalline form is Form III and is characterized by an XRPD pattern comprising 2-θ peaks (+0.2° 2-θ) at 15.8°, 17.3°, and 26.4°.

21.-32. (canceled)

33. The crystalline form of claim 1, wherein the crystalline form is Form IV and is characterized by an XRPD pattern comprising 2-θ peaks (+0.2° 2-θ) at 4.1°, 12.3°, and 16.4°.

34.-42. (canceled)

43. A compound of Formula: 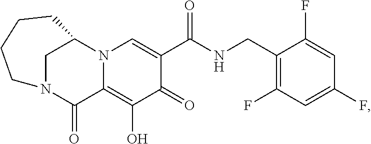 [SMILES: O=C(NCc1c(F)cc(F)cc1F)c1cn2c(c(O)c1=O)C(=O)N1CCCC[C@H]2C1]
wherein the compound is amorphous.

44. The compound of claim 43, characterized by a XRPD pattern substantially as set forth in FIG. 14.

46. A sodium salt of a compound of Formula: 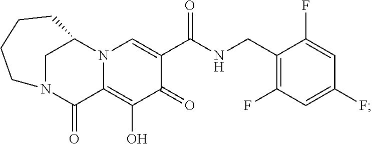 [SMILES: O=C(NCc1c(F)cc(F)cc1F)c1cn2c(c(O)c1=O)C(=O)N1CCCC[C@H]2C1]
wherein the salt is selected from a sodium salt, a potassium salt, a calcium salt, a diethylamine salt, and a diethanolamine salt.

47. A crystalline form of the sodium salt of claim 46, wherein the crystalline form is characterized by an XRPD pattern comprising 2-θ peaks (±0.2° 2-θ) at 6.5°, 20.2°, and 23.5°.

48.-57. (canceled)

58. A crystalline form of the potassium salt of claim 46, wherein the crystalline form is Form I characterized by an XRPD pattern comprising 2-θ peaks (±0.2° 2-θ) at 6.6°, 19.7°, and 23.9°.

59.-67. (canceled)

68. A crystalline form of the potassium salt of claim 46, wherein the crystalline form is Form II characterized by an XRPD pattern comprising 2-θ peaks (±0.2° 2-θ) at 5.6°, 19.0°, and 25.6°.

69.-78. (canceled)

79. The calcium salt of claim 46, wherein the salt is Form I characterized by an XRPD pattern comprising any three °2-θ peaks (±0.2° 2-θ) selected from 5.6°, 4.8°, 6.1°, and 7.6°.

80.-88. (canceled)

89. A crystalline form of the calcium salt of claim 46, wherein the crystalline form is Form II characterized by an XRPD pattern comprising ° 2-θ peaks (±0.2° 2-θ) at 5.2°, 5.6°, and 6.4°.

90.-98. (canceled)

99. A crystalline form of the calcium salt of claim 46, wherein the crystalline form is Form III characterized by an XRPD pattern comprising ° 2-θ peaks (±0.2° 2-θ) at 4.2°, 5.2°, and 6.2°.

100.-107. (canceled)

108. A crystalline form of the calcium salt of claim 46, wherein the crystalline form is Form IV characterized by an XRPD pattern comprising ° 2-θ peaks (±0.2° 2-θ) at 5.2°, 6.5°, and 20.7°.

109.-116. (canceled)

117. A crystalline form of the calcium salt of claim 46, wherein the crystalline form is Form V characterized by an XRPD pattern comprising ° 2-θ peaks (±0.2° 2-θ) at 4.7°, 6.3°, and 9.2°.

118.-125. (canceled)

126. A crystalline form of the calcium salt of claim 46, wherein the crystalline form is Form VI characterized by an XRPD pattern comprising ° 2-θ peaks (=0.2° 2-θ) at 5.0°, 5.8°, and 6.2°.

127.-134. (canceled)

135. The calcium salt of claim 46, wherein the calcium salt is amorphous.

136.-138. (canceled)

139. A crystalline form of the diethylamine salt of claim 46, wherein the crystalline form is characterized by an XRPD pattern comprising 2-θ peaks (±0.2° 2-θ) at 10.7°, 18.5°, and 19.3°.

140.-151. (canceled)

152. A crystalline form of the diethanolamine salt of claim 46, wherein the crystalline form is characterized by an XRPD pattern comprising 2-θ peaks (=0.2° 2-θ) at 19.3°, 20.4°, 21.7°.

153.-162. (canceled)

163. A co-crystal comprising a compound of Formula: 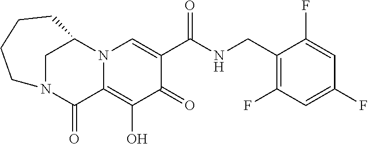 [SMILES: O=C(NCc1c(F)cc(F)cc1F)c1cn2c(c(O)c1=O)C(=O)N1CCCC[C@H]2C1]
and piperazine.

164. A co-crystal of claim 163, wherein the co-crystal is characterized by an XRPD pattern comprising 2-θ peaks (±0.2° 2-θ) at 12.4°, 16.2°, and 19.0°.

165.-173. (canceled)

174. A pharmaceutical composition comprising:
(i) a therapeutically effective amount of the crystalline form of claim 1; and
(ii) a pharmaceutically acceptable carrier or excipient.

175.-185. (canceled)

186. A method of treating an HIV infection in a human having or at risk of having the infection, comprising administering to the human a therapeutically effective amount of:
the crystalline form of claim 1.

187.-210. (canceled)

211. A pharmaceutical composition comprising:
(i) a therapeutically effective amount of the salt of claim 46; and
(ii) a pharmaceutically acceptable carrier or excipient.

212. A method of treating an HIV infection in a human having or at risk of having the infection, comprising administering to the human a therapeutically effective amount of the salt of claim 46.

213. A pharmaceutical composition comprising:
(i) a therapeutically effective amount of the co-crystal of claim 163; and
(ii) a pharmaceutically acceptable carrier or excipient.

214. A method of treating an HIV infection in a human having or at risk of having the infection, comprising administering to the human a therapeutically effective amount of the co-crystal of claim 163.
